(19) United States
(12) Patent Application Publication
(10) Pub. No.: US 2009/0263400 A1
(43) Pub. Date: Oct. 22, 2009

(54) OSTEOPOROSIS ASSOCIATED MARKERS AND METHODS OF USE THEREOF

(75) Inventors: Mickey Urdea, Alamo, CA (US); Michael McKenna, Oakland, CA (US); Patrick Arensdorf, Palo Alto, CA (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: TETHYS BIOSCIENCE, INC., Emeryville, CA (US)

(21) Appl. No.: 12/408,104

(22) Filed: Mar. 20, 2009

Related U.S. Application Data
(63) Continuation of application No. 11/703,400, filed on Feb. 6, 2007, now abandoned.
(60) Provisional application No. 60/771,077, filed on Feb. 6, 2006.

(51) Int. Cl. A61K 39/395; G01N 33/68; C12Q 1/68

(52) U.S. Cl. 424/141.1; 436/86; 435/6

(57) ABSTRACT

Disclosed are methods of identifying subjects with osteoporosis or osteopenia, subjects at risk for developing osteoporosis, osteopenia, and bone fractures, methods of evaluating the effectiveness of osteoporosis treatments in subjects with osteoporosis or osteopenia, and methods of selecting therapies for treating osteoporosis or osteopenia, using biomarkers.

### INCORPORATION BY REFERENCE

This application is a continuation of U.S. patent application Ser. No. 11/703,400 filed Feb. 6, 2007, which claims priority from U.S. Provisional Application Ser. No. 60/771,077, filed on Feb. 6, 2006.

Each of the applications and patents cited in this text, as well as each document or reference cited in each of the applications and patents (including during the prosecution of each issued patent; “application cited documents”), and each of the U.S. and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference. Documents incorporated by reference into this text may be employed in the practice of the invention.

### FIELD OF THE INVENTION

The present invention relates generally to the identification of biological markers associated with an increased risk of developing bone fractures, osteoporosis and pre-osteoporosis.

### BACKGROUND OF THE INVENTION

Osteoporosis is a systemic skeletal disorder characterized by low bone mass, microarchitectural deterioration of bone tissue, and compromised bone strength resulting in an increased risk of bone fractures. Osteoporosis can be further characterized as either primary or secondary. Primary osteoporosis can occur in both genders at all ages, but often follows menopause in women and occurs later in life in men. In contrast, secondary osteoporosis is a result of medications, other conditions, risk factors, or diseases. Examples include, but are not limited to, glucocorticoid-induced osteoporosis, hypogonadism, cancers, other endocrine disorders, celiac disease, genetic disorders, inflammatory diseases, malnutritive and/or malabsorption syndromes.

Throughout life, bone is continuously remodeled with resorption of old bone (catabolic process) performed by osteoclasts and deposition of new bone (anabolic process) performed by osteoblasts. Bone remodeling is not a random process and takes place in focal bone multicellular units (BMUs), which are remodeling units comprising osteoblasts, osteoclasts, and their precursors, in which resorption and formation are coupled. Bone resorption is likely the initial event that occurs in response to local mechanical stress signals. The reduction in bone density found in osteoporosis results from an imbalance between resorption and formation, wherein the rate of resorption exceeds that of formation. Osteoporosis represents a continuum, in which multiple pathogenetic mechanisms converge to cause loss of bone mass and microarchitectural deterioration of skeletal structure. Osteoporosis is likely to be caused by complex interactions among local and systemic regulators of bone cell function. The heterogeneity of osteoporosis may be due not only to differences in the production of systemic and local regulators, but also to changes in receptors, signal transduction mechanisms, nuclear transcription factors, and enzymes that produce or inactivate local regulators.

Bone strength reflects the integration of two main features: bone density and bone quality. Bone density is expressed as grams of mineral per area or volume and, in any given individual, is determined by peak bone mass attained and subsequent amount of bone loss. Bone quality refers to architecture, turnover, damage accumulation (i.e., microfractures) and mineralization. A fracture frequently occurs when trauma is applied to osteoporotic bone, which is of a lower bone density. Thus, osteoporosis is a significant risk factor for bone fractures.

The incidence of bone fractures is high in individuals with osteoporosis and increases with age. Osteoporotic fractures, particularly vertebral fractures, can be associated with chronic disabling pain. The impact of osteoporosis on other body systems, such as gastrointestinal, respiratory, genitourinary, and craniofacial, has also been reported. Each year, an estimated 1.5 million individuals suffer a fracture due to bone disease. Roughly 4 in 10 Caucasian women aged 50 or older in the United States will experience a hip, spine, or wrist fracture sometime during the remainder of their lives. It is predicted that the lifetime risk of bone fractures will increase for all ethnic groups as life expectancy increases.

Osteoporosis is typically detected by a bone mineral density test, however, at the time of an initial bone fracture, the majority of affected individuals are not aware that they have low bone density or are at risk for osteoporosis, nor that they have various other risk factors for fracture that indicate a state of pre-osteoporosis. These include osteopenia (which represents example of pre-osteoporosis characterized by intermediate lowered bone density, between normal and that found in osteoporosis), but also other pre-osteoporosis such as conditions of decreased sex hormone production, vitamin deficiency, and hyperparathyroidism, among others. Bone mineral density tests are helpful in determining how much bone mineral is present and has already been lost, however these tests often produce inconsistent results among the population, and even among different bones of the same individual. Further, bone density tests cannot measure the rate of bone loss and consequently, fail to measure the rate of progression to or of osteoporosis. In the United States, it is estimated that 34 million individuals have osteopenia, and over 10 million have osteoporosis, with both together representing approximately 55 percent of the population 50 years of age and older.

Additionally, several individual biomarkers of bone metabolism have also been recently proposed as new measures of bone health, such as NTX, CTX, PYD, DPD, BSP, TRACP, Bone ALP, OC, and PICP or PINP, among others. While these biomarkers may be more sensitive than earlier generation markers, such as total Alkaline Phosphatase (ALP) and Hydroxyproline (Hyp or OHP), in detecting abnormalities in bone turnover rate, several limitations remain of such individual biomarkers. Despite that most of these markers may be classified as markers of bone formation or as markers of bone resorption, many markers reflect both processes, albeit to varying degrees. Most of these markers are also present in tissues other than bone and may therefore be influenced by nonskeletal processes as well. Changes in such markers are usually not disease specific, but reflect alterations in skeletal metabolism independent of their cause. Finally, significant pre-analytical and analytical variability exists to such biomarkers, due to factors that may be either uncontrollable (such as age, gender, ethnicity, menopausal status, hormone or medication use, disease or recent fractures, and the nature of the biomarkers themselves), requiring adjustment of biomarker results or interpretation, or controllable (by sampling method, sample type, circadian cycle, menstrual cycle, diet, exercise effects, etc.) As a result, their clinical use in the management of the individual patient has not been clearly defined and is a matter of debate (see Delmas et al., The Use of Biochemical Markers of Bone Turnover in Osteoporosis. Osteoporosis International (2000) Suppl 6: S2-S17 and also Seibel, Biochemical Markers of Bone Turnover, Clin Biochem Rev (2005) 26: 97-122, which are hereby incorporated by reference in their entirety).

There remains an unmet need in the art for predictive and prognostic assays to determine whether individuals are indeed at risk for bone fractures, or of developing osteoporosis and/or osteopenia. Such assays would have significant utility used either alone or in conjunction with a bone mineral density test. Development of such assays would permit earlier intervention to reduce the likelihood of bone fracture and delay the onset of osteoporosis in affected individuals.

### SUMMARY OF THE INVENTION

The present invention relates in part to the discovery that certain biological markers, such as proteins, nucleic acids, polymorphisms, metabolites, and other analytes are present in subjects with an increased risk of bone metabolic disorders, such as osteoporosis, osteopenia and/or other pre-osteoporosis condition, which may result in an increased risk of bone fractures. Accordingly, the invention provides biological markers of bone metabolism that can be used to monitor or assess the risk of subjects developing osteoporosis and/or osteopenia, to diagnose or identify subjects with osteoporosis and/or osteopenia, to monitor the risk of bone fracture, to monitor subjects that are undergoing therapies for bone fractures, osteoporosis, osteopenia, and/or pre-osteoporosis, and to select therapies for use in treating subjects with bone fractures, osteoporosis, pre-osteoporosis and/or osteopenia, or for use in subjects who are at risk for developing bone fractures, osteoporosis, pre-osteoporosis, osteopenia, or other disorders in bone metabolism, including those which may result in an increased risk of bone fracture. The biomarkers are collectively referred to herein as “OSTEORISKMARKERS”, the proteins are collectively referred to herein as “OSTEORISKMARKER polypeptides” or “OSTEORISKMARKER proteins”. The corresponding encoded nucleic acids are referred to as “OSTEORISKMARKER nucleic acids” or “OSTEORISKMARKER polynucleotides”. The corresponding metabolites are referred to as “OSTEORISKMARKER metabolites”. Non-analyte physiological markers of health status (e.g., age, gender, bone density, bone mass, and other non-analyte measurements commonly used as conventional risk factors) are referred to as “OSTEORISKMARKER physiology”. Calculated indices created from mathematically combining measurements of one or more of the aforementioned classes of OSTEORISKMARKERS are referred to as “OSTEORISKMARKER indices”. “OSTEORISKMARKER” or “OSTEORISKMARKERS” refers to one or more OSTEORISKMARKER proteins, OSTEORISKMARKER analytes, OSTEORISKMARKER nucleic acids, OSTEORISKMARKER metabolites, OSTEORISKMARKER physiology, and/or OSTEORISKMARKER indices.

A subject having a bone metabolic disorder such as osteoporosis, pre-osteoporosis, and/or osteopenia can be identified by measuring the levels of an effective amount (which can be one or more) of OSTEORISKMARKERS in a subject-derived sample and the levels are then compared to a reference value. Alterations in the level of biomarkers, such as proteins, polypeptides, nucleic acids and polynucleotides, polymorphisms of proteins, polypeptides, nucleic acids, and polynucleotides, mutated proteins, polypeptides, nucleic acids, and polynucleotides, or alterations in the molecular quantities of metabolites or other analytes (such as elemental calcium), or of other physiology in the subject sample compared to the reference value are then identified. A reference value can be relative to a number or value derived from population studies, including without limitation, such subjects having similar body or bone mass index (BMI) or similar bone mineral densities, subjects of the same or similar age range, subjects in the same or similar ethnic group, or, in female subjects, pre-menopausal or post-menopausal subjects, or relative to the starting sample of a subject undergoing treatment for a bone health disorder, such as osteoporosis, pre-osteoporosis, or osteopenia.

In one embodiment of the present invention, the reference value is the level of OSTEORISKMARKERS in a control sample derived from one or more subjects who do not have osteoporosis, pre-osteoporosis, or osteopenia. Such subjects who do not have osteoporosis, pre-osteoporosis, or osteopenia can be verified as those subjects who have a T-score above −1 on a bone mineral density test or can be verified by another diagnostic test of bone metabolism known in the art, such as but not limited to, bone biopsy.

A subject predisposed to developing a bone metabolic disorder such as osteoporosis, pre-osteoporosis, and/or osteopenia, or at increased risk of developing osteoporosis, pre-osteoporosis, osteopenia, or bone fractures, can be identified by measuring the levels of an effective amount (which can be one or more) of OSTEORISKMARKERS in a subject-derived sample and the levels are then compared to a reference value. Alterations in the level of expression or amounts of proteins, polypeptides, nucleic acids and polynucleotides, polymorphisms of proteins, polypeptides, nucleic acids, and polynucleotides, or alterations in the molecular quantities of metabolites or other analytes, or of other physiology, in the subject sample compared to the reference value are then identified. A reference value can be relative to a number or value derived from population studies including without limitation, such subjects having similar body or bone mass index (BMI) or similar bone mineral densities, subjects of the same or similar age range, subjects in the same or similar ethnic group, or, in female subjects, pre-menopausal or post-menopausal subjects, or relative to a value obtained from a starting sample of a subject undergoing treatment for a bone health disorder, or subjects who are not at risk or at low risk for developing osteoporosis, pre-osteoporosis, or osteopenia.

In one embodiment of the present invention, the reference value is the level of OSTEORISKMARKERS in a control sample derived from one or more subjects who are not at risk or at low risk for developing osteoporosis, pre-osteoporosis, or osteopenia. Such subjects who are not at risk or at low risk for developing osteoporosis, pre-osteoporosis, or osteopenia can be verified by comparing the bone densities of the subjects against a number derived from longitudinal studies of subjects from which the likelihood of osteoporotic, pre-osteoporotic, or osteopenic progression can be determined, including without limitation, such subjects having similar body or bone mass index (BMI) or similar bone mineral densities, subjects of the same or similar age range, subjects in the same or similar ethnic group, or, in female subjects, pre-menopausal or post-menopausal subjects.

In another embodiment, the reference value is an index value or a baseline value. An index value or baseline value is a composite sample of an effective amount of OSTEORISKMARKERS from one or more subjects who do not have a bone health disorder, such as osteoporosis, pre-osteoporosis, or osteopenia. In this embodiment, to make comparisons to the subject-derived sample, the level of OSTEORISKMARKERS are similarly calculated and compared to the index value. Optionally, subjects identified as having osteoporosis, pre-osteoporosis, or osteopenia, or being at increased risk of developing osteoporosis, pre-osteoporosis, or osteopenia are chosen to receive a therapeutic regimen to reverse, halt or slow the progression of osteoporosis or osteopenia, or decrease or prevent the risk of developing osteoporosis, pre-osteoporosis, or osteopenia.

The progression of osteoporosis, pre-osteoporosis, or osteopenia, or effectiveness of a bone fracture, osteoporosis or osteopenia treatment regimen can be monitored by detecting an OSTEORISKMARKER in an effective amount (which can be one or more) of samples obtained from a subject over time and comparing the amount of OSTEORISKMARKERS detected. For example, a first sample can be obtained prior to the subject receiving treatment and one or more subsequent samples are optionally taken after or during treatment of the subject. Osteoporosis, pre-osteoporosis, and osteopenia are defined to be progressive (or, alternatively, the treatment does not prevent progression) if the amount of OSTEORISKMARKER changes over time relative to the reference value, whereas osteoporosis and osteopenia are not progressive if the levels of OSTEORISKMARKERS remains constant over time (relative to the reference population, or “constant” as used herein). The term “constant” as used in the context of the present invention is construed to include changes over time, including those changes to subsequent OSTEORISKMARKER amounts that are closer with respect to the reference value than those in the first sample.

Additionally, therapeutic or prophylactic agents suitable for administration to a particular subject can be identified by detecting an OSTEORISKMARKER in an effective amount (which can be one or more) in a sample obtained from a subject, exposing the subject-derived sample to a test compound that determines the level of an effective amount (which can be one or more) of OSTEORISKMARKERS in the subject-derived sample. Accordingly, treatments or therapeutic regimens for use in subjects having osteoporosis, pre-osteoporosis, or osteopenia, or subjects at risk for developing osteoporosis, pre-osteoporosis, osteopenia, or bone fractures can be selected based on the levels of OSTEORISKMARKERS in samples obtained from the subjects and compared to a reference value. Two or more treatments or therapeutic regimens can be evaluated in parallel to determine which treatment or therapeutic regimen would be the most efficacious for use in a subject to prevent, reverse, or delay onset, or slow progression of osteoporosis, osteopenia, or bone fracture.

The present invention further provides a method for screening for changes in marker levels associated with osteoporosis, by determining the level of an effective amount (which can be one or more) of OSTEORISKMARKERS in a subject-derived sample, comparing the level of the OSTEORISKMARKERS in a reference sample, and identifying alterations in levels in the subject sample compared to the reference sample.

A “subject” as defined herein includes a mammal, such as but not limited to, a human, a non-human primate, a mouse, a rat, a dog, a cat, a horse, or a cow. The subject can be male or female. A subject can include those who have not been previously diagnosed as having osteoporosis, pre-osteoporosis, or osteopenia, or who have not previously had bone fractures. Alternatively, a subject can also include those who have already been diagnosed as having osteoporosis, pre-osteoporosis, osteopenia or bone fractures. Optionally, the subject has been previously treated with therapeutic agents, or with other therapies and treatment regimens for osteoporosis, pre-osteoporosis, and osteopenia, such as, but not limited to, dietary supplements (such as calcium or vitamin supplements), bisphosphonates (for example, alendronate and the like), selective estrogen receptor modulators (SERMs), hormonal agents, calcitonin, anabolic drugs, or combinations thereof. Treatment regimens can also encompass exercise regimens. A subject can also include those who are suffering from, or at risk of developing osteoporosis, pre-osteoporosis, osteopenia or bone fractures, such as those who exhibit known risk factors for osteoporosis, pre-osteoporosis, or osteopenia, or who do not score normally (for example, scores at or below −1) on a bone mineral density test, i.e., those who have decreased bone mineral density. For example, a subject diagnosed with osteoporosis according to World Health Organization (WHO) definitions has T-scores at or below −2.5 on a bone mineral density test. A subject diagnosed with osteopenia according to WHO definitions has T-scores between −1 and −2.5 on a bone mineral density test (See Woolf & Pfleger, Burden of Major Musculoskeletal Conditions, Bulletin of the World Health Organization (2003) 81: 646-656).

A “sample” in the context of the present invention is a biological sample isolated from a subject and can include, for example, serum, blood plasma, blood cells, ascites fluid, interstitital fluid (such as gingival crevicular fluid), bone marrow, sputum, cerebrospinal fluid, saliva, or urine.

One or more, preferably two or more OSTEORISKMARKERS can be detected in the practice of the present invention. For example, one (1), two (2), five (5), ten (10), twenty (20), forty (40), fifty (50), seventy-five (75), one hundred (100) or more OSTEORISKMARKERS can be detected. In some aspects, all 191 OSTEORISKMARKERS disclosed herein can be detected. Preferred ranges from which the number of OSTEORISKMARKERS can be detected include ranges bounded by any minimum selected from between one and 191, particularly one, two, five, ten, twenty, fifty, seventy-five, one hundred, one hundred and twenty five, paired with any maximum up to the total known OSTEORISKMARKERS, particularly five, ten, twenty, fifty, and seventy-five. Particularly preferred ranges include one to two (1-2), two to five (2-5), two to ten (2-10), two to fifty (2-50), two to seventy-five (2-75), two to one hundred (2-100), five to ten (5-10), five to twenty (5-20), five to fifty (5-50), five to seventy-five (5-75), five to one hundred (5-100), ten to twenty (10-20), ten to fifty (10-50), ten to seventy-five (10-75), ten to one hundred (10-100), twenty to fifty (20-50), twenty to seventy-five (20-75), twenty to one hundred (20-100), fifty to seventy-five (50-75), fifty to one hundred (50-100), one hundred to one hundred and twenty-five (100->125), one hundred and twenty-five to one hundred and fifty (125->150), one hundred and fifty to one hundred and seventy five (150->175), and one hundred and seventy five to more than one hundred and ninety (175->190⁺).

Optionally, other markers known to be associated with bone health disorders such as osteoporosis, osteopenia, pre-osteoporosis and bone fractures can be detected. The OSTEORISKMARKERS can be detected by any means known in the art. For example, OSTEORISKMARKERS can be detected electrophoretically or immunochemically, by RNA quantification, or generically by any technique involving an attractive force, covalent cross-linking, or binding event between the OSTEORISKMARKER of interest and detection and/or capture materials (which may be an antibody, an antibody fragment, or any biological or synthetic polymer, including, without limitation, proteins, nucleic acids (as in aptamers), and plastic polymeric substrates such as those formed by molecular imprinting techniques). Immunochemical detection includes, for example, radio-immunoassay, immunoblotting, immunofluorescence, or enzyme-linked immunosorbent assay (ELISA), but are not limited to these detection methods. One skilled in the art is versed in various immunochemical detection methods, such as those described in “Current Protocols in Molecular Biology” (Ausubel, F. M. et al. John Wiley & Sons, 1987). For example, an OSTEORISKMARKER protein can be detected using an anti-OSTEORISKMARKER protein antibody, and the amount of antigen-antibody complex can be detected as a measure of the OSTEORISKMARKER protein in the sample. Post-translational modifications of OSTEORISKMARKER proteins can also be detected, as well as changes in the enzymatic activity of certain OSTEORISKMARKER proteins. Alternatively, OSTEORISKMARKER nucleic acids, such as RNA or DNA, can be detected. For example, an OSTEORISKMARKER nucleic acid can be identified by detecting hybridization, i.e., on a silicon chip, or an OSTEORISKMARKER RNA or DNA probe to a transcript in the test sample and measured by i.e., Northern or Southern analysis. An OSTEORISKMARKER nucleic acid, such as RNA, can also be identified by RNA quantification, such as, without limitation, polymerase chain reaction (PCR), quantitative reverse-transcription polymerase chain reaction (RT-PCR), target amplification methods (TMA), bDNA methods such as signal amplification methods, and the like.

Optionally, OSTEORISKMARKER metabolites and other analytes can be detected. Metabolites and other analytes can be detected in numerous ways known to the skilled artisan, including, without limitation, refractive index spectroscopy (RI), ultraviolet spectroscopy (UV), fluorescence analysis, radiochemical analysis, near-infrared spectroscopy (near IR), nuclear magnetic resonance spectroscopy (NMR), light scattering analysis (LS), mass spectrometry (including matrix-assisted laser desorption ionization-time of flight, or MALDI-TOF), pyrolysis mass spectrometry, nephelometry, dispersive Raman spectroscopy, gas chromatography optionally combined with mass spectrometry, liquid chromatography optionally combined with mass spectrometry, ion spray spectroscopy combined with mass spectrometry, capillary electrophoresis, NMR, and IR detection. Other OSTEORISKMARKER may be detected directly by virtue of their chemical or electrochemical reactivity, e.g. by means of clinical or analytical chemistry.

Alterations in OSTEORISKMARKER levels, including OSTEORISKMARKER indices and other pattern recognition of multiple OSTEORISKMARKERS, are preferably statistically significant. By “statistically significant”, it is meant that the alteration is greater than what might be expected to happen by chance alone. Statistical significance can be determined by methods known in the art. An alteration is statistically significant if the p-value is at least 0.05. Preferably, the p-value is 0.04, 0.04, 0.02. 0.01, 0.005, 0.001 or less.

The invention also concerns osteoporosis or pre-osteoporosis reference molecular profiles, which can comprise a pattern of marker levels of an effective amount of one or more of the OSTEORISKMARKERS of the invention, taken from one or more subjects who do not have osteoporosis or pre-osteoporosis. The present invention also provides osteoporosis or pre-osteoporosis subject molecular profiles, which can comprise a pattern of marker levels of an effective amount of one or more OSTEORISKMARKERS of the invention, taken from one or more subjects who have osteoporosis or pre-osteoporosis, are at risk for developing osteoporosis or pre-osteoporosis, or are being treated for osteoporosis or pre-osteoporosis.

The present invention also comprises a kit with a detection reagent that binds to one or more OSTEORISKMARKER proteins, nucleic acids, polymorphisms, metabolites, or other analytes. Also provided by the invention is an array of detection reagents, i.e., antibodies and/or oligonucleotides that can bind to one or more OSTEORISKMARKER proteins or nucleic acids, respectively. In one embodiment, the OSTEORISKMARKER are proteins and the array contains antibodies that bind an effective amount of OSTEORISKMARKERS 1-191 sufficient to measure a statistically significant alteration in OSTEORISKMARKER levels compared to a reference value. In another embodiment, the OSTEORISKMARKERS are nucleic acids and the array contains oligonucleotides or aptamers that bind an effective amount of OSTEORISKMARKERS 1-191 sufficient to measure a statistically significant alteration in OSTEORISKMARKER levels compared to a reference value.

Also provided by the present invention is a method for treating one or more subjects at risk for developing osteoporosis, pre-osteoporosis, osteopenia or bone fracture, comprising: detecting the presence of increased levels of one or more different OSTEORISKMARKERS present in a sample from the one or more subjects; and treating the one or more subjects with one or more bone mineral content-modulating drugs until altered levels of the one or more different OSTEORISKMARKERS return to a baseline value measured in one or more subjects at low risk for developing osteoporosis, pre-osteoporosis, osteopenia, or bone fracture.

The bone mineral content-modulating drug can comprise biphosphonates, (such as alendronate, risedronate, etidronate, pamidronate, ibandronate, clodronate), selective estrogen receptor modulators (i.e. SERMs; such as raloxifene, tamoxifen, toremifine), strontium ranelate, low dose and/or recombinant peptide fragments of parathyroid hormone (such as teriparatide), estrogen/progesterone replacement therapies, monoclonal antibodies, inhibitors of receptor activator of nuclear factor κB ligand (RANKL) (such as denosumab and osteoprotegerin), inhibitors of cathepsin K, antagonists of integrin Avβ3, calcitonin, calcium supplements and vitamin D supplements.

Also provided by the present invention is a method for treating one or more subjects having osteoporosis, pre-osteoporosis, or osteopenia comprising: detecting the presence of increased levels of one or more different OSTEORISKMARKERS present in a sample from the one or more subjects; and treating the one or more subjects with one or more bone mineral content-modulating drugs until altered levels of the one or more different OSTEORISKMARKERS return to a baseline value measured in one or more subjects at low risk for developing osteoporosis, pre-osteoporosis, or osteopenia.

The present invention also concerns OSTEORISKMARKER panels that can comprise one or more OSTEORISKMAKERS indicative of a physiological or biochemical pathway as described herein, and as set forth in FIG. 4. The physiological or biochemical pathway can be selected from the group consisting of osteoclast metabolism, bone mineralization and/or calcification, skeletal development, muscle cell metabolism, eicosanoid metabolism, other metabolism, or other bone-related physiology. The OSTEORISKMARKER panels of the invention can also comprise combinations of OSTEORISKMARKERS of the various physiological or biochemical pathways of FIG. 4, wherein the panel can be selected from the group consisting of Categories 1-10 as set forth in FIG. 5.

Alternatively, or additionally, the present invention also provides OSTEORISKMARKER panels that comprise one or more OSTEORISKMARKERS indicative of bone resorption, bone formation, or both bone resorption and bone formation associated with osteoporosis or pre-osteoporosis. The OSTEORISKMARKER panels of the present invention can comprise OSTEORISKMARKERS indicative of bone formation and bone resorption as set forth in FIG. 3.

The present invention also provides OSTEORISKMARKER panels that comprise OSTEORISKMARKERS that are categorized into “clusters.” A representative number of clusters is set forth in FIG. 6. Accordingly, one embodiment of the OSTEORISKMARKER panels of the invention contain clusters selected from the group consisting of Cluster 1 through 11.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from the following detailed description and claims.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIG. 1A-1AA are graphic illustrations of the molecular pathways listed within the Kyoto University Encyclopedia of Genes and Genomes (KEGG) which feature three or more OSTEORISKMARKERS, identified by their common HUGO gene name abbreviation or alias, in each disclosed canonical pathway.

FIGS. 1A-1, 1A-2, and 1A-3 depict OSTEORISKMARKERS involved in cytokine-cytokine receptor interactions as shown in KEGG pathway hsa04060.

FIGS. 1B-1, 1B-2, and 1B-3 depict OSTEORISKMARKERS involved in neuroactive ligand-receptor interactions as shown in KEGG pathway hsa04080.

FIGS. 1C-1, 1C-2 and 1C-3 depict OSTEORISKMARKERS involved in mitogen-activated protein kinase (MAPK) interactions as shown in KEGG pathway hsa04010.

FIGS. 1D-1 and 1D-2 depict OSTEORISKMARKERS involved in Janus kinase-signal transducers and activators of transcription (JAK-STAT) interactions as shown in KEGG pathway hsa04630.

FIGS. 1E-1, 1E-2, and 1E-3 depict OSTEORISKMARKERS involved in Wnt signaling interactions as shown in KEGG pathway hsa04310.

FIGS. 1F-1, 1F-2, and 1F-3 depict OSTEORISKMARKERS involved in focal adhesions as shown in KEGG pathway hsa04510.

FIGS. 1G-1, 1G-2, and 1G-3 show OSTEORISKMARKERS involved in hematopoietic cell lineage interactions as depicted in KEGG pathway hsa04640.

FIGS. 1H-1, 1H-2, and 1H-3 show OSTEORISKMARKERS involved in TGF-β signaling interactions as depicted in KEGG pathway hsa04350.

FIGS. 1I-1 and 1I-2 show OSTEORISKMARKERS involved in extracellular matrix (ECM) receptor interactions as depicted in KEGG pathway hsa04512.

FIGS. 1J-1 and 1J-2 show OSTEORISKMARKERS involved in adipocytokine signaling interactions as depicted in KEGG pathway hsa04920.

FIG. 1K shows OSTEORISKMARKERS involved in Type I Diabetes Mellitus as depicted in KEGG pathway hsa04940.

FIG. 1L shows OSTEORISKMARKERS involved in cell junction interactions as depicted in KEGG pathway hsa01430.

FIGS. 1M-1 and 1M-2 depict OSTEORISKMARKERS involved in antigen processing and presentation as shown in KEGG pathway hsa04612.

FIGS. 1N-1, 1N-2, and 1N-3 depict OSTEORISKMARKERS involved in Toll-like Receptor signaling as shown in KEGG pathway hsa04620.

FIGS. 10-1 and 10-2 depict OSTEORISKMARKERS involved in T-cell Receptor signaling as shown in KEGG pathway hsa04660.

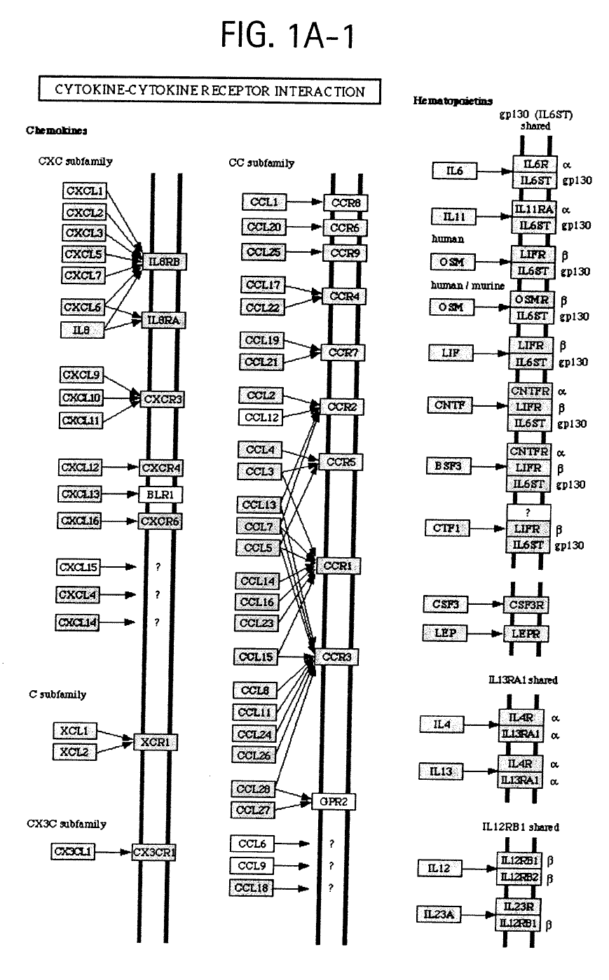

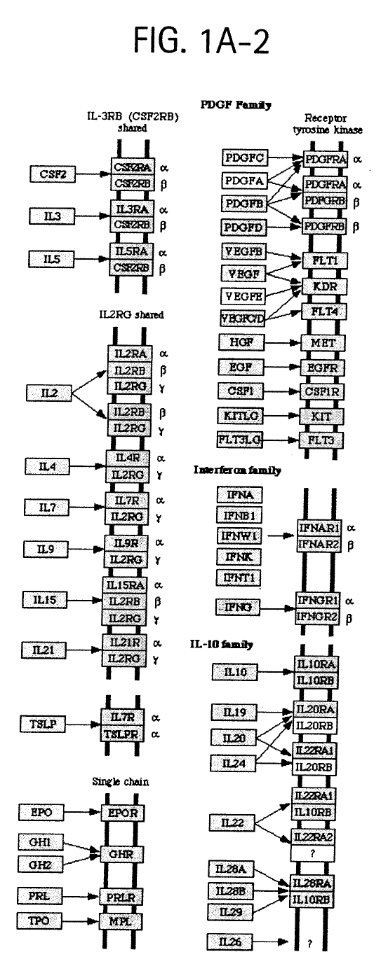

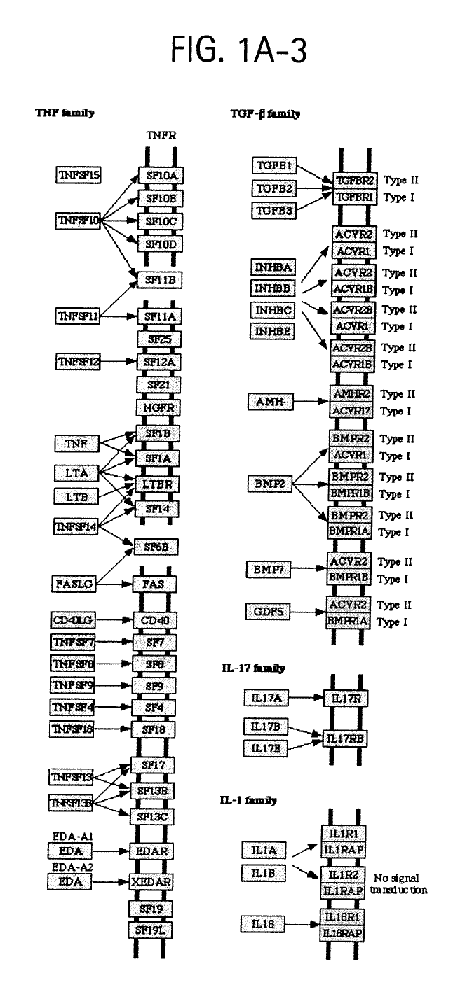

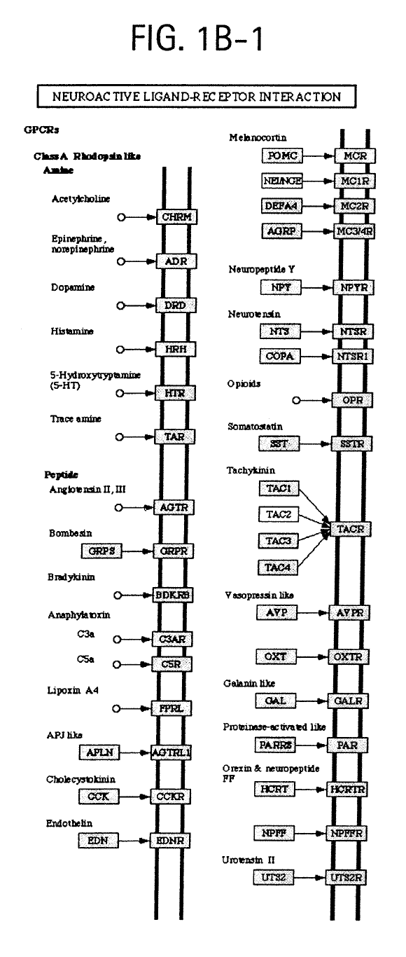

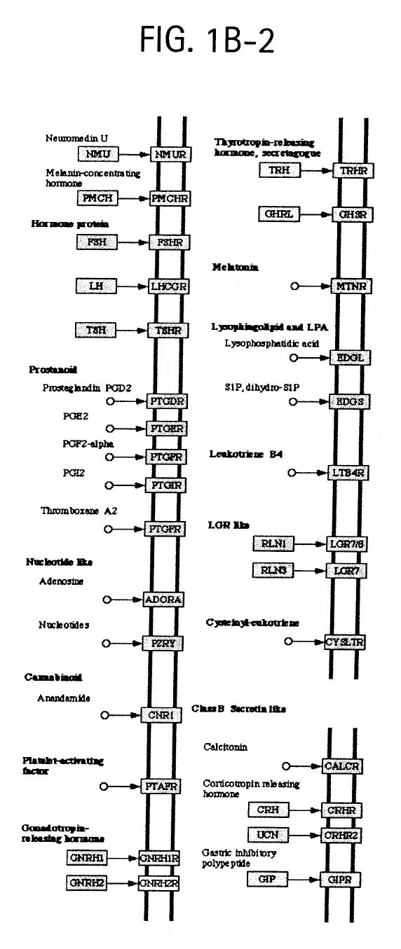

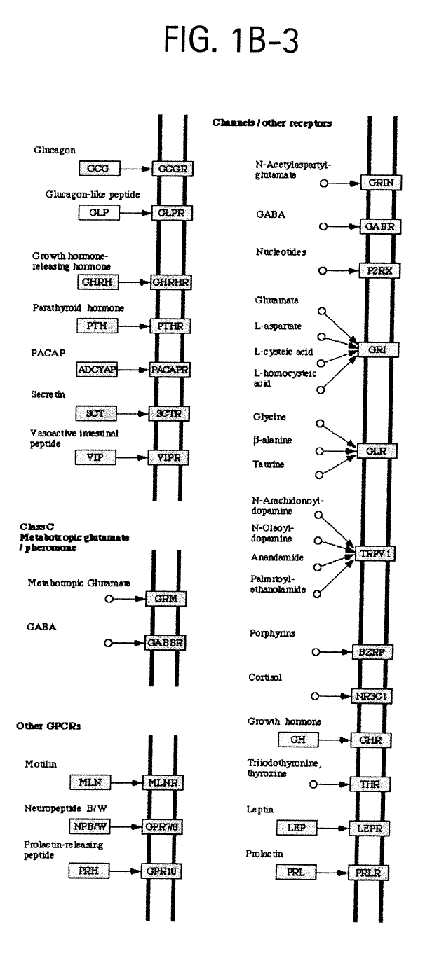

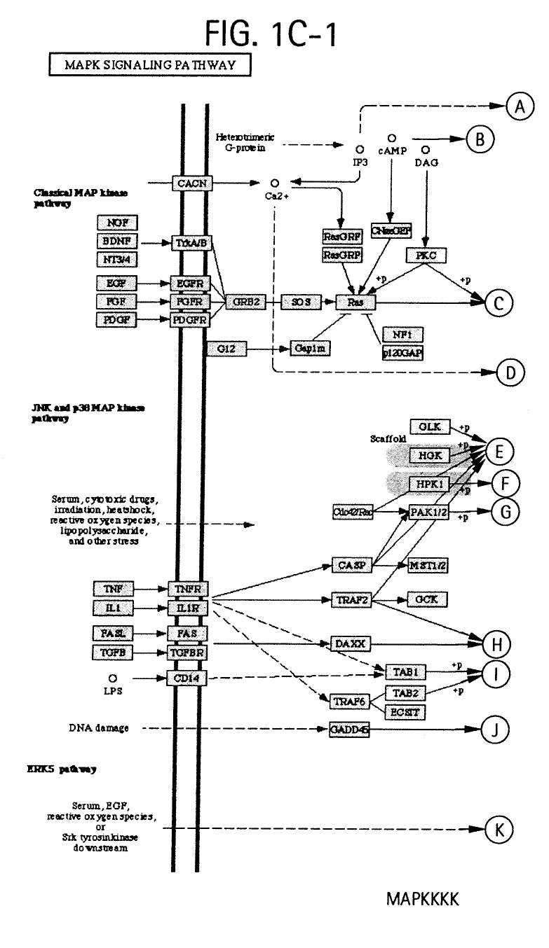

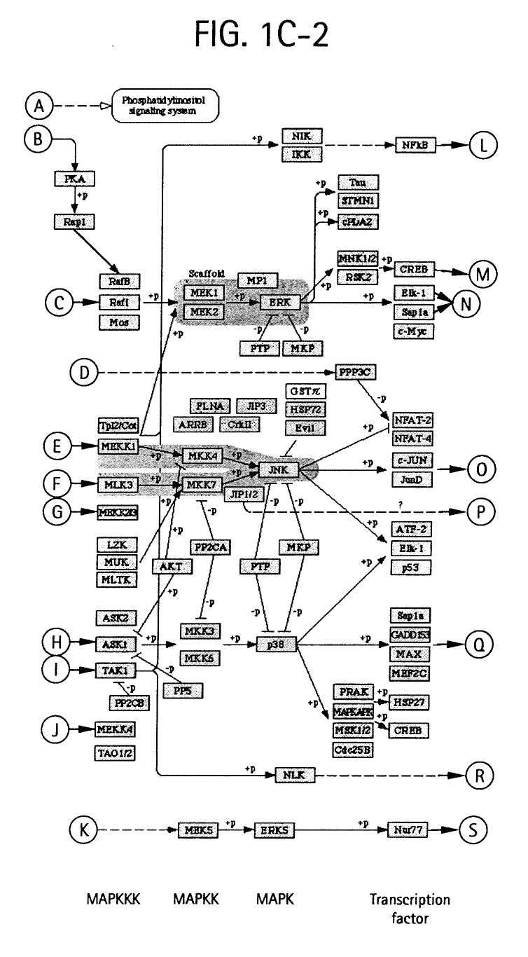

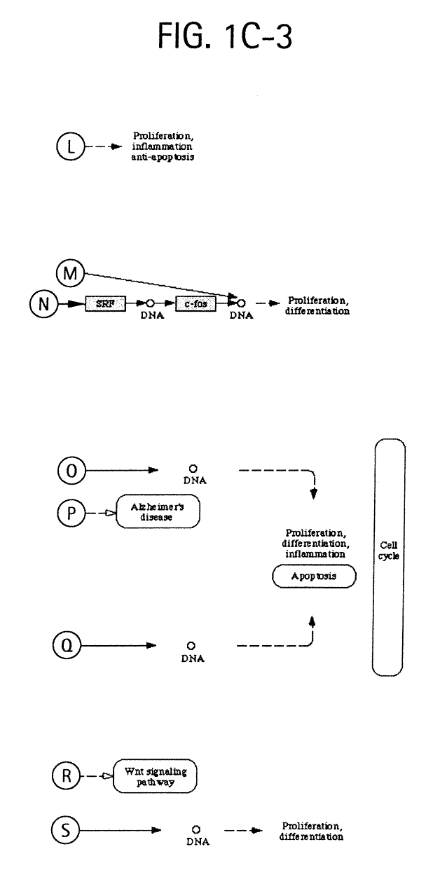

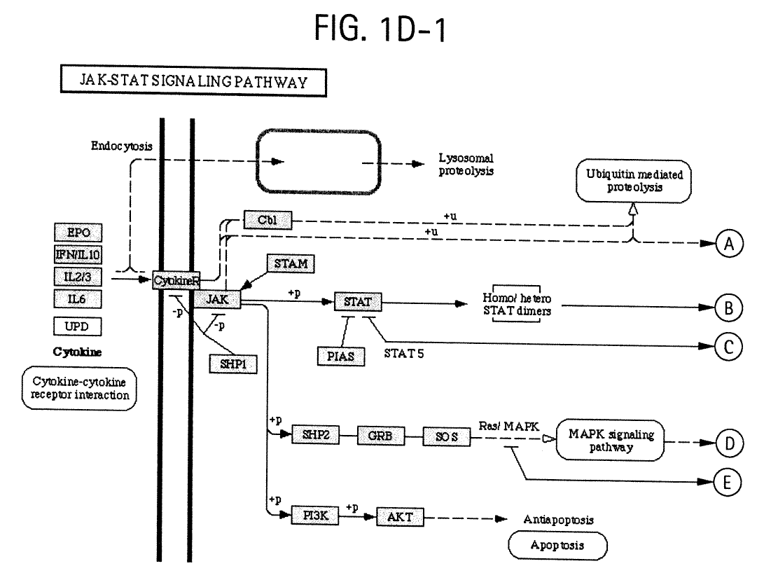

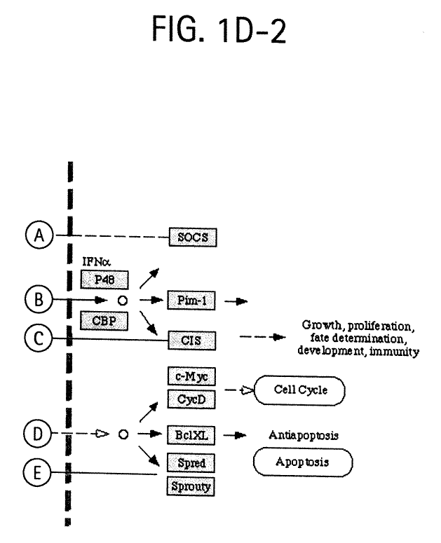

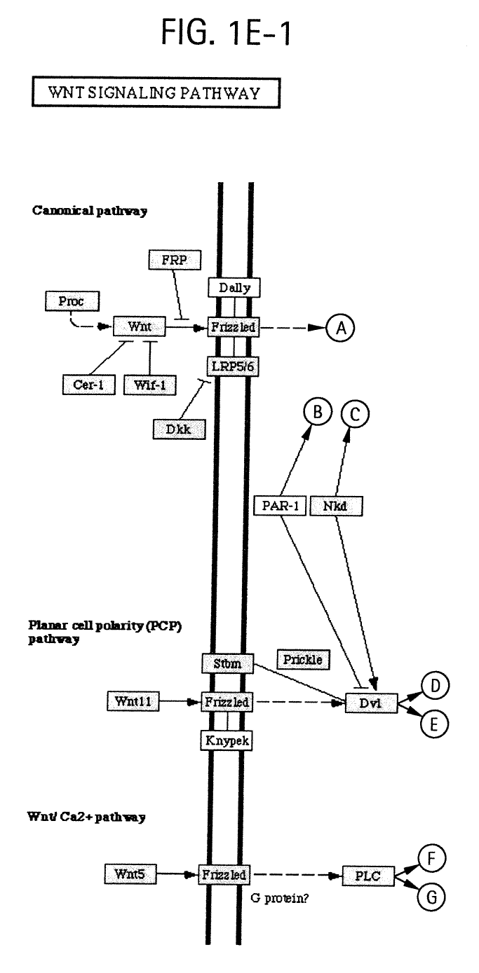

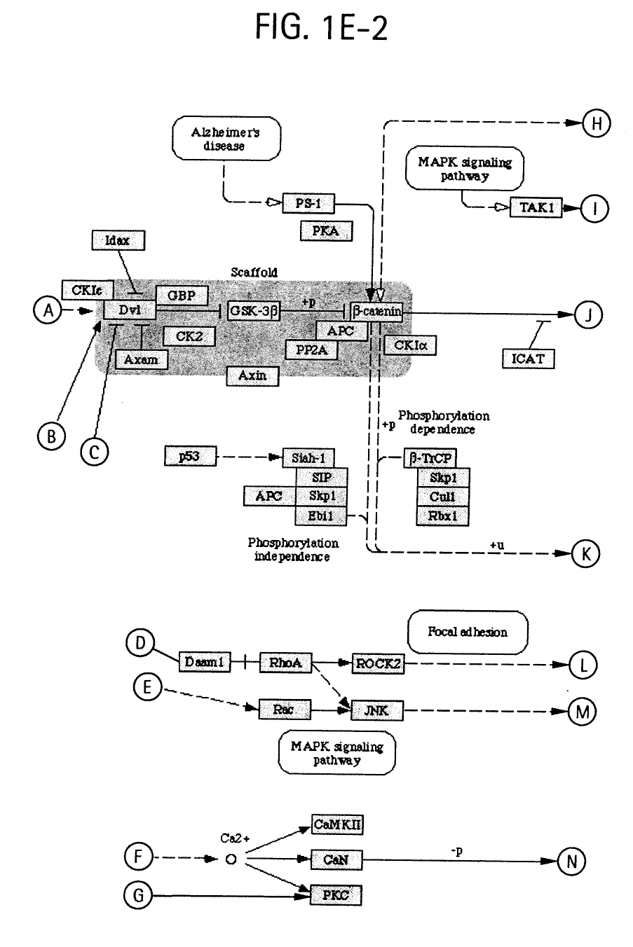

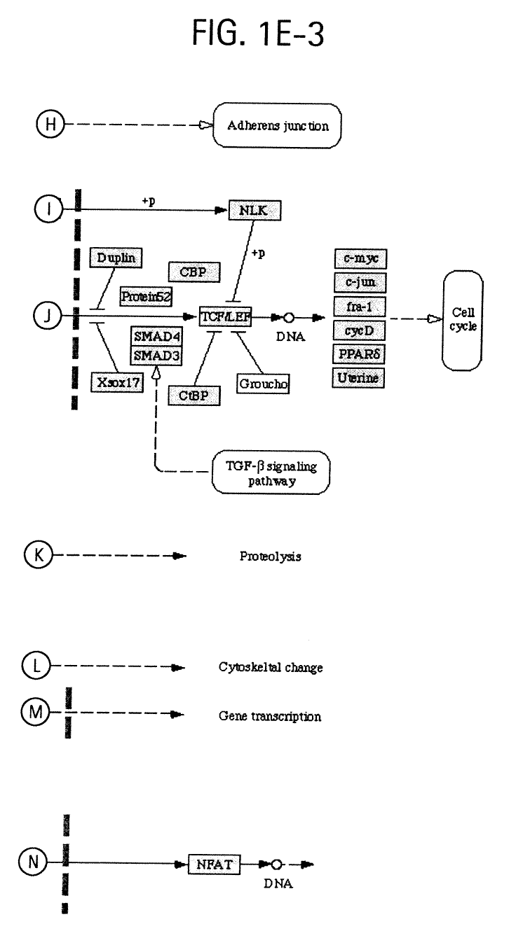

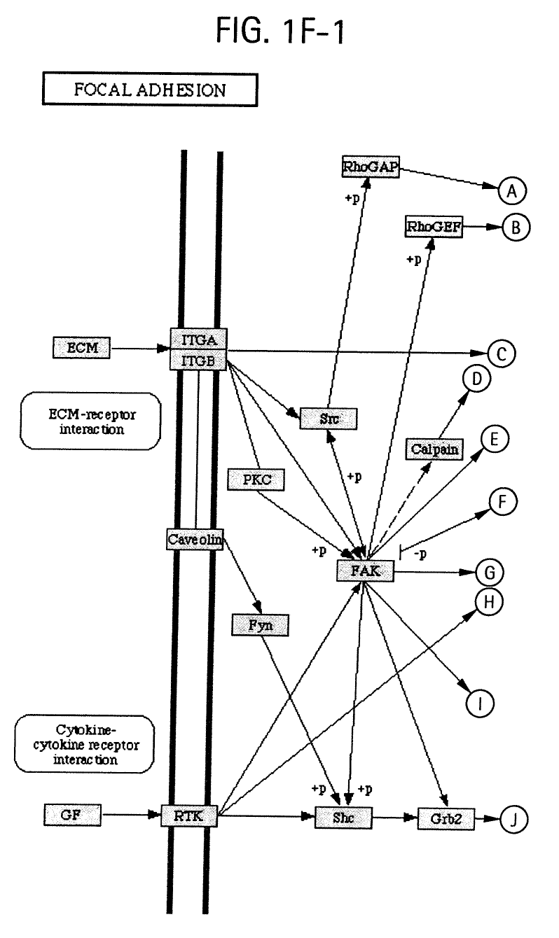

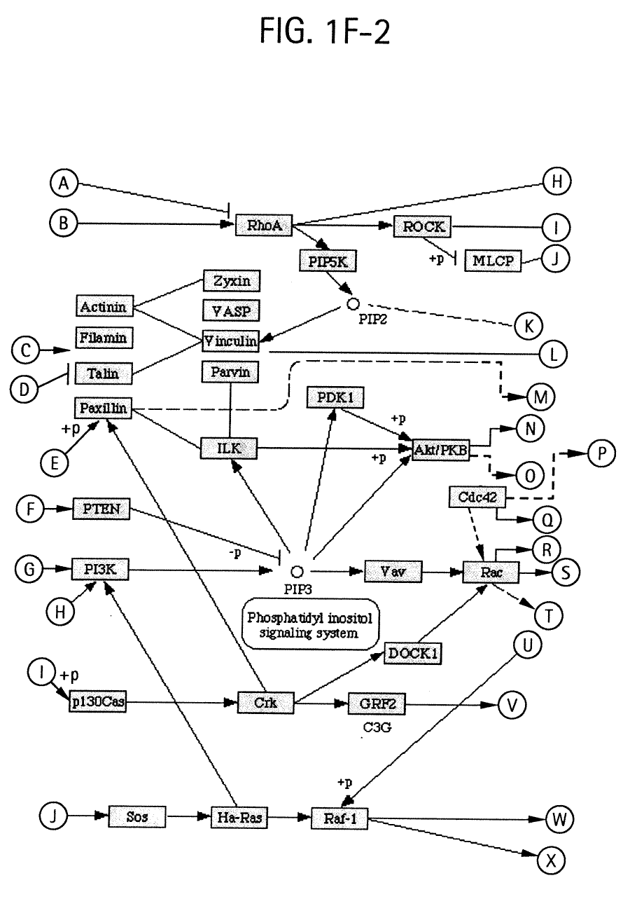

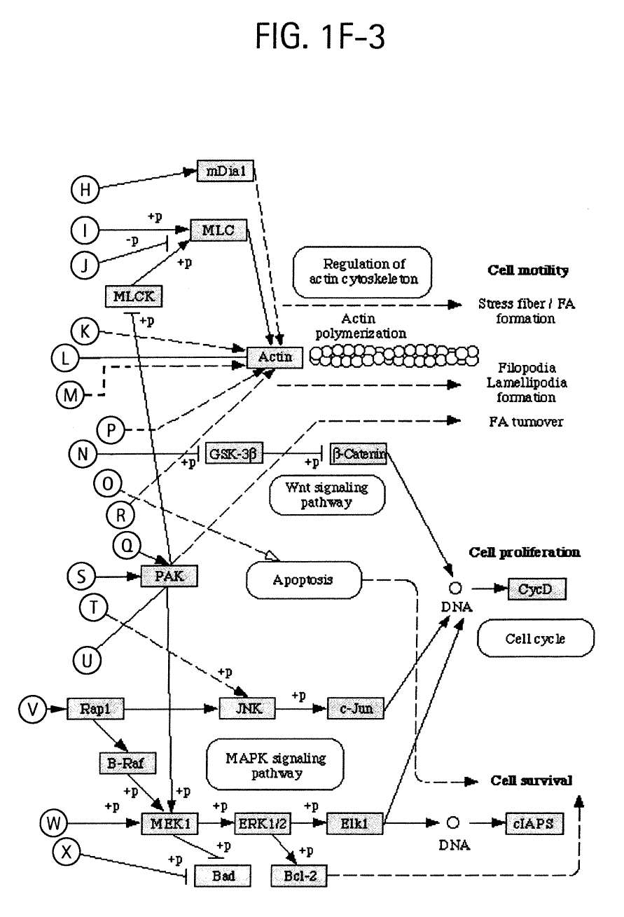

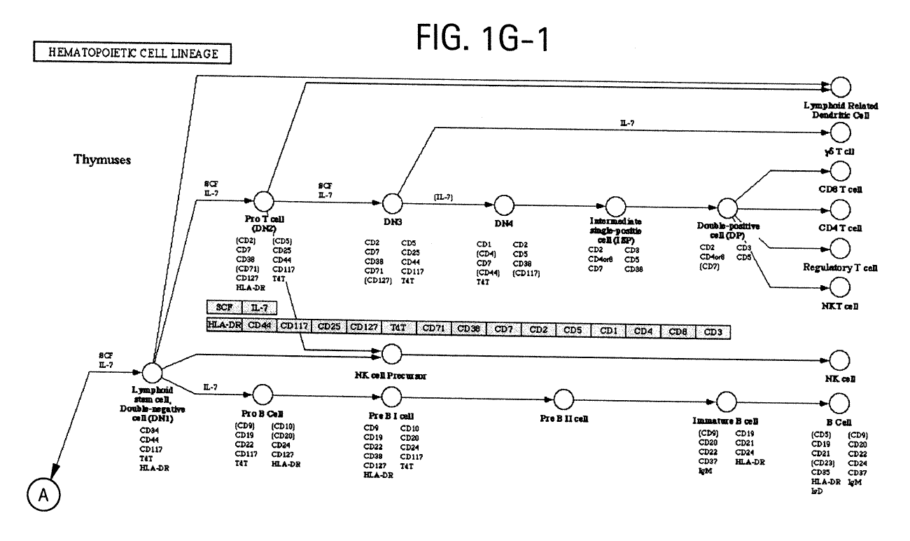

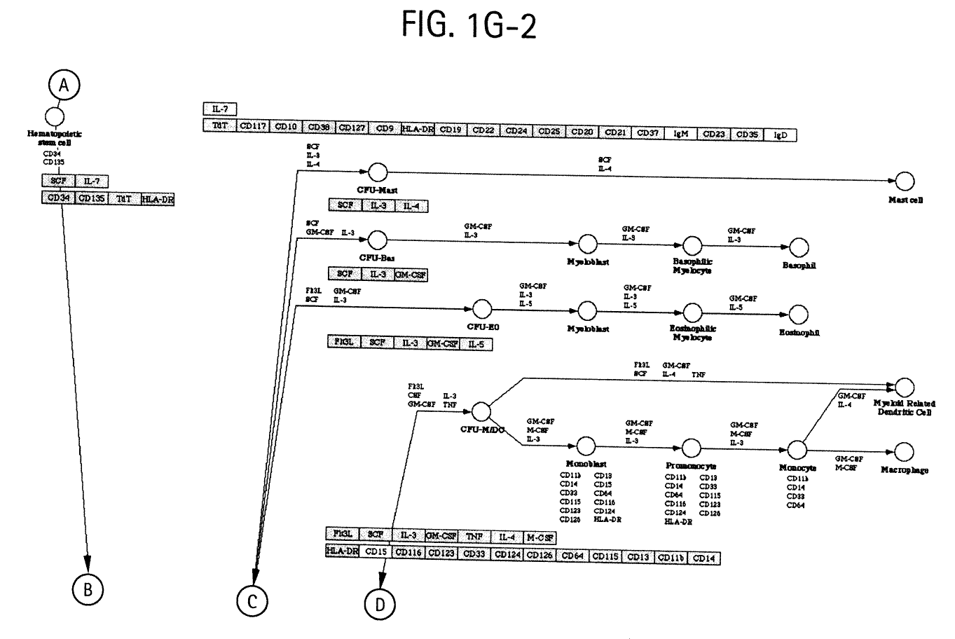

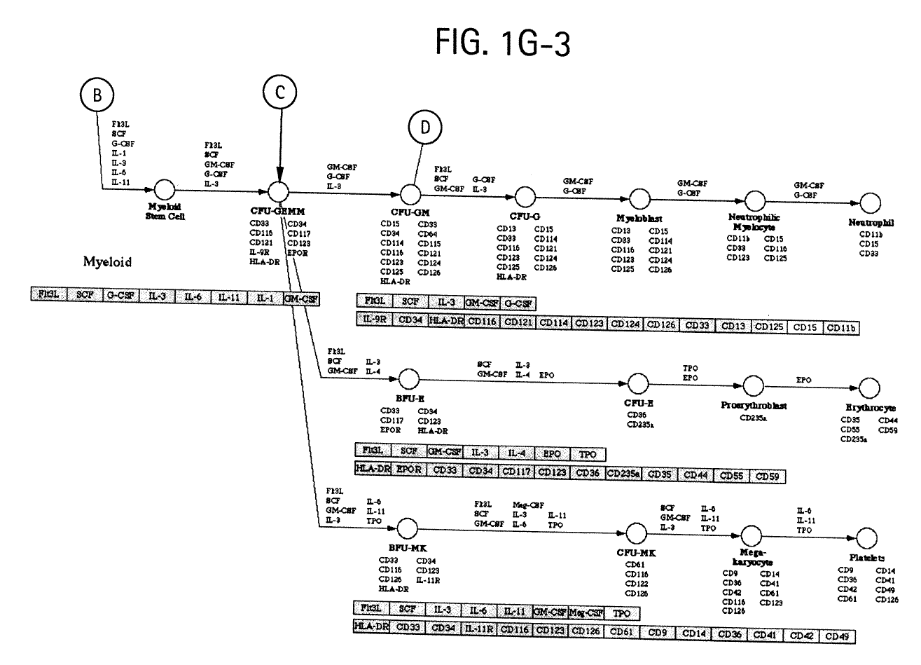

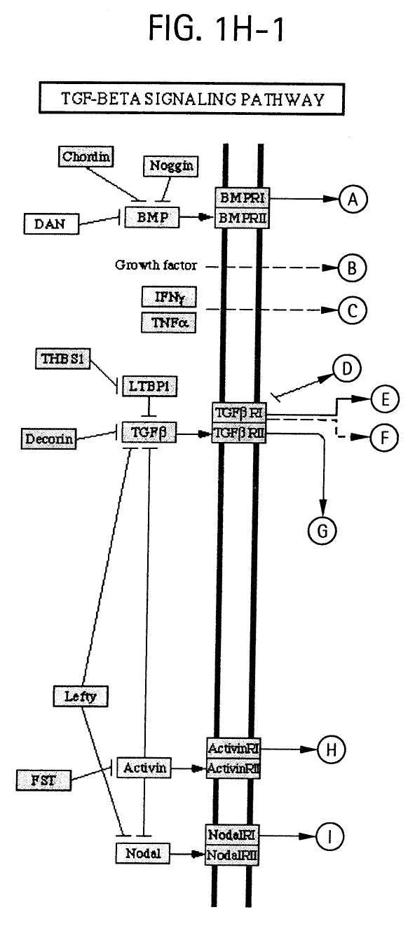

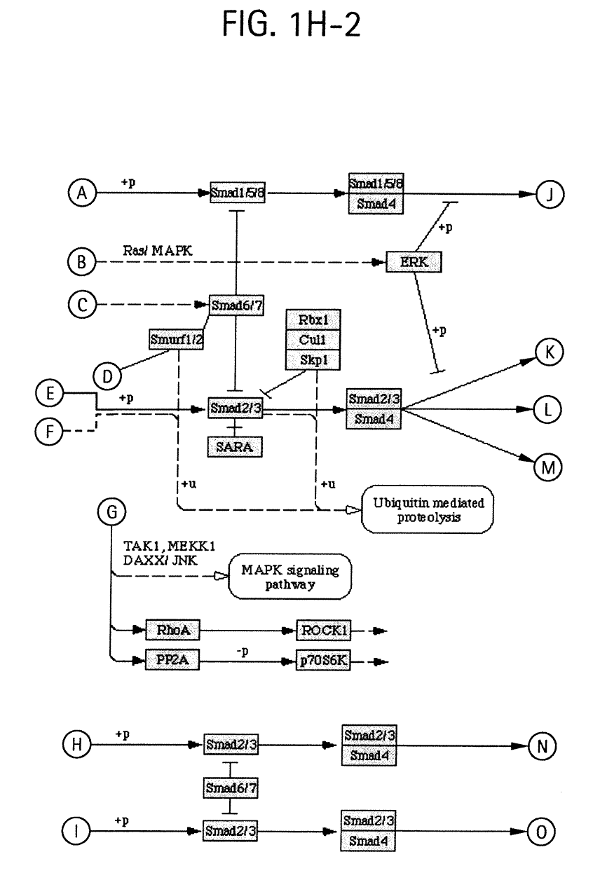

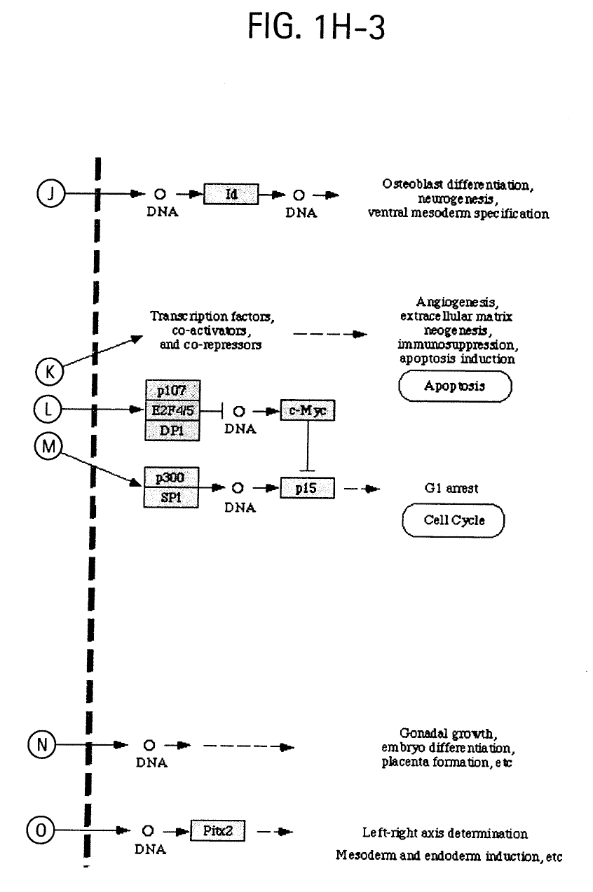

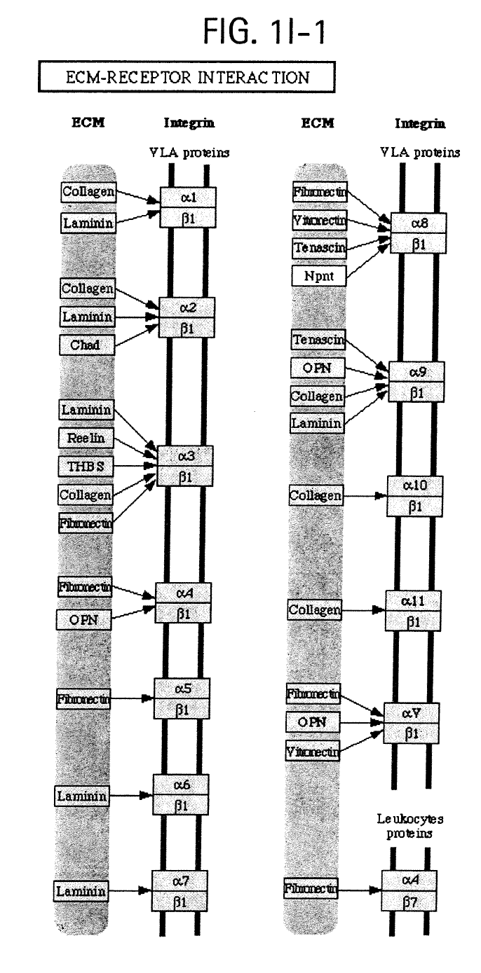

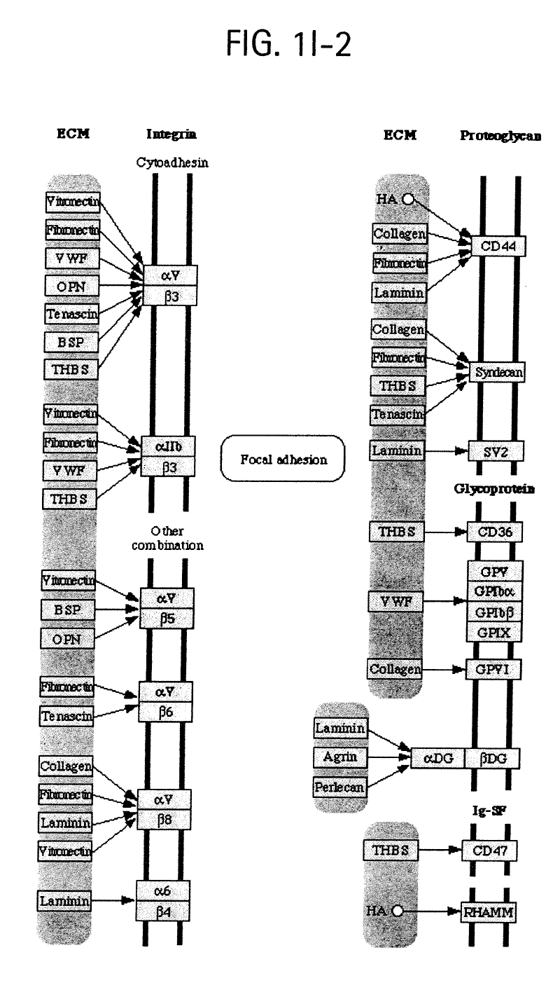

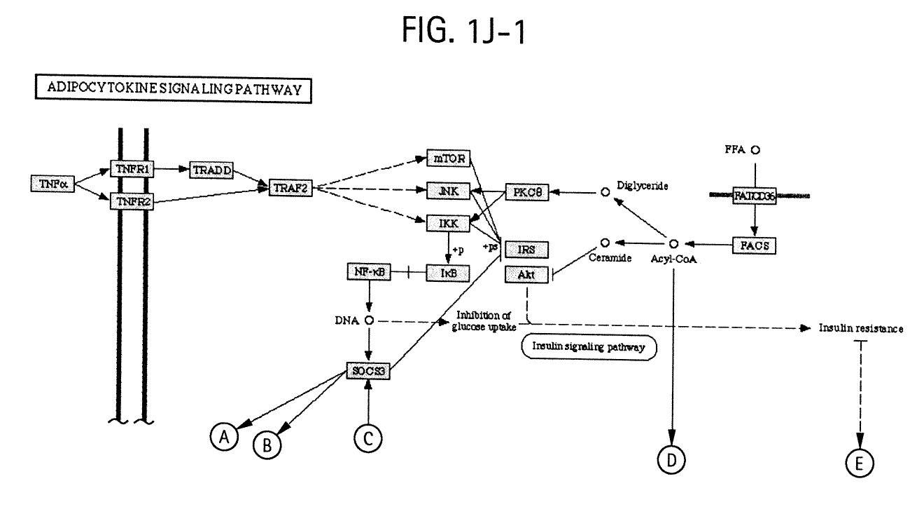

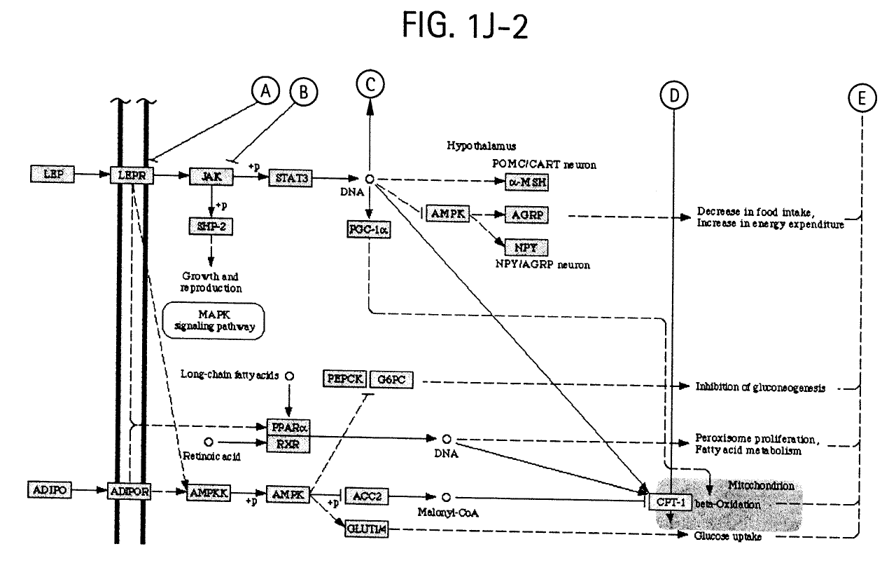

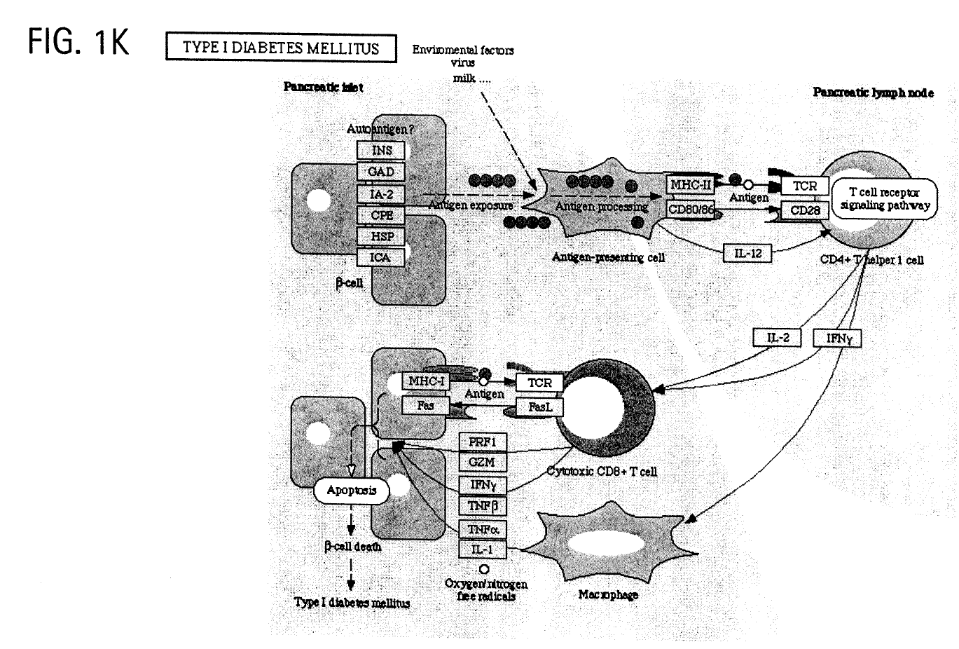

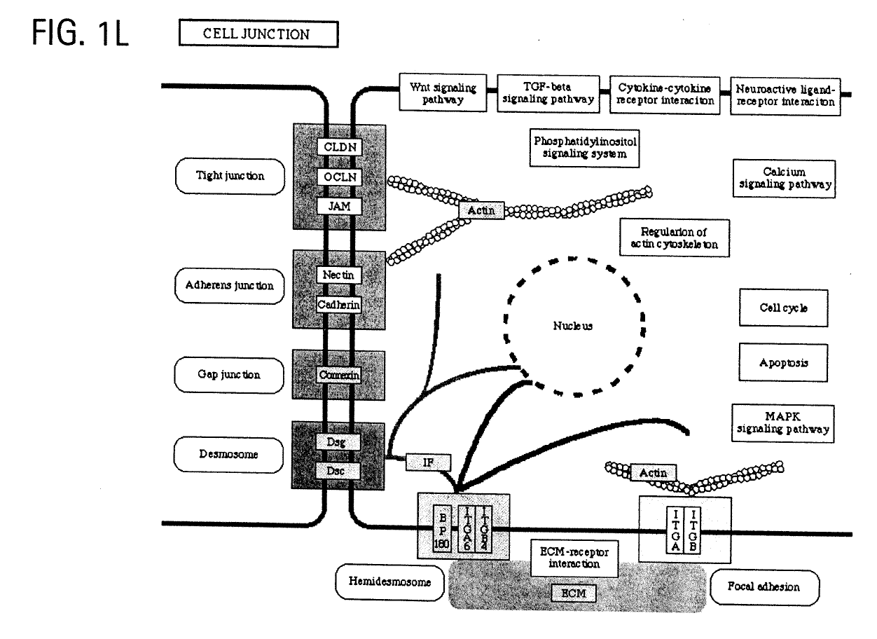

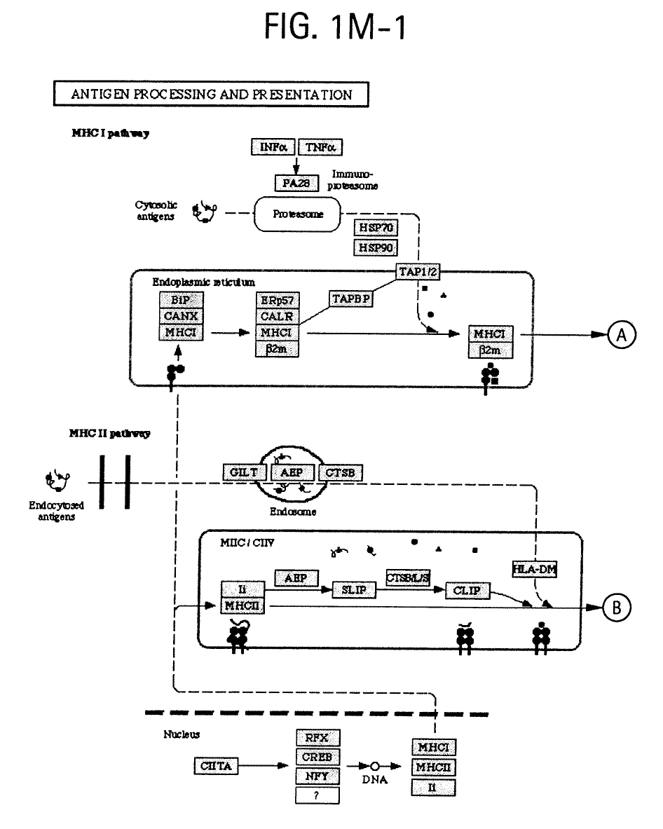

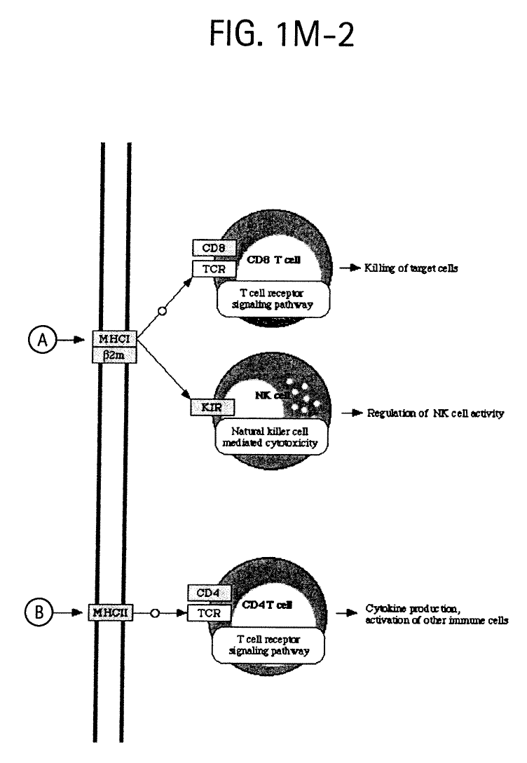

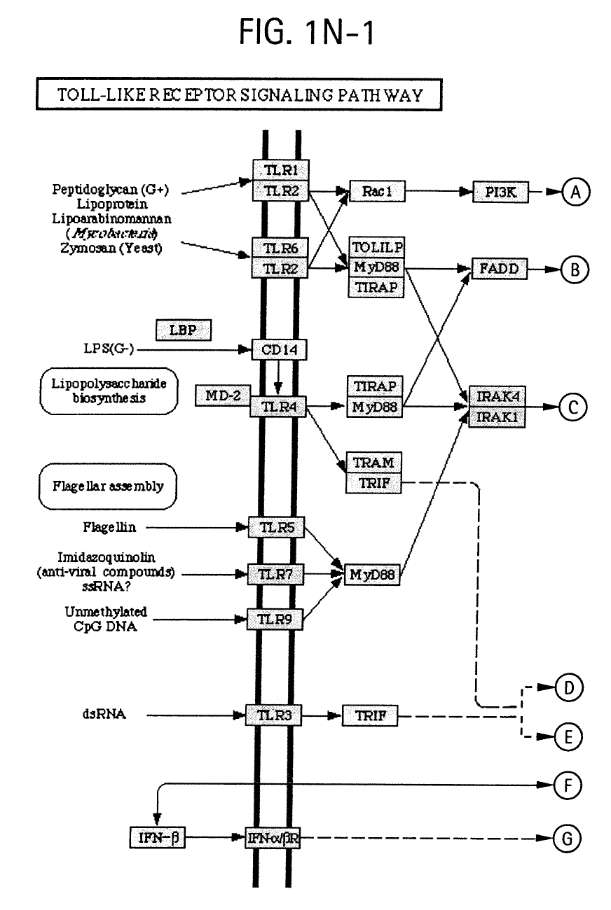

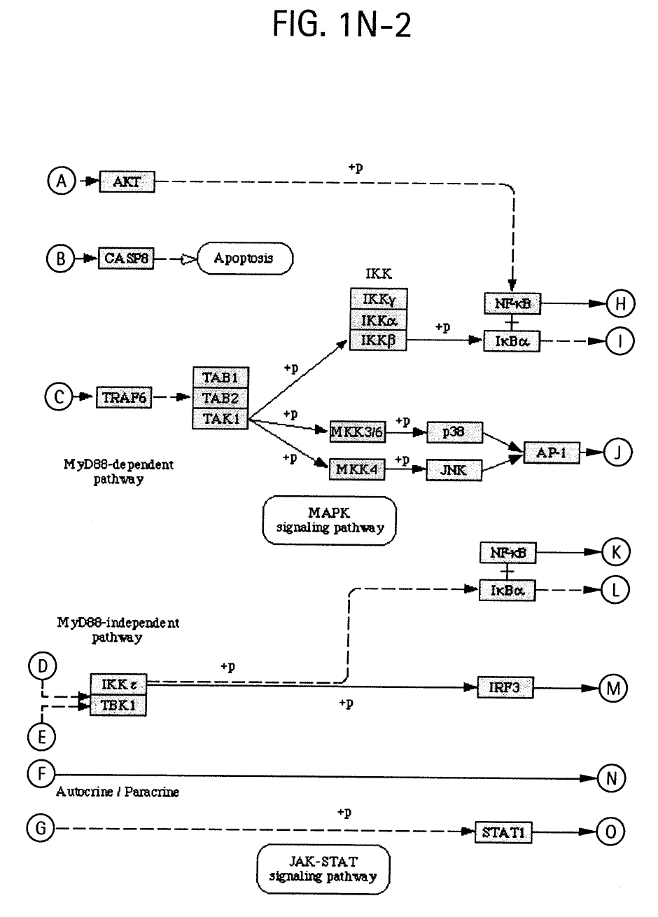

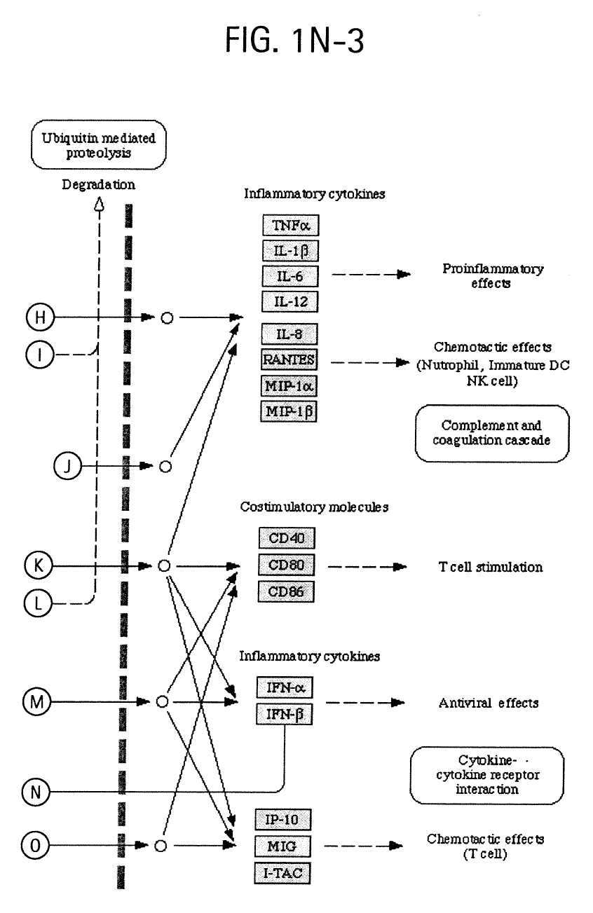

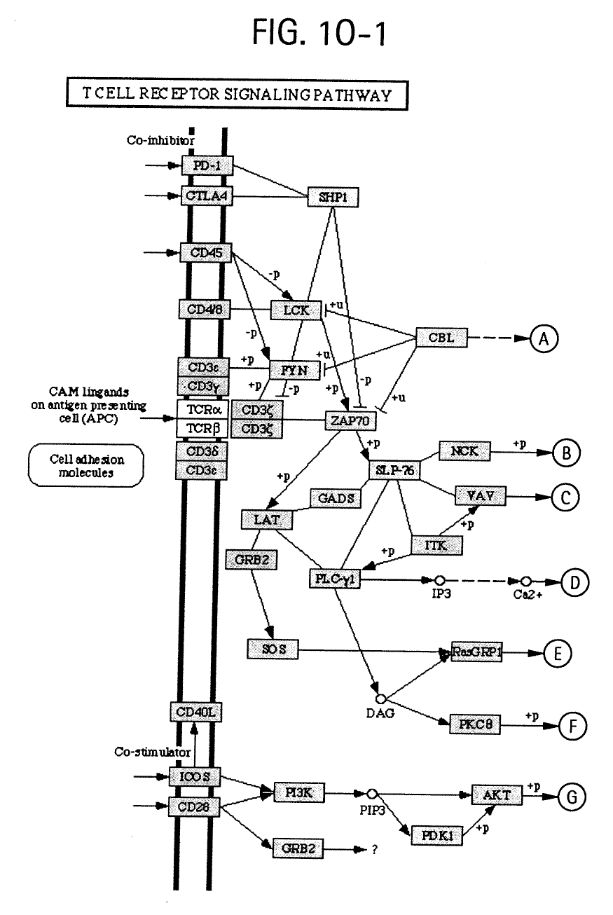

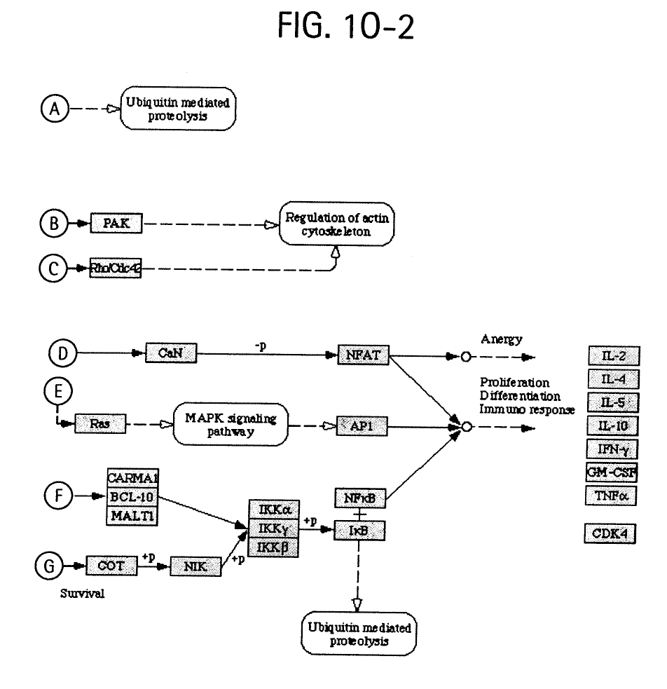

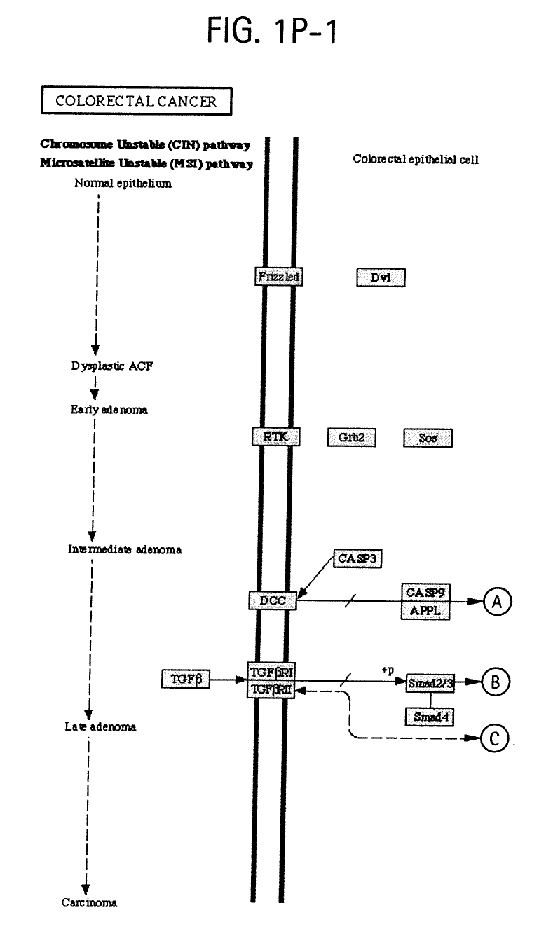

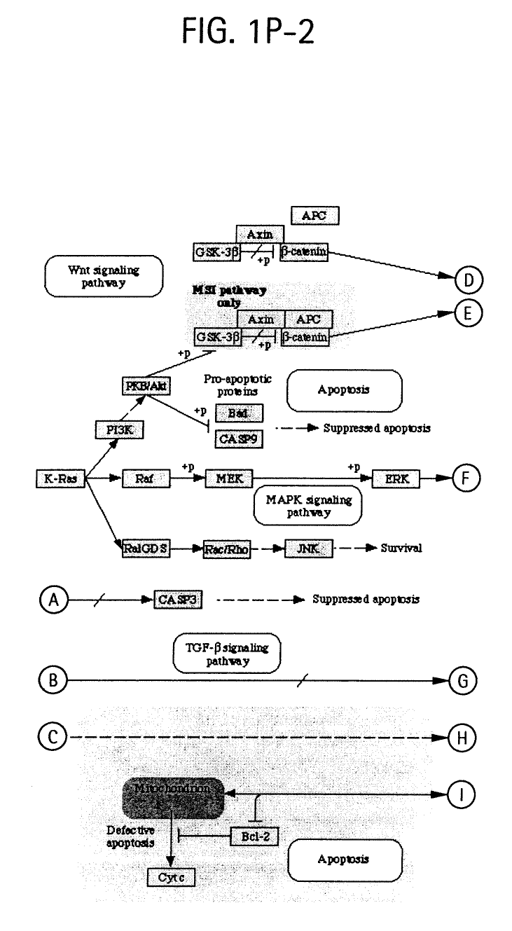

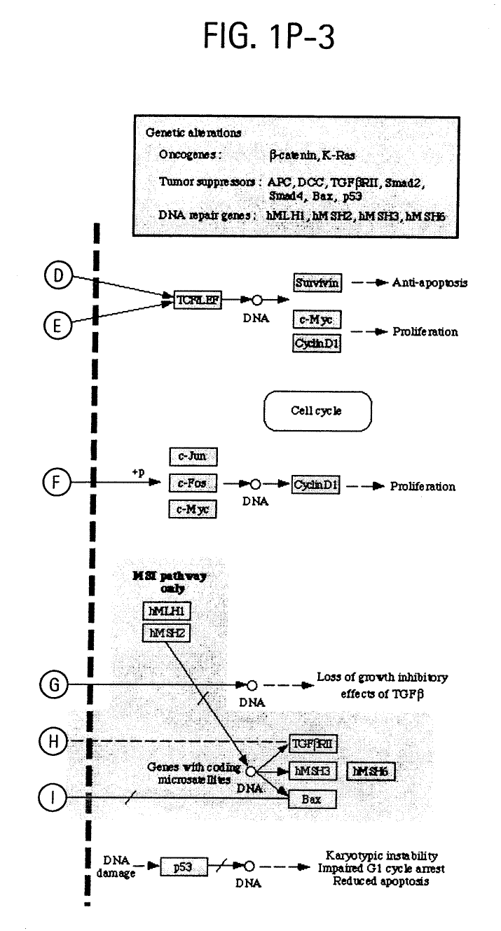

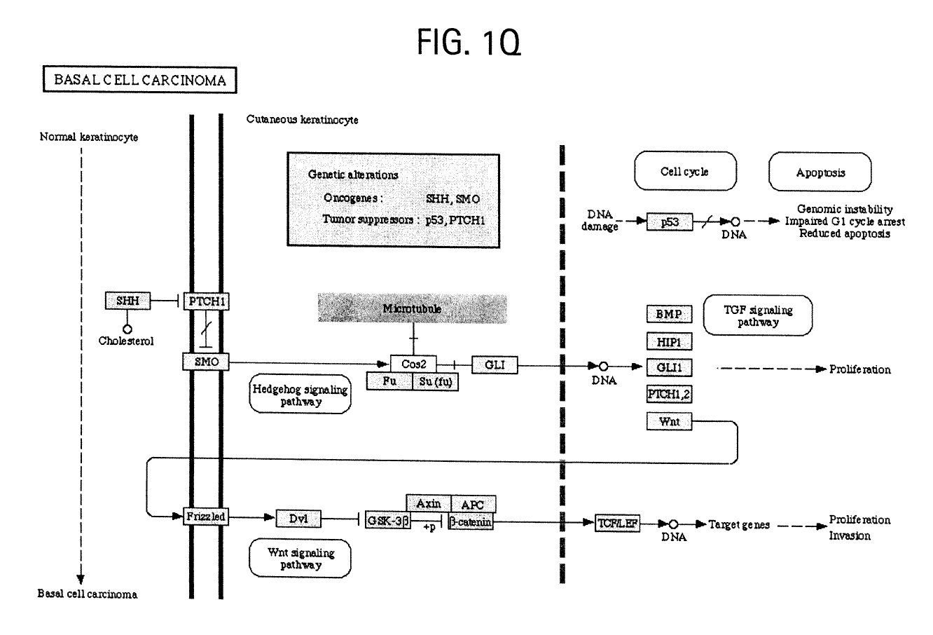

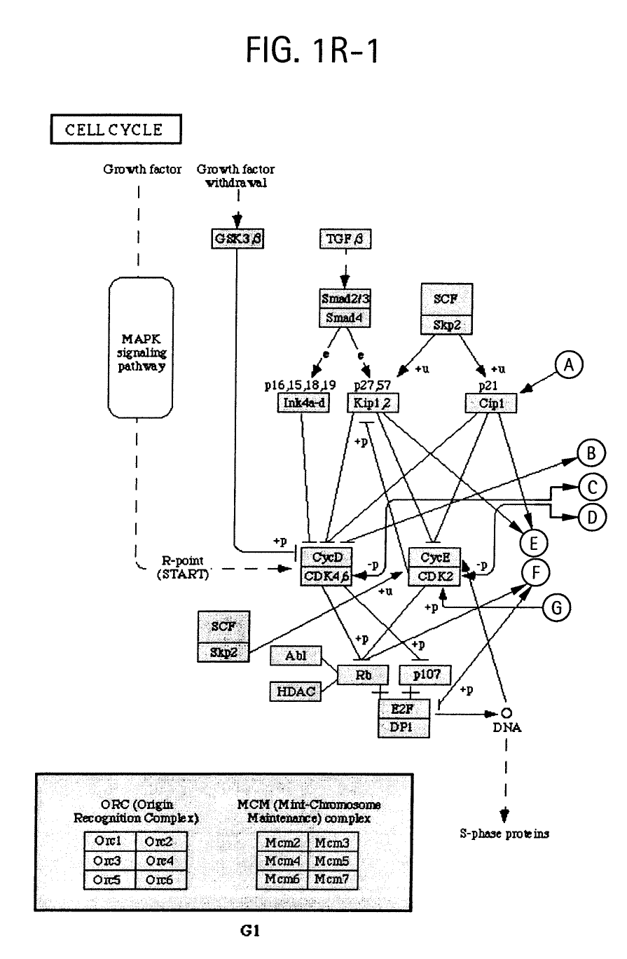

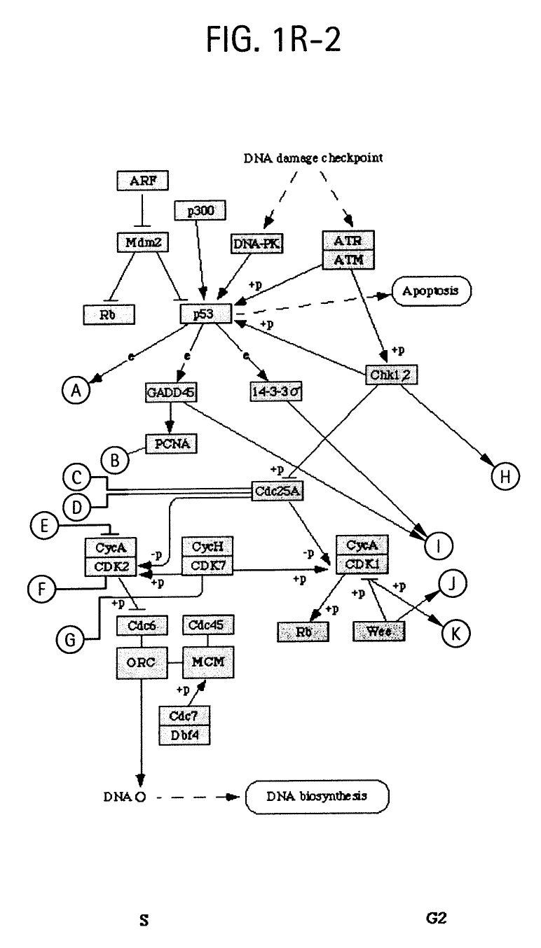

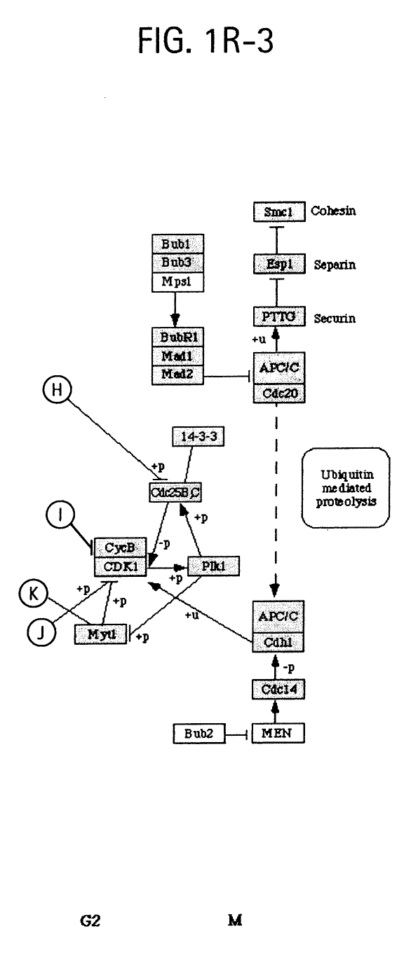

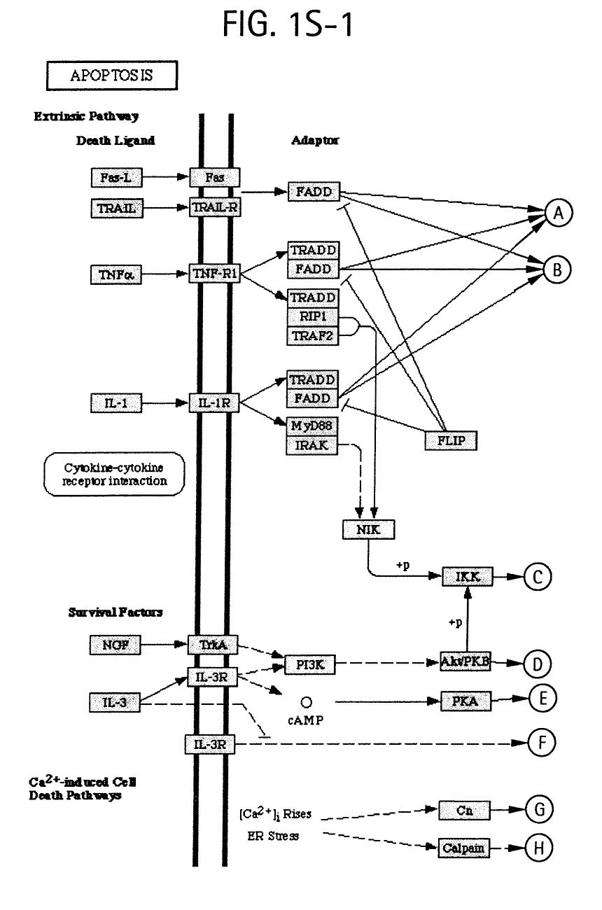

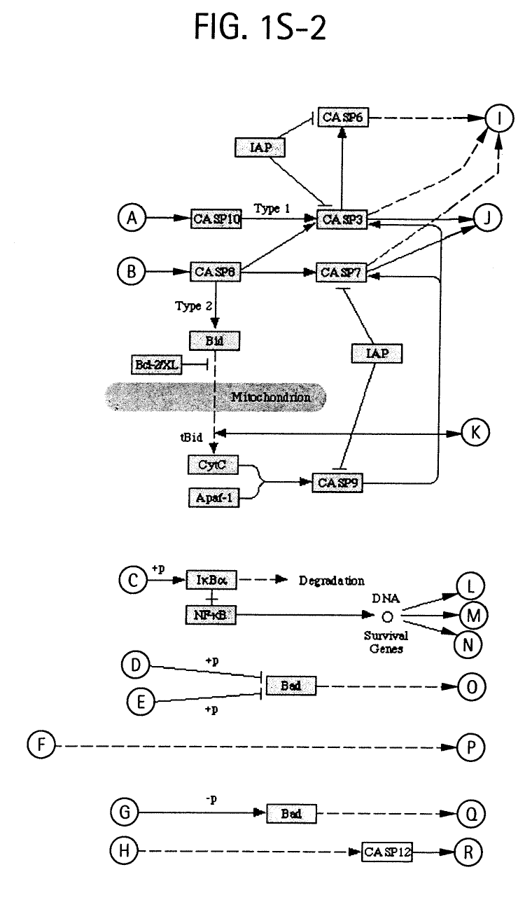

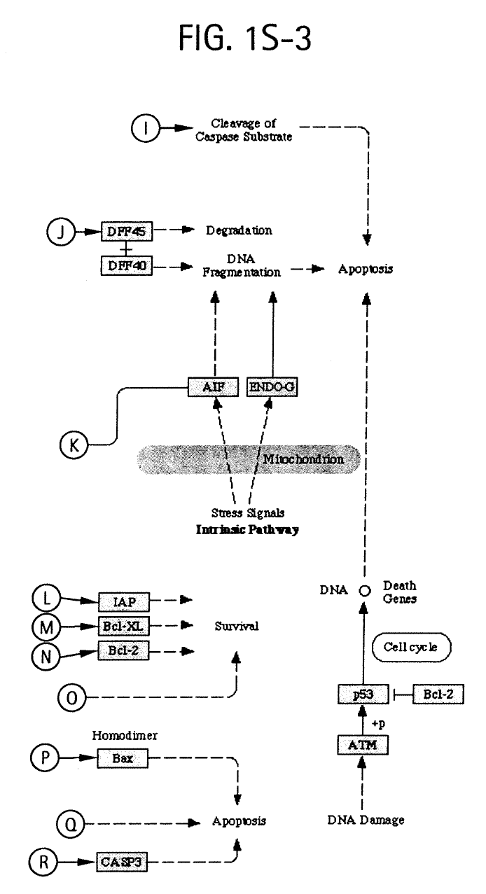

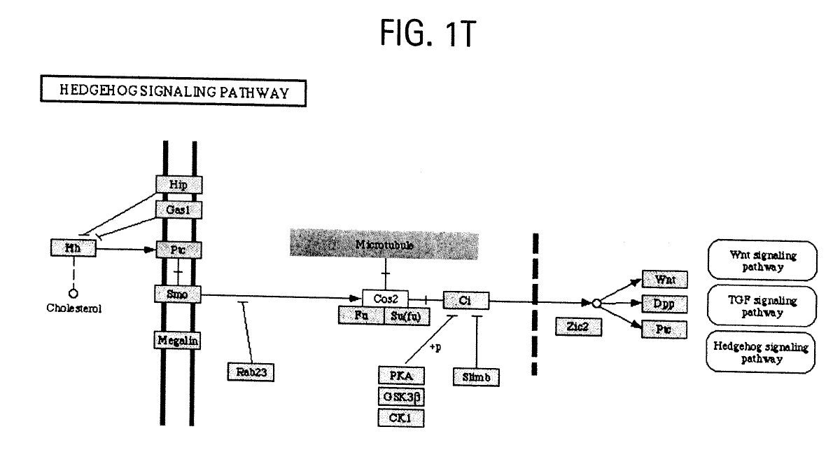

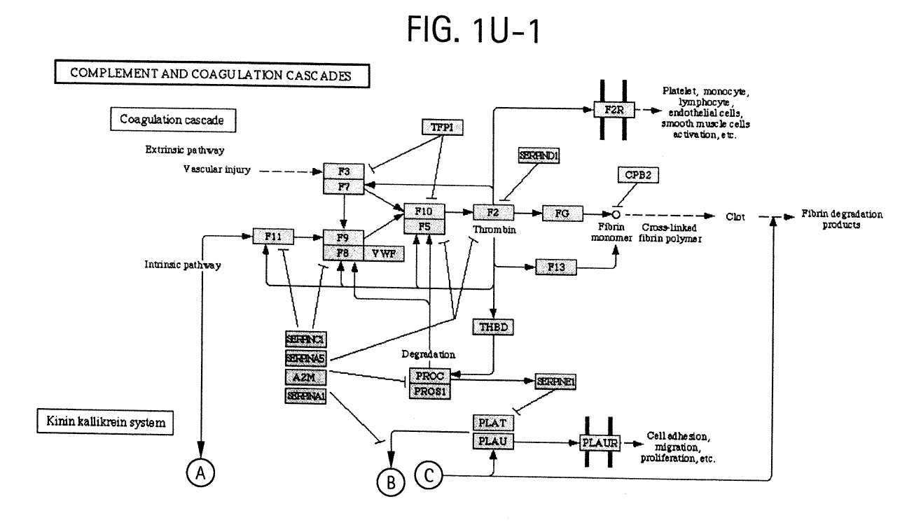

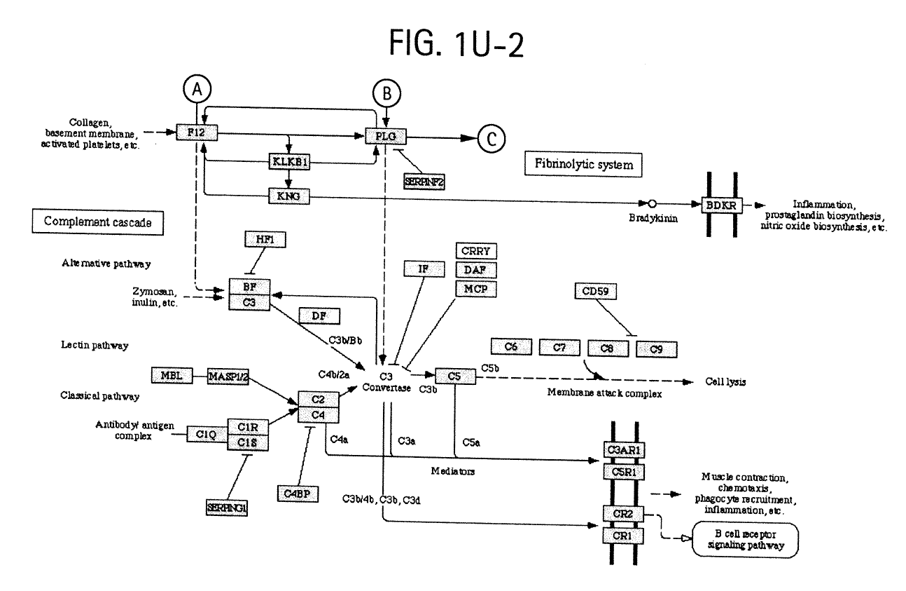

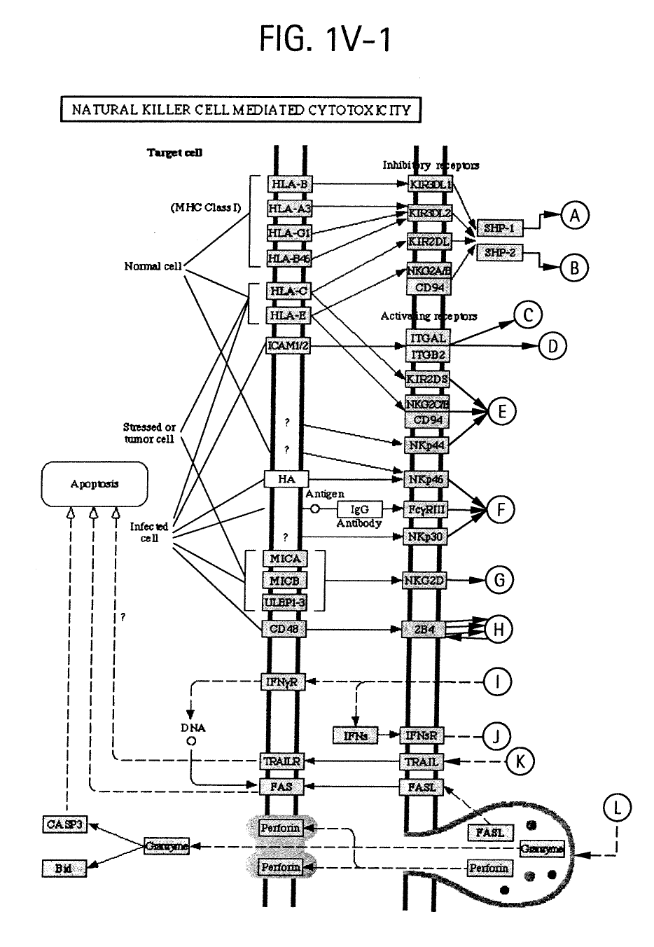

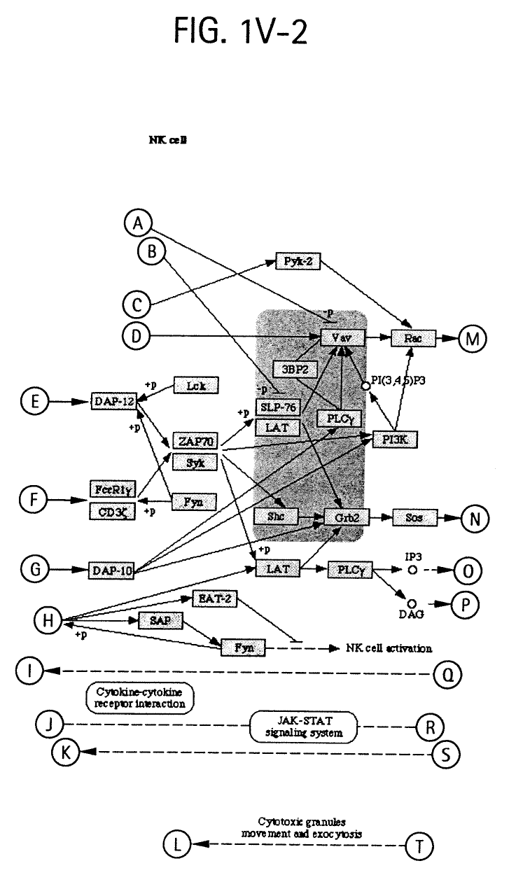

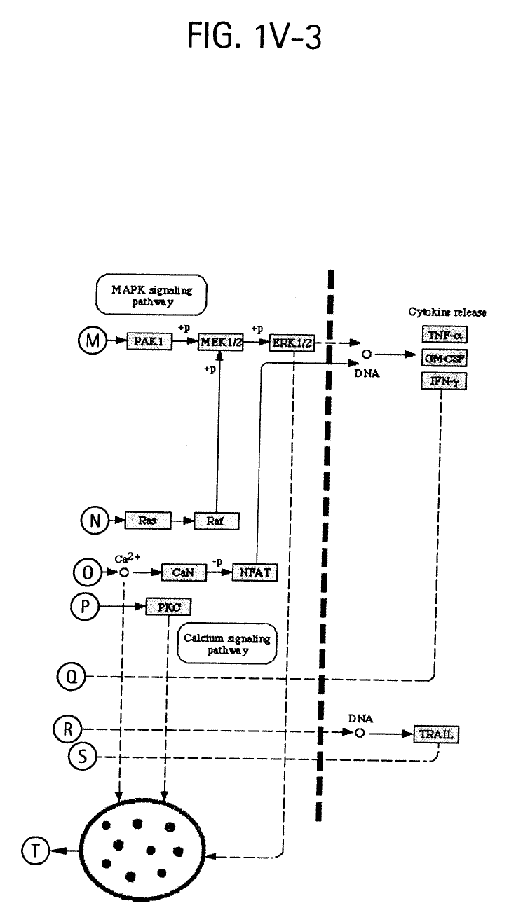

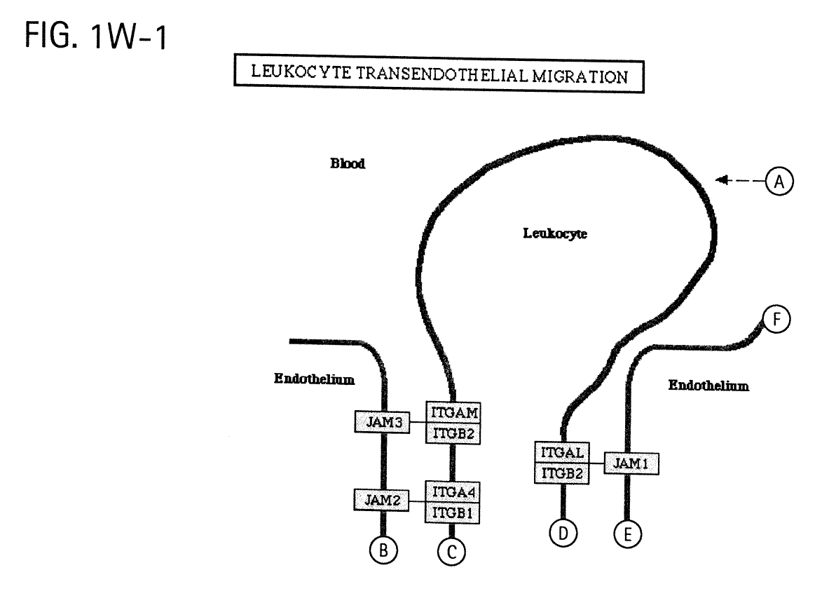

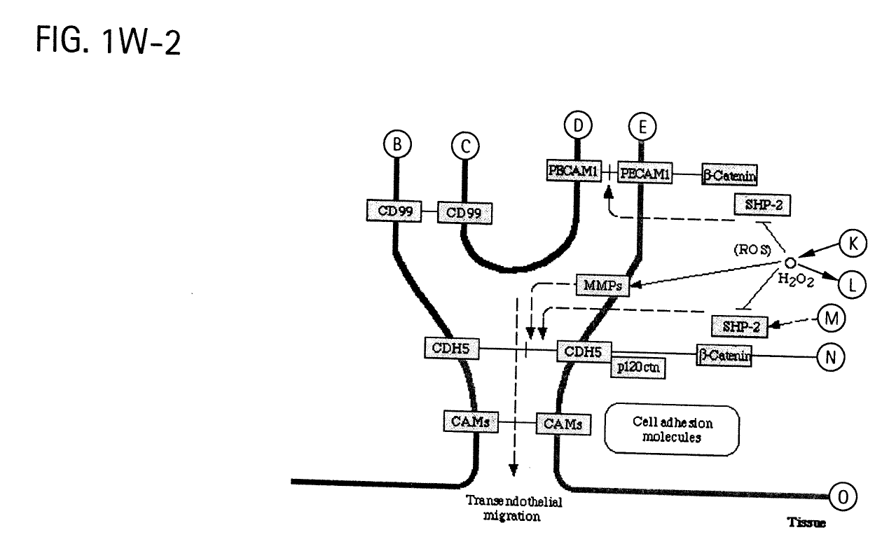

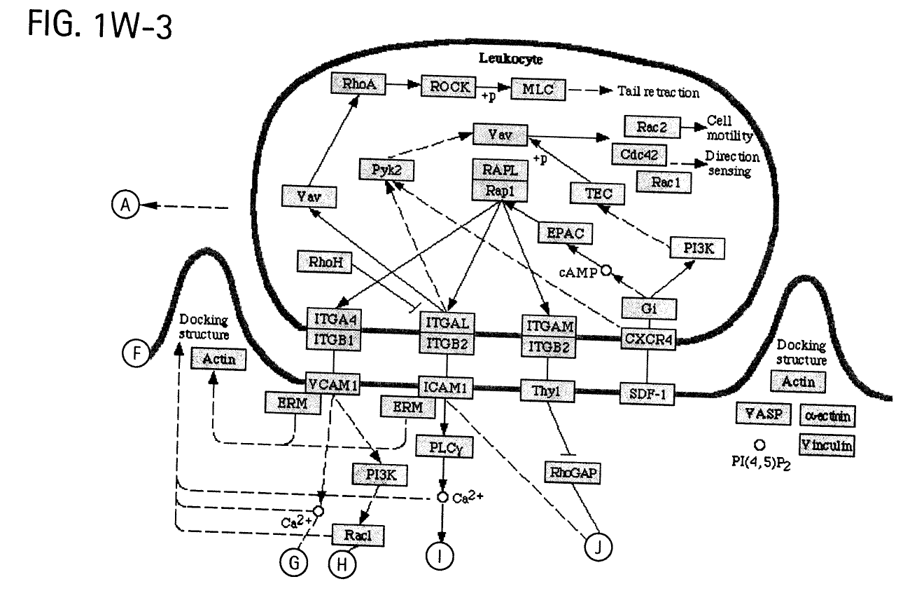

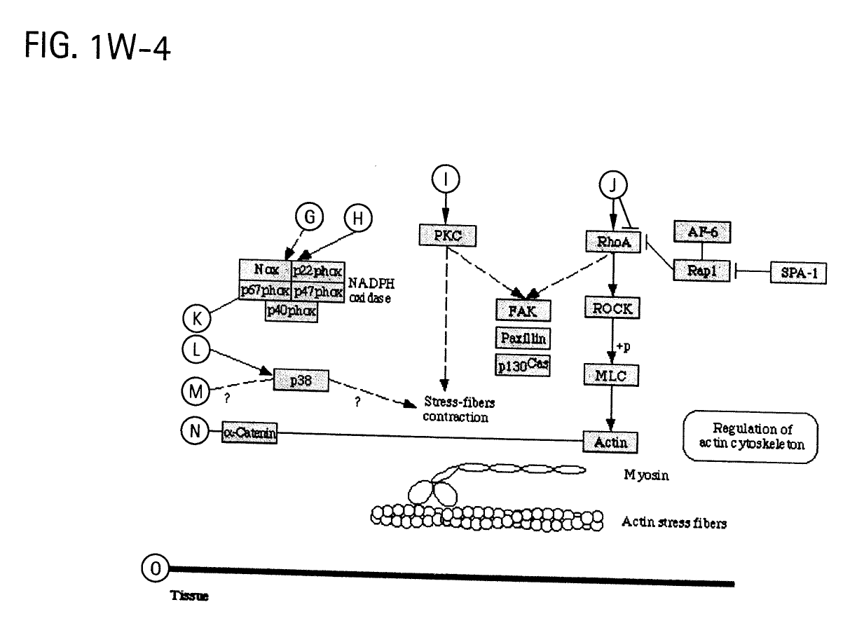

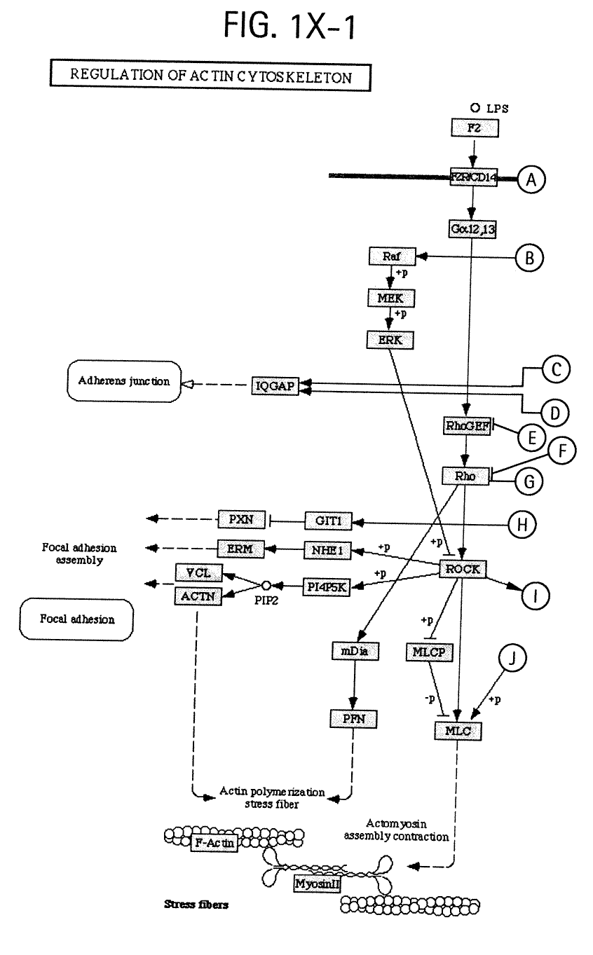

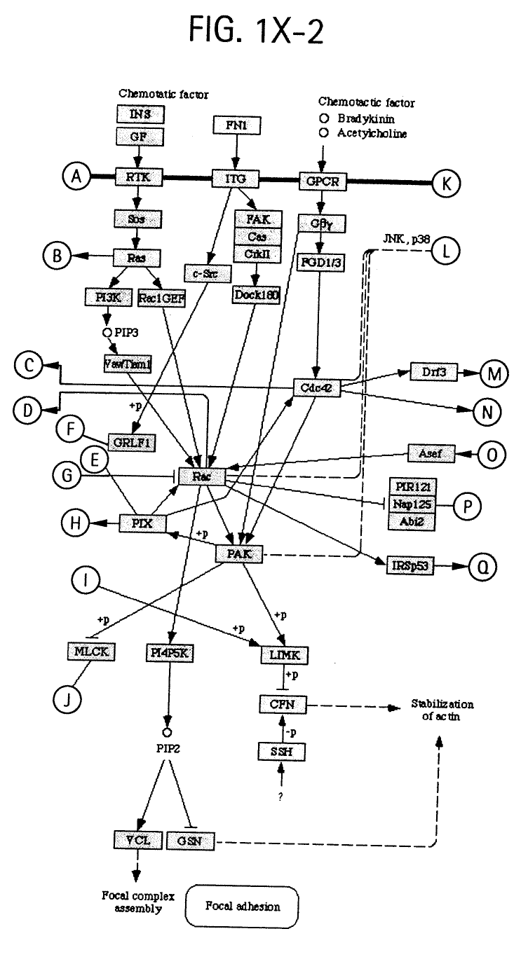

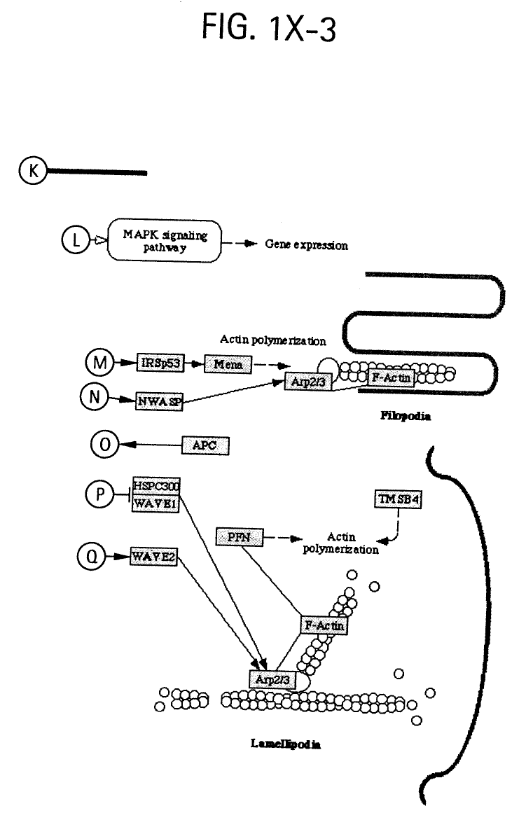

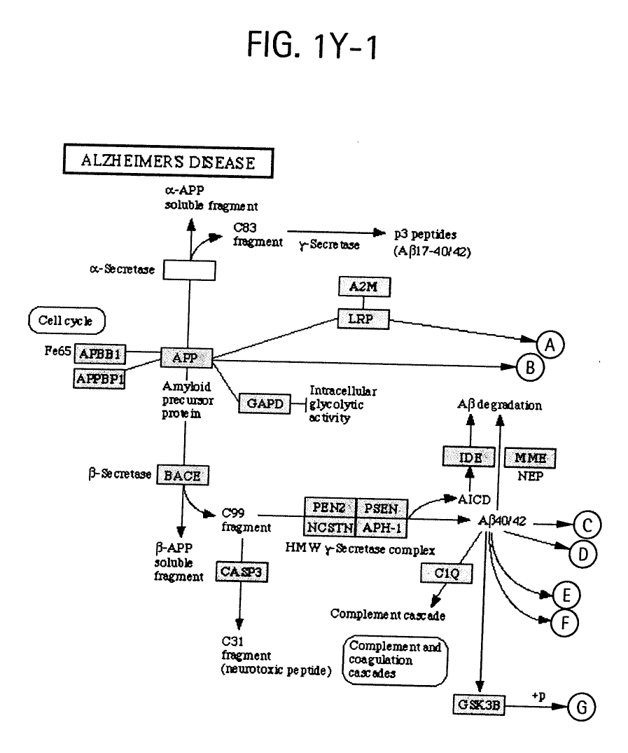

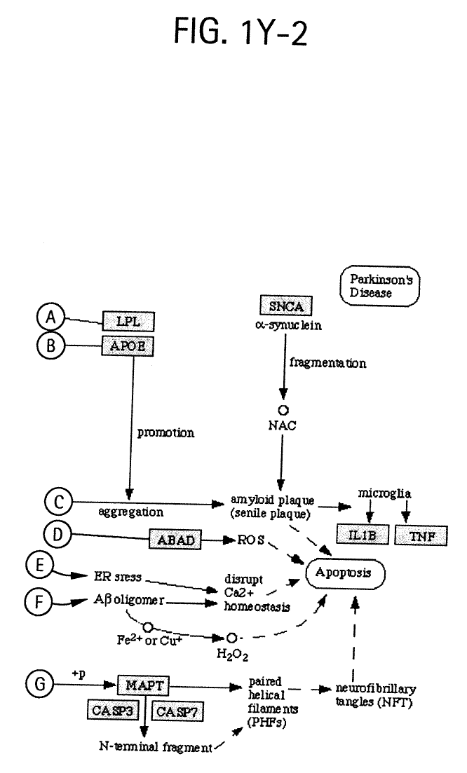

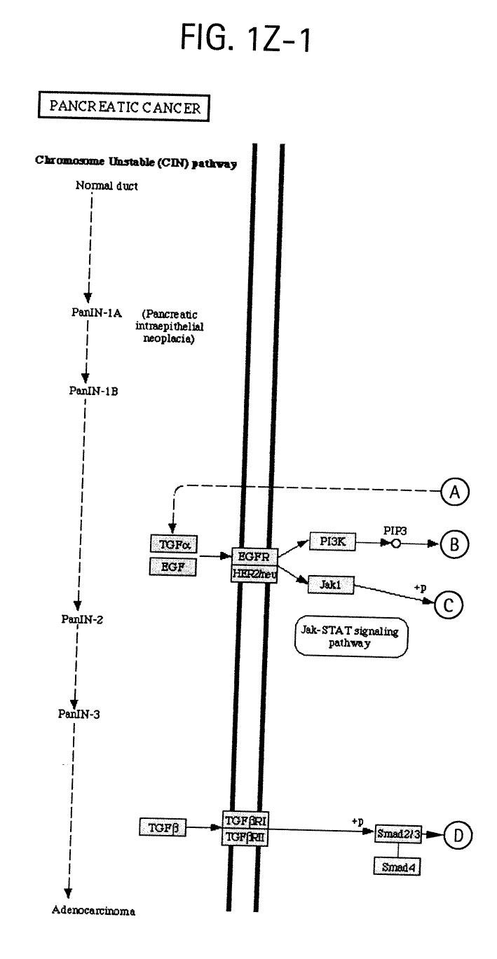

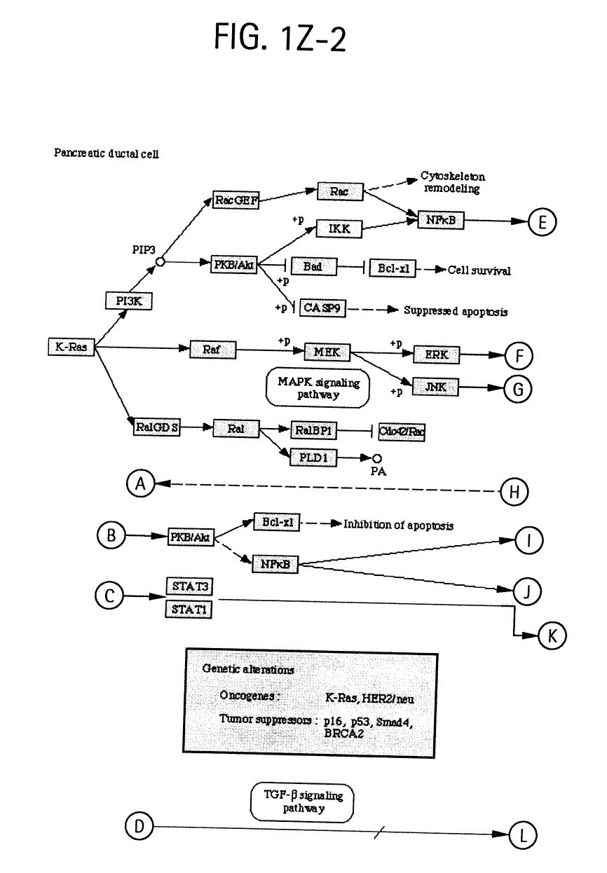

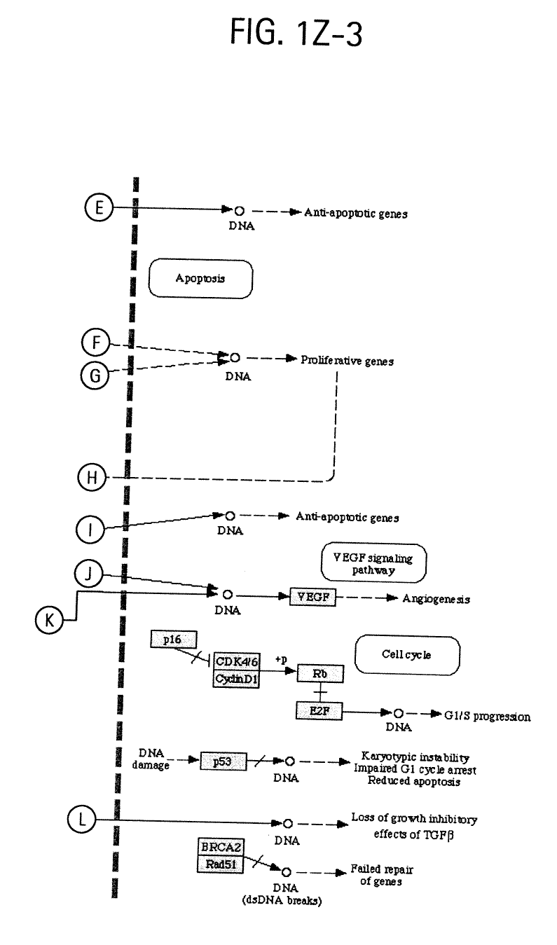

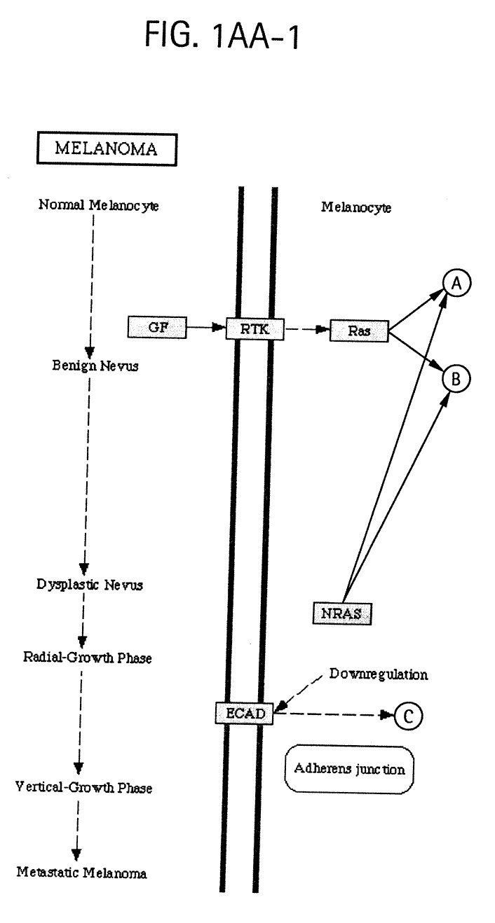

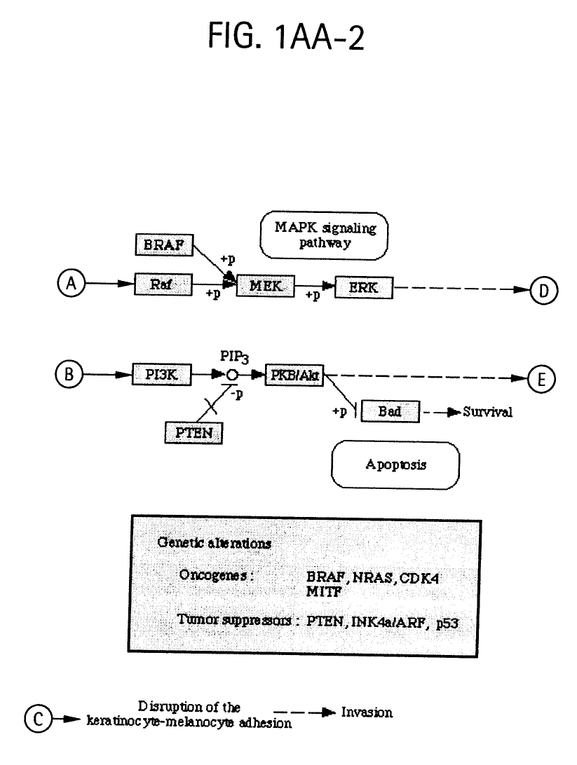

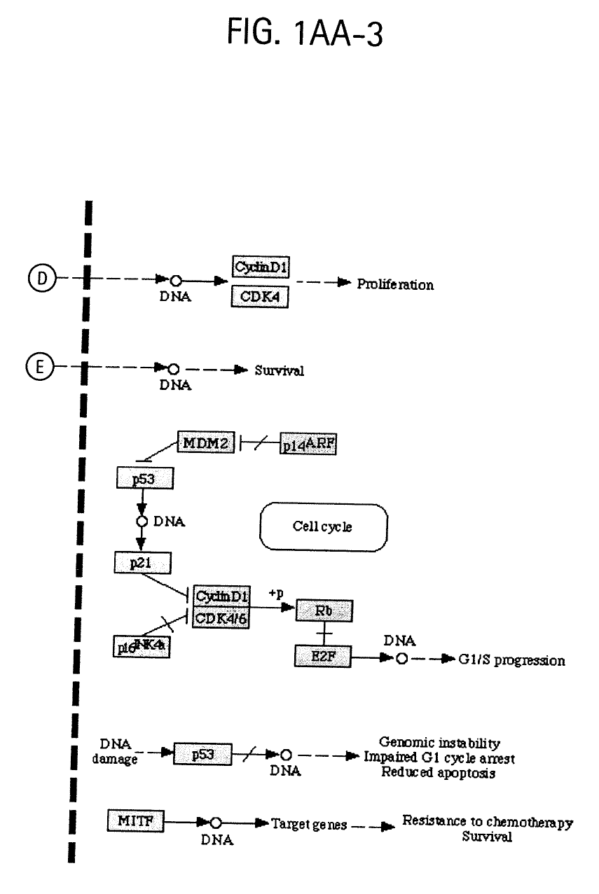

FIGS. 1P-1, 1P-2, and 1P-3 depict OSTEORISKMARKERS involved in colorectal cancer as shown in KEGG pathway hsa05210.

FIG. 1Q depicts OSTEORISKMARKERS involved in basal cell carcinoma as shown in KEGG pathway hsa05217.

FIGS. 1R-1, 1R-2, and 1R-3 depict OSTEORISKMARKERS involved in cell cycle interactions as shown in KEGG pathway hsa04110.

FIGS. 1S-1, 1S-2, and 1S-3 depict OSTEORISKMARKERS involved in apoptosis as shown in KEGG pathway hsa04210.

FIG. 1T depicts OSTEORISKMARKERS involved in Hedgehog signaling as shown in KEGG pathway hsa04340.

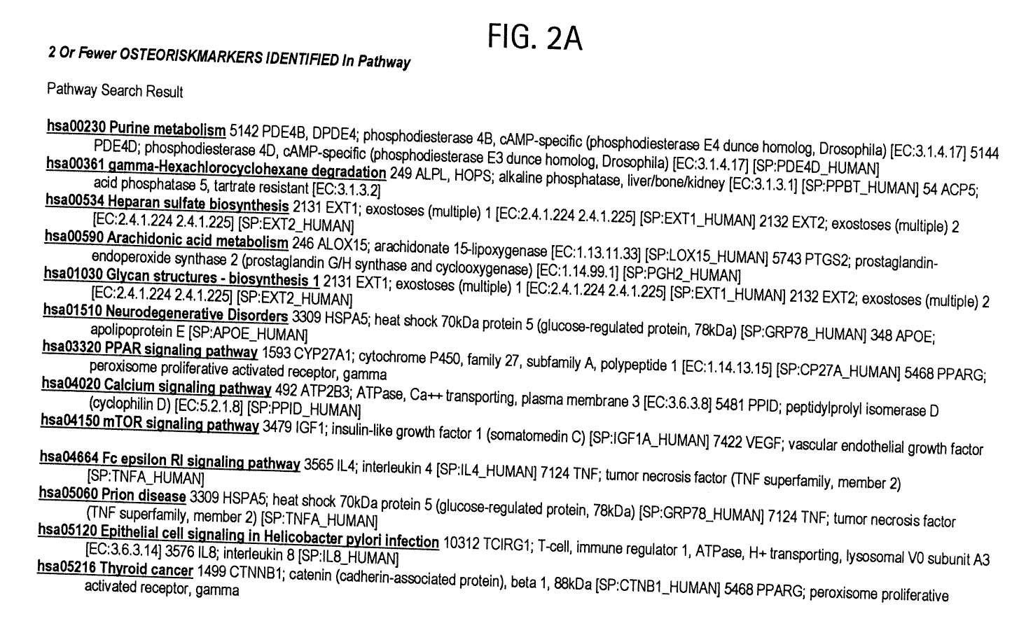

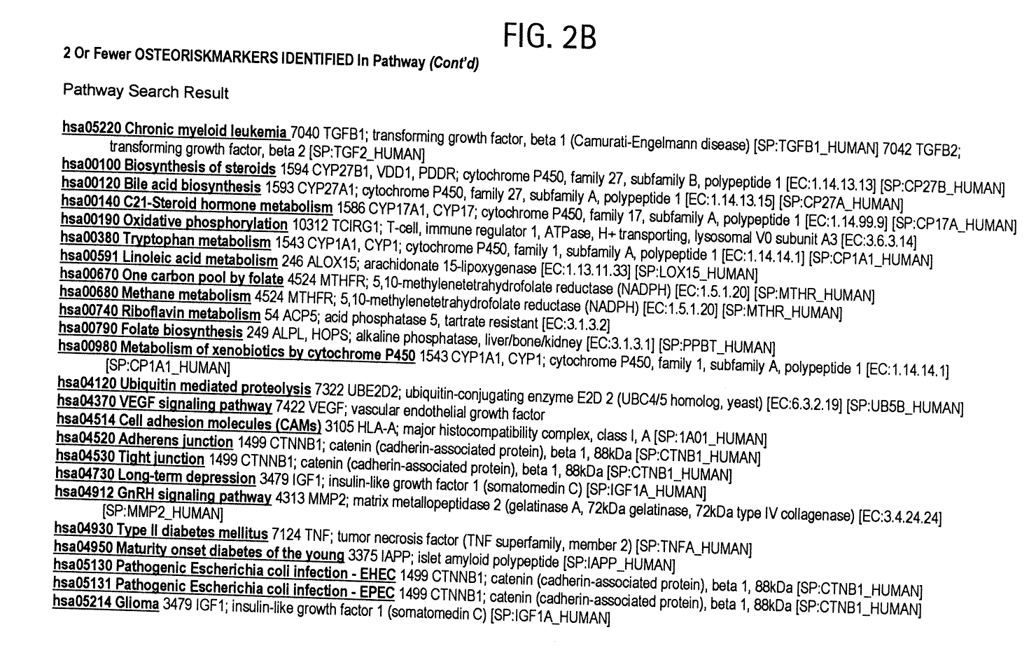

FIGS. 1U-1 and 1U-2 depict OSTEORISKMARKERS involved in complement and coagulation cascades as shown in KEGG pathway hsa04610.

FIGS. 1V-1, 1V-2, and 1V-3 show OSTEORISKMARKERS involved in natural killer cell-mediated cytotoxicity as depicted in KEGG pathway hsa04650.

FIGS. 1W-1, 1W-2, 1W-3, and 1W-4 show OSTEORISKMARKERS involved in leukocyte transendothelial migration as depicted in KEGG pathway hsa04670.

FIGS. 1X-1, 1X-2, and 1X-3 show OSTEORISKMARKERS involved in regulation of the actin cytoskeleton as depicted in KEGG pathway hsa04810.

FIGS. 1Y-1 and 1Y-2 show OSTEORISKMARKERS involved in Alzheimer's Disease as depicted in KEGG pathway hsa05010.

FIGS. 1Z-1, 1Z-2, and 1Z-3 show OSTEORISKMARKERS involved in pancreatic cancer as depicted in KEGG pathway hsa05212.

FIGS. 1AA, 1AA-2, and 1AA-3 show OSTEORISKMARKERS involved in melanoma as depicted in KEGG pathway hsa05218.

FIGS. 2A and 2B represent a listing of KEGG pathways with one or two OSTEORISKMARKERS identified as contained within them.

FIGS. 3-1 and 3-2 are tables listing individual OSTEORISKMARKERS divided into general categories based on their associations with the physiological functions of bone formation (left column) and of bone resorption (right column). OSTEORISKMARKERS which are commonly found localized in the extracellular space or plasma membranes of cells are also highlighted in bold or italics, respectively, in this and the following Figures.

FIGS. 4-1, 4-2, and 4-3 are tables listing additional individual OSTEORISKMARKERS categorized by their association with the following physiological functions and/or categories: osteoclast metabolism (category A), osteocyte metabolism (category B), osteoblast metabolism (category C), calcium metabolism (category D), bone ossification or mineralization (category E), skeletal development (category F), muscle cell metabolism (including the proliferation and movement of muscle cells, including vascular and vascular smooth muscle cells; category G), eicosanoid metabolism (category H), other metabolism (category I), and other bone-related physiology (category J).

FIGS. 5-1, 5-2, 5-3, 5-4, and 5-5 are tables listing various combinations useful in constructing panels of the additional OSTEORISKMARKERS from FIGS. 4-1 through 4-3, indicating the use of one or more markers each from one or more of the previously mentioned categories, constructed according to the invention. In one embodiment of the invention, these additional OSTEORISKMARKER combination panels may themselves be further combined with one or more OSTEORISKMARKER(S) selected from either one or both of the general categories of bone formation and of bone resorption, respectively, previously identified in FIGS. 3-1 and 3-2.

FIGS. 6-1 and 6-2 are tables listing eleven clusters of OSTEORISKMARKERS grouped by their relative position, interactions, and network proximity as defined by protein-protein interactions and through participation in one or more canonical pathways, presented in the figure together with their near neighbors and interaction partners within pathways. OSTEORISKMARKER panels may also be constructed by means of selection of one or more OSTEORISKMARKERS each from one or more of the eleven clusters listed. Such OSTEORISKMARKERS may be further selected by virtue of their cell localization. OSTEORISKMARKERS which are commonly found localized in the extracellular space or plasma membranes of cells are also highlighted in bold or italics, respectively.

### DETAILED DESCRIPTION OF THE INVENTION

The present invention relates to the identification of biomarkers associated with subjects having bone metabolic disorders such as osteoporosis and osteopenia, or are predisposed to or at risk for developing osteoporosis, osteopenia, or bone fractures. Accordingly, the invention provides methods for identifying subjects who have osteoporosis or osteopenia, or who are predisposed to or at risk for developing osteoporosis, osteopenia, or bone fractures by the detection of biomarkers associated with same. These biomarkers are also useful for monitoring subjects undergoing treatments and therapies for osteoporosis, osteopenia, or bone fractures, and for selecting therapies and treatments that would be efficacious in subjects having osteoporosis, osteopenia, or bone fractures, wherein selection and use of such treatments and therapies slow the progression of osteoporosis or osteopenia, or substantially delay or prevent their onset.

“Osteoporosis” is defined in the art as a systemic skeletal disease characterized by low bone mass and microarchitectural deterioration of bone tissue, with a consequent increase in bone fragility and susceptibility to fracture. Any bone can be affected by osteoporosis, although the hip, spine, and wrist are common bones that are broken or fractured in subjects suffering from or at risk for osteoporosis.

Osteoporosis in postmenopausal Caucasian women is defined as a value for bone mineral density (BMD) of >2.5 SD below the young average value, i.e. a T-score of 2.5 SD. Severe osteoporosis (established osteoporosis) uses the same threshold, but with one or more prior fragility fractures. The preferred site for diagnostic purposes are BMD measurements made at the hip, either at the total hip or the femoral neck. For men, the same threshold as utilized for women is appropriate, since for any given BMD, the age adjusted fracture risk is more or less the same.

“Osteopenia” is a pre-osteoporosis condition characterized as a mild thinning of bone mass which is not as severe as osteoporosis. Osteopenia results when the formation of bone is not enough to offset normal bone loss. Osteopenia is generally considered the first step along the road to osteoporosis. Diminished bone calcification can also be referred to as osteopenia, whether or not osteoporosis is present.

“Pre-Osteoporosis” encompasses both osteopenia and also other conditions which result in a high risk of future development of osteopenia, osteoporosis, and bone fracture. Subjects who are deemed clinically to be at low risk or no risk for developing osteoporosis or osteopenia based on current BMD nevertheless may still be at risk for pre-osteoporosis or bone fracture, as BMD measures bone status at the time of assessment and not rate of bone metabolism or predisposition to a lowered future BMD. The majority of bone fractures occur in subjects who have not been previously diagnosed with osteoporosis or pre-osteoporosis. There is a substantial need for better risk assessment and stratification tools for those who do not yet have osteoporosis or osteopenia yet are expected to have higher than normal rates of progression to those symptomatic disease states measurable by BMD.

The diagnostic threshold set forth by WHO identifies approximately 20% of postmenopausal women as having osteoporosis when measurements using dual energy X-ray absorptiometry (DXA) are made at the hip. The diagnostic use of the T-score cannot be used interchangeably with different techniques and at different sites, since the same T-score derived from different sites and techniques yields different information on fracture risk. For example, in women at the age of 60 years the average T-score ranges from −0.7 to −2.5 SD, depending on the technique used. Reasons include differences in the gradient of risk with which techniques predict fracture, discrepancies in the population standard deviation, and differences in the apparent rates of site-specific bone loss with age. A further problem is that inter-site correlations, although usually of statistical significance, are inadequate for predictive purposes in individuals giving rise to errors of mis-classification.

The cornerstone for the diagnosis of osteoporosis lies in the assessment of BMD (See Kanis et al., Assessment of Fracture Risk, Osteoporosis International (2005) 16: 581-589). BMD should be recognized as assessing the bone mineral density at a point in time, and requires repeat testing in order to monitor changes in density; density alone is a relatively slow indicator of changes in bone. The same T-score with the same technique at any one site has a different significance at different ages. For any given T-score, fracture risk is much higher in the elderly than in the young, because age contributes to risk independently of BMD. BMD also suffers from several disadvantages in its requirement for specialized equipment and expertise. The use of bone mass measurements for prognosis (risk assessment) depends upon accuracy. Accuracy in this context is the ability of the measurement to predict fracture. In general, all absorptiometric techniques have high specificity but low sensitivity that varies with the cut-off chosen to designate high risk.

Fracture risk is commonly expressed as a relative risk, but this has different meanings in different contexts. In the case of bone density measurements, gradients of risk are used, e.g. a 2.6-fold increase in hip fracture risk for each SD decrease in BMD. For dichotomous risk factors, risk is commonly expressed as the risk in individuals with a risk factor compared to the risk in those without the risk factor, or, as a risk compared with the general population.

The absolute risk of fracture depends upon age and life expectancy as well as the current relative risk. In general, remaining lifetime risk of fracture increases with age up to the age of 70 years or so. Thereafter, probability plateaus and then decreases, since the risk of death with age outstrips the increasing incidence of fracture with age. Estimates of lifetime risk are of value in considering the burden of osteoporosis in the community, and the effects of intervention strategies. For several reasons, they are less relevant for assessing risk of individuals in whom treatment might be envisaged. Firstly, treatments are not presently given for a lifetime, due variably to side effects of continued treatment (e.g. hormone replacement treatment) or low continuance (most treatments). Moreover, the feasibility of life-long interventions has never been tested, either using high risk or global strategies. Secondly, the predictive value of low bone mineral density and some other risk factors for fracture risk may be attenuated over time. Finally, the confidence in estimates decreases with time due to the uncertainties concerning future mortality trends. Risk of fracture should be expressed as a fixed-term absolute risk, i.e. probability over a 10-year interval. The period of 10 years covers the likely duration of treatment and any benefits that may continue once treatment is stopped.

Other than direct measurement of BMD, several conventional risk factors for osteoporosis and bone fracture are often assessed prior to or in parallel with a diagnosis of osteoporosis or assessment of pre-osteoporosis conditions. Such risk factors include, without limitation, gender, wherein the chances of developing osteoporosis or osteopenia are greater in females due to less bone tissue as well as changes that happen during menopause; age, wherein bones become thinner and weaker with age; small body size; ethnicity, wherein Caucasian and Asian women are at highest risk and African American and Hispanic women have a lower but significant risk; family history, wherein fracture risk is thought to be due, in part, to genetics. Subjects whose parents have a history of fractures are reported to also have reduced bone mass and may be at risk for fractures.

Other significant risk factors include abnormally low levels of sex hormones, indicated by the abnormal absence of menstrual periods (amenorrhea), low estrogen levels such as found during female menopause (including, without limitation, low levels of any one or more of the primary estrogens, estradiol, estriol, and estrone, and their intermediates, precursor androgens and estrogen derivatives), and low testosterone level such as found in older men. Subjects suffering from anorexia nervosa are also at increased risk for osteoporosis. Diets low in calcium and vitamin D can also result in a higher incidence of bone loss. Subjects who undergo long-term use of glucocorticoids and some anticonvulsants can also lead to loss of bone density and fractures. Subjects who exhibit these risk factors frequently are found to have osteoporosis or a pre-osteoporosis condition when assessed by BMD. Also at risk for developing osteoporosis or osteopenia are subjects who lead inactive lifestyles or who have been subjected to extended bed rest, subjects who engage in smoking, or excessive consumption of alcohol. Several risk rules and indices have been constructed integrating these variables into clinically useful measurements of absolute or relative risk, such as the Osteoporosis Risk Assessment Instrument (ORAI), the Osteoporosis Self-Assessment Tool (OST), among others; such multi-variate approaches tend to have reasonably high sensitivity for osteoporosis, but low specificity. For example, the OST has been reported to identify over 90 percent of women with osteoporosis (and 100% of those over 65), but more than half of the women identified by this tool as requiring BMD resting were found on test to actually not have osteoporosis (See Chapter 10, Bone Health and Osteoporosis: A Report of the Surgeon General (2004) and also Woolf & Pfleger, Burden of Major Musculoskeletal Conditions, Bulletin of the World Health Organization (2003) 81: 646-656).

A substantial detection gap remains for those who are at risk for bone fractures, yet are as yet asymptomatic or remain undiagnosed by BMD, who may or may not yet exhibit conventional risk factors, or are currently deemed clinically to be at low risk and have not yet been diagnosed with osteoporosis or pre-osteoporosis. Furthermore, there is a substantial gap in risk stratification of those with conventional risk factors, which commonly lack specificity, and a detection gap for earlier diagnosis of high risk for future osteoporosis or pre-osteoporosis, when therapeutic intervention or lifestyle modification may have the greatest effect in maintaining bone health. The biomarkers and methods of the present invention allow one of skill in the art to identify, diagnose, or otherwise assess those subjects who do not exhibit any symptoms of osteoporosis or pre-osteoporosis, but who nonetheless may be at risk for developing or experiencing bone fracture or diminished bone mass.

The term biomarker (also known in the art as “biological marker”) can refer to measurable and quantifiable biological parameters (e.g., specific enzyme concentration, specific hormone concentration, specific gene phenotype distribution in a population, presence of biological substances) which serve as indices for health- and physiology-related assessments, such as disease risk, psychiatric disorders, environmental exposure and its effects, disease diagnosis, metabolic processes, substance abuse, pregnancy, cell line development, epidemiologic studies, etc. A biomarker can also be a characteristic that is objectively measured and evaluated as an indicator of normal biological processes, pathogenic processes, or pharmacologic responses to a therapeutic intervention. A biomarker may be measured on a biosample from a subject (such as a blood, urine, or tissue test), it may be a recording obtained from a person (such as a bone mineral density test), or it may be an imaging test (for example, quantitative ultrasound, CT scan, or bone absorptiometry).

Biomarkers can indicate a variety of health or disease characteristics, including the level or type of exposure to an environmental factor, genetic susceptibility, genetic responses to exposures, markers of subclinical or clinical disease, or indicators of response to therapy. Thus, biomarkers can be used as indicators of disease trait (risk factor or risk marker), disease state (preclinical or clinical), or disease rate (progression). Accordingly, biomarkers can be classified as antecedent biomarkers (identifying the risk of developing an illness), screening biomarkers (screening for subclinical disease), diagnostic biomarkers (recognizing overt disease), staging biomarkers (categorizing disease severity), or prognostic biomarkers (predicting future disease course, including recurrence and response to therapy, and monitoring efficacy of therapy).

The term “biomarker” in the context of the present invention encompasses, without limitation, proteins, nucleic acids, polymorphisms of proteins and nucleic acids, elements (such as calcium), metabolites, and other analytes. Biomarkers can also include mutated proteins or mutated nucleic acids. The term “analyte” as used herein can mean any substance to be measured and can encompass electrolytes and elements, such as calcium. Finally, biomarkers can also refer to non-analyte physiological markers of health status encompasses other clinical characteristics, without limitation, such as age, bone density or bone mineral density (BMD), gender, menopause, body size, body mass index (BMI), smoking status, past usage of certain medications (such as glucocorticosteroids), family history of fracture, and ethnicity. One hundred and ninety-one biomarkers have been identified as being present in subjects who have osteoporosis or osteopenia.

Proteins and nucleic acids whose expression levels are changed in subjects who have osteoporosis, osteopenia, pre-osteoporosis or bone fractures or are predisposed to developing same are summarized in Table 1 and are collectively referred to herein as “bone metabolism-associated proteins”, “OSTEORISKMARKER polypeptides”, or “OSTEORISKMARKER proteins”. The corresponding nucleic acids encoding the polypeptides are referred to as “bone metabolism risk-associated nucleic acids”, “bone metabolism risk-associated genes”, “OSTEORISKMARKER nucleic acids”, or “OSTEORISKMARKER genes”. Unless indicated otherwise, “OSTEORISKMARKER”, “bone metabolism risk-associated proteins”, “bone metabolism risk-associated nucleic acids” are meant to refer to any of the sequences disclosed herein. Metabolites of the OSTEORISKMARKER proteins or nucleic acids can also be measured, herein referred to as “OSTEORISKMARKER metabolites”. Non-analyte physiological markers of health status (e.g., age, gender, bone density, bone mass, and other non-analyte measurements commonly used as conventional risk factors) are referred to as “OSTEORISKMARKER physiology”. Calculated indices created from mathematically combining measurements of one or more of the aforementioned classes of OSTEORISKMARKERS are referred to as “OSTEORISKMARKER indices”. Proteins, nucleic acids, polymorphisms, mutated proteins and mutated nucleic acids, metabolites, and other analytes are, as well as common physiological measurements and indices constructed from any of the preceding entities, are included in the broad category of “OSTEORISKMARKERS”.

The methods disclosed herein are used with subjects at risk for developing bone fractures, osteoporosis, osteopenia, or pre-osteoporosis, subjects who have already been diagnosed with a bone fracture, osteoporosis, osteopenia or pre-osteoporosis, subjects undergoing treatment and/or therapies for osteoporosis, osteopenia or pre-osteoporosis. The methods of the present invention can also be used to monitor or select a treatment regimen for a subject who has osteoporosis, osteopenia or pre-osteoporosis, and to screen subjects who have not been previously diagnosed as having osteoporosis, osteopenia or pre-osteoporosis, such as subjects who exhibit risk factors for osteoporosis, osteopenia or pre-osteoporosis, or to assess a subject's future risk of developing osteoporosis, pre-osteoporosis, bone fracture, osteopenia or diminished bone mass. Preferably, the methods of the present invention are used to identify and/or diagnose subjects who are asymptomatic for osteoporosis, pre-osteoporosis, or osteopenia. “Asymptomatic” means not currently exhibiting the traditional symptoms, including but not limited to diminished bone mass, decreased bone calcification, and bone fragility.

The methods of the present invention may also be used to identify and/or diagnose subjects at higher risk of osteoporosis, osteopenia or pre-osteoporosis based solely on single measurements of conventional risk factors.

### Diagnostic and Prognostic Methods

The risk of developing osteoporosis, osteopenia or pre-osteoporosis can be detected by examining an effective amount of OSTEORISKMARKER proteins, nucleic acids, polymorphisms, metabolites, and other analytes in a test sample (i.e., a subject derived sample). Subjects identified as having an increased risk of osteoporosis, pre-osteoporosis, or osteopenia can optionally be selected to receive treatment regimens, such as administration of prophylactic or therapeutic compounds, or implementation of exercise regimens or dietary supplements to prevent or delay the onset of osteoporosis or osteopenia. A sample isolated from the subject can comprise, for example, blood, plasma, blood cells, serum, bone marrow, ascites fluid, interstitial fluid (such as, but not limited to, gingival crevicular fluid), urine, sputum, cerebrospinal fluid, saliva, or other bodily fluids.

The amount of the OSTEORISKMARKER protein, nucleic acid, polymorphism, metabolite, or other analyte can be measured in a test sample and compared to the normal control level. The term “normal control level”, means the level of an OSTEORISKMARKER protein, nucleic acid, polymorphism, metabolite, or other analyte typically found in a subject not suffering from osteoporosis and not likely to have a osteoporotic or pre-osteoporotic condition, i.e., relative to samples collected from young subjects who were monitored until advanced age and were found not to develop osteoporosis or osteopenia. Alternatively, the normal control level can mean the level of an OSTEORISKMARKER protein, nucleic acid, polymorphism, metabolite, or other analyte typically found in a subject suffering from osteoporosis or osteopenia. The normal control level can be a range or an index. Alternatively, the normal control level can be a database of patterns from previously tested subjects. A change in the level in the subject-derived sample of an OSTEORISKMARKER protein, nucleic acid, polymorphism, metabolite, or other analyte compared to the normal control level can indicate that the subject is suffering from or is at risk of developing osteoporosis or osteopenia. In contrast, when the methods are applied prophylactically, a similar level compared to the normal control level in the subject-derived sample of an OSTEORISKMARKER protein, nucleic acid, polymorphism, metabolite, or other analyte can indicate that the subject is not suffering from or is not at risk or at low risk of developing bone fractures, osteoporosis or pre-osteoporosis.

The difference in the level of OSTEORISKMARKERS is statistically significant. By “statistically significant”, it is meant that the alteration is greater than what might be expected to happen by chance alone. Statistical significance can be determined by method known in the art. For example statistical significance can be determined by p-value. The p-value is a measure of probability that a difference between groups during an experiment happened by chance. (P(z>zobserved)). For example, a p-value of 0.01 means that there is a 1 in 100 chance the result occurred by chance. The lower the p-value, the more likely it is that the difference between groups was caused by treatment. An alteration is statistically significant if the p-value is at least 0.10. Preferably, the p-value is 0.05, 0.04, 0.03, 0.02, 0.01, 0.005, 0.001 or less.

The “diagnostic accuracy” of a test, assay, or method concerns the ability of the test, assay, or method to distinguish between subjects having osteoporosis or at risk for osteoporosis is based on whether the subjects have a “clinically significant presence” of an OSTEORISKMARKER. By “clinically significant presence”, it is meant that the presence of the OSTEORISKMARKER (i.e., mass, such as milligrams, nanograms, or mass per volume, such as milligrams per deciliter or copy number of a transcript per unit volume) in the subject (typically in a sample from the subject) is higher than the predetermined cut-off point (or threshold value) for that OSTEORISKMARKER and therefore indicates that the subject has osteoporosis for which the sufficiently high presence of that protein, nucleic acid, polymorphism, metabolite or analyte is a marker.

The terms “high degree of diagnostic accuracy” and “very high degree of diagnostic accuracy” refer to the test or assay for that OSTEORISKMARKER with the predetermined cut-off point correctly (accurately) indicating the presence or absence of the disease or pre-disease condition. A perfect test would have perfect accuracy. Thus, for subjects who have the condition, the test would indicate only positive test results and would not report any of those subjects as being “negative” (there would be no “false negatives”). In other words, the “sensitivity” of the test (the true positive rate, or detection of disease when disease is truly present) would be 100%. On the other hand, for subjects who did not have osteoporosis, the test would indicate only negative test results and would not report any of those subjects as being “positive” (there would be no “false positives”). In other words, the “specificity” (the true negative rate, or the recognition of absence of disease when disease is truly absent) would be 100%. See, i.e., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, i.e., a clinical diagnostic test.

Reference values or limits can be generated with the use of cross-sectional analyses of a reference sample (usually a healthy sample derived from a subject free of the disease of interest), and an arbitrary percentile cutpoint (typically the 95th or 97.5^{th} percentile) is chosen to define abnormality. The reference range is the interval between the minimum and the maximum reference values. At least 200 individuals are required within each category for the formulation of reference limits for subgroups (eg, defined by age and sex). Cutpoints that define abnormality are typically the lower and the upper bounds of the 95% reference interval (between the lower 2.5th percentile and upper 97.5th percentile), but they may vary on the basis of the intent. The reference interval may be moved up or down according to the tradeoff between the implications (medical, ethical, social, psychological, and economic) of false-negative and false-positive results, i.e., the consequences of missing disease, the availability and efficacy of treatment for people with abnormal values, and the costs associated with follow-up of abnormal results.

Several issues must be considered when reference values or limits are interpreted. First, a select proportion of “normal” individuals typically exceed the reference limits on the basis of the percentile chosen. Second, values that lie within statistically defined reference limits may not indicate health in a given individual, especially when the person comes from a group inherently different from the one used to derive the reference values. Third, a change in values within the reference range may indicate pathology. Accordingly, delta limits have been formulated to evaluate the change in biomarker values within an individual (in response to disease or therapy) relative to the physiological intraindividual fluctuation of values. Fourth, a value within the reference range may not necessarily be desirable, especially when the prevalence of undesirable values of a biomarker in the population is high. For example, bone mineral density tests are known to generate values that differ markedly among individuals in a defined group, and have been known to generate disparate results among different bones of the same individual.

Discrimination limits can also used to indicate abnormal biomarker values. Such limits can be generated by evaluating the degree of overlap between patients with and without disease in cross-sectional studies. Discrimination limits trigger decisions (they are referred to as decision thresholds). The discrimination thresholds can be varied depending on the relative importance of missing disease versus that of misclassifying nondiseased individuals.

A third method is to define “undesirable” biomarker levels by relating values to the incidence of disease and seeking a threshold beyond which risk escalates. For most osteoporosis and osteopenic risk factors, there is a continuous gradient of risk across the range of risk factors, and a majority of individuals in a population could be classified as having undesirable levels. “Treatment” levels (especially for pharmacological treatment) of risk factors may therefore differ from undesirable levels, being defined by the risk factor thresholds for which there is good evidence (typically from large randomized controlled trials) that treatment for values above a limit does more benefit than harm. Often such treatment levels may be defined not only by the level of the specific risk factor being evaluated but by taking into consideration absolute risk of disease based on the values of several other risk factors. For other biomarkers, the choice of the optimal cutpoint defining abnormality remains to be described and may vary with the purpose. Once abnormal thresholds of markers are formulated, biomarker performance can be assessed with the use of computed indices and risk prediction algorithms as defined herein.

Changing the cut point or threshold value of a test (or assay) usually changes the sensitivity and specificity, but in a qualitatively inverse relationship. For example, if the cut point is lowered, more subjects in the population tested will typically have test results over the cut point or threshold value. Because subjects who have test results above the cut point are reported as having the disease, condition, or syndrome for which the test is being run, lowering the cut point will cause more subjects to be reported as having positive results (i.e., that they have osteoporosis or pre-osteoporosis). Thus, a higher proportion of those who have osteoporosis will be indicated by the test to have it. Accordingly, the sensitivity (true positive rate) of the test will be increased. However, at the same time, there will be more false positives because more people who do not have the disease, condition, or syndrome (i.e., people who are truly “negative”) will be indicated by the test to have OSTEORISKMARKER values above the cut point and therefore to be reported as positive (i.e., to have the disease, condition, or syndrome) rather than being correctly indicated by the test to be negative. Accordingly, the specificity (true negative rate) of the test will be decreased. Similarly, raising the cut point will tend to decrease the sensitivity and increase the specificity. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points.

There is, however, an indicator that allows representation of the sensitivity and specificity of a test, assay, or method over the entire range of cut points with just a single value. That indicator is derived from a Receiver Operating Characteristics (“ROC”) curve for the test, assay, or method in question. See, i.e., Shultz, “Clinical Interpretation Of Laboratory Procedures,” chapter 14 in Teitz, Fundamentals of Clinical Chemistry, Burtis and Ashwood (eds.), 4^{th} edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronory Artery Disease,” Clin. Chem., 1992, 38(8): 1425-1428.

An ROC curve is an x-y plot of sensitivity on the y-axis, on a scale of zero to one (i.e., 100%), against a value equal to one minus specificity on the x-axis, on a scale of zero to one (i.e., 100%). In other words, it is a plot of the true positive rate against the false positive rate for that test, assay, or method. To construct the ROC curve for the test, assay, or method in question, subjects can be assessed using a perfectly accurate or “gold standard” method that is independent of the test, assay, or method in question to determine whether the subjects are truly positive or negative for the disease, condition, or syndrome (for example, bone mineral density scanning is a gold standard test for diagnosis of osteoporosis, as coronary angiography is a gold standard test for the presence of coronary atherosclerosis). The subjects can also be tested using the test, assay, or method in question, and for varying cut points, the subjects are reported as being positive or negative according to the test, assay, or method. The sensitivity (true positive rate) and the value equal to one minus the specificity (which value equals the false positive rate) are determined for each cut point, and each pair of x-y values is plotted as a single point on the x-y diagram. The “curve” connecting those points is the ROC curve. Each point on the ROC curve indicates the conditional probability of a positive test result from a random diseased individual exceeding that from a random non-diseased person. Likelihood ratios (LR) are calculated with the use of sensitivity and specificity data and are helpful in determining the likelihood of obtaining a positive test result in someone with disease compared with someone without disease (LR+), and the likelihood of getting a negative result in someone with disease compared with someone without disease (LR−).

The area under the curve (“AUC”) is the indicator that allows representation of the sensitivity and specificity of a test, assay, or method over the entire range of cut points with just a single value. The maximum AUC is one (a perfect test) and the minimum area is one half, which would denote no discrimination between disease and non-disease groups. The closer the AUC is to one, the better is the accuracy of the test.

Appropriate use of biomarker results requires integrating pretest probabilities with biomarker test results (expressed as sensitivity/specificity or as LR) to estimate the post-test probability of disease. Predictive values use this concept to facilitate interpretation of test results, taking into consideration disease prevalence. Even for a test with high sensitivity and specificity, false positive tests will outnumber true-positive tests when disease prevalence is very low, and false-negative tests will outnumber true-negative tests when disease prevalence is very high.

Biomarkers (whether for screening, diagnosis, or prognosis) are also evaluated in terms of their discrimination and calibration capabilities. Discrimination refers to the ability of the biomarker (by itself or as part of a composite score) to distinguish “case” from “noncase” in cross-sectional studies or to differentiate “those who will develop disease” from “those who will not” in longitudinal investigations. Typically, the c-statistic (or concordance index) is used as the metric of model discrimination and is equivalent to the area under the ROC curve. The c-statistic is the probability that in 2 randomly paired individuals (one with and one without disease), a given test correctly identifies the one with disease. It is important to note that the c-statistic is a metric of overall performance. It is possible for 2 tests to have the same c-statistic, yet one biomarker may be superior to the other in terms of performance at select thresholds.

Calibration is an indicator of the ability of a biomarker (or a model incorporating the biomarker) to predict risk relates to the actual observed risk in subgroups of the population. The Hosmer-Lemeshow goodness-of-fit statistic is often used as an indicator of model calibration. For this purpose, the sample is divided into deciles of risk, and the observed number of events is compared with the expected number of events. Thus, risk prediction algorithms have been developed that incorporate select biomarkers and enable clinicians to predict the absolute event rates of disease; examples include estimating the risk of osteoporosis or pre-osteoporosis, given values of risk factors, assessing the risk of bone fracture and/or diminished bone mass in subjects not previously diagnosed as having osteoporosis or pre-osteoporosis, and appraising the risk of bone fracture in subjects with established osteoporosis or osteopenia. Models can be recalibrated if they uniformly underestimate or overestimate risk.

By a “high degree of diagnostic accuracy”, it is meant a test or assay (such as the test of the invention for determining the clinically significant presence of OSTEORISKMARKERS, which thereby indicates the presence of osteoporosis or osteopenia) in which the AUC (area under the ROC curve for the test or assay) is at least 0.70, desirably at least 0.75, more desirably at least 0.80, preferably at least 0.85, more preferably at least 0.90, and most preferably at least 0.95.

By a “very high degree of diagnostic accuracy”, it is meant a test or assay in which the AUC (area under the ROC curve for the test or assay) is at least 0.875, desirably at least 0.90, more desirably at least 0.925, preferably at least 0.95, more preferably at least 0.975, and most preferably at least 0.98.

The predictive value of any test depends on the sensitivity and specificity of the test, and on the prevalence of the condition in the population being tested. This notion, based on Bayes' theorem, provides that the greater the likelihood that the condition being screened for is present in an individual or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative. Furthermore, under such differing settings, and additionally under differing disease acuities, appropriate and acceptable standards and requirements of test performance may also vary.

“Risk” in the context of the present invention can mean “absolute” risk, which refers to that percentage change that an event will occur over a specific time period. “Relative” risk refers to the ratio or odds of a subject's risk compared either to low risk or average risk, which can vary by how clinical risk factors are assessed. Subjects suffering from or at risk of developing osteoporosis or osteopenia can be diagnosed or identified by methods known in the art. Such methods include, but are not limited to, bone biopsy, bone mineral density test (BMD), single photon absorptiometry (SPA), dual photon absorptiometry (DPA), dual-energy X-ray absorptiometry (DEXA or DXA), quantitative computed tomography QCT), and quantitative ultrasound (QUS).

Risk prediction for bone health and diseases can also encompass risk prediction algorithms and computed indices that assess and estimate a subject's absolute or relative risk for developing osteoporosis or osteopenia. Mathematical models incorporating assessment of osteoporosis and pre-osteoporosis risk factors have been used to predict general levels of risk (e.g., low, intermediate, or high) and to estimate the yearly percentage risk (absolute risk) or future events. Estimates or scores derived from these models are commonly referred to in the art as “global” risk scores. Risk assessment using such predictive mathematical algorithms and computed indices has increasingly been incorporated into guidelines for diagnostic testing and treatment and encompass indices obtained from, inter alia, multi-stage, stratified samples from a representative population. Examples of such tools for the global assessment of osteoporosis and bone fracture risk include the National Osteoporosis checklist, the Osteoporosis Risk Assessment Instrument (ORAI), the Simple Calculated Osteoporosis Risk Estimation (SCORE), the Osteoporosis Self-assessment Tool (OST), the calculated score from the Dubbo Osteoporosis Epidemiology Study, and the FRACTURE Index score, developed and validated in the Study of Osteoporotic Fractures (SOF), among others.

Despite the numerous studies and algorithms that have been used to assess the risk of osteoporosis or osteopenia, the evidence-based, multiple risk factor assessment approach is only moderately accurate for the prediction of short- and long-term risk of manifesting bone fracture, diminished bone mass, or bone fragility, in asymptomatic or otherwise healthy subjects (See Chapter 8, Bone Health and Osteoporosis: A Report of the Surgeon General (2004) for a summary of such scores and their performance). The OSTEORISKMARKERS and methods of use disclosed herein provides a tool that can be used in combination with such risk prediction algorithms to assess, identify, or diagnose subjects who are asymptomatic and do not exhibit the conventional risk factors.

The data derived from risk prediction algorithms and from the methods of the present invention can be analyzed by linear regression. Linear regression analysis models the relationship between two variables by fitting a linear equation to observed data. One variable is considered to be an explanatory variable, and the other is considered to be a dependent variable. For example, given a population of subjects, algorithms discussed herein can be an explanatory variable and analyzed against levels of one or more OSTEORISKMARKERS within the same subjects, and OSTEORISKMARKER indices developed which achieve the best fit to the risk prediction algorithms.

A linear regression line has an equation of the form Y=a+bX, where X is the explanatory variable and Y is the dependent variable. The slope of the line is b, and a is the intercept (the value of y when x=0). A numerical measure of association between two variables is the “correlation coefficient,” which is a value between −1 and 1 indicating the strength of the association of the observed data for the two variables. The most common method for fitting a regression line is the method of least-squares. This method calculates the best-fitting line for the observed data by minimizing the sum of the squares of the vertical deviations from each data point to the line (if a point lies on the fitted line exactly, then its vertical deviation is 0). Because the deviations are first squared, then summed, there are no cancellations between positive and negative values.

After a regression line has been computed for a group of data, a point which lies far from the line (and thus has a large residual value) is known as an outlier. Such points may represent erroneous data, or may indicate a poorly fitting regression line. If a point lies far from the other data in the horizontal direction, it is known as an influential observation. The reason for this distinction is that these points have may have a significant impact on the slope of the regression line. Once a regression model has been fit to a group of data, examination of the residuals (the deviations from the fitted line to the observed values) allows one of skill in the art to investigate the validity of the assumption that a linear relationship exists. Plotting the residuals on the y-axis against the explanatory variable on the x-axis reveals any possible non-linear relationship among the variables, or might alert the skilled artisan to investigate “lurking variables.” A “lurking variable” exists when the relationship between two variables is significantly affected by the presence of a third variable which has not been included in the modeling effort.

Linear regression analyses can be used, inter alia, to predict the risk of developing osteoporosis or pre-osteoporosis based upon correlating the levels of OSTEORISKMARKERS in a sample from a subject in combination with, for example, validated osteoporosis risk prediction algorithms as discussed herein, or other known methods of diagnosing or predicting the prevalence of disease, as in those developed elsewhere (for example, in the assessment of atherosclerotic risk). Of particular use, however, are non-linear equations and analyses, such as logarithmic regression, to determine the relationship between known predictive models of bone disease and levels of OSTEORISKMARKERS detected in a subject sample.

Where actual longitudinal long term subject outcomes, such as the conversion rate to osteoporosis or osteopenia, are also known in a population, several additional techniques can used in developing classification algorithms to distinguish those who will develop osteoporosis or bone fractures from those who will not. Results from the OSTEORISKMARKER indices thus derived can then be validated through their calibration with actual results, that is, by comparing the predicted versus observed rate of disease in a given population, and the best predictive OSTEORISKMARKERS selected for and optimized through mathematical models of increased complexity. Beyond the simple non-linear transformations, such as logarithmic regression, of particular interest in this use of the present invention are structural and synactic classification algorithms, and methods of risk index construction, utilizing pattern recognition features, including established techniques such as the Kth-Nearest Neighbor, Boosting, Decision Trees, Neural Networks, Bayesian Networks, Support Vector Machines, and Hidden Markov Models.

Hierarchical clustering can be performed in the derivation of a predictive model, where the Pearson correlation is employed as the clustering metric. One approach is to consider a patient osteoporosis or pre-osteoporosis dataset as a “learning sample” in a problem of “supervised learning”. CART is a standard in applications to medicine (Singer (1999) Recursive Partitioning in the Health Sciences, Springer), which may be modified by transforming any qualitative features to quantitative features; sorting them by attained significance levels, evaluated by sample reuse methods for Hotelling's T2 statistic; and suitable application of the lasso method. Problems in prediction are turned into problems in regression without losing sight of prediction, indeed by making suitable use of the Gini criterion for classification in evaluating the quality of regressions.

This approach has led to what is termed FlexTree (Huang (2004) PNAS 101:10529-10534). FlexTree has performed very well in simulations and when applied to SNP and other forms of data. Software automating FlexTree has been developed. Alternatively, LARTree or LART may be used (Turnbull (2005) Classification Trees with Subset Analysis Selection by the Lasso, Stanford University). The name reflects binary trees, as in CART and FlexTree; the lasso, as has been noted; and the implementation of the lasso through what is termed LARS by Efron et al. (2004) Annals of Statistics 32:407-451. See, also, Huang et al. (2004) Tree-structured supervised learning and the genetics of hypertension. Proc Natl Acad Sci USA. 101(29): 10529-34.

Other methods of analysis that may be used include logic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as PAM software and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome.

To provide significance ordering, the false discovery rate (FDR) may be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (see Tusher et al. (2001) PNAS 98, 5116-21, herein incorporated by reference). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value may be applied to the correlations between experimental profiles.

Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pairwise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation. In an alternative analytical approach, variables chosen in the cross-sectional analysis are separately employed as predictors. Given the specific outcome, the random lengths of time each patient will be observed, and selection of proteomic and other features, a parametric approach to analyzing survival may be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and functions of them are available with this model.

Furthermore the application of such techniques to panels of multiple OSTEORISKMARKERS is provided, as is the use of such combination to create single numerical “risk indices” or “risk scores” encompassing information from multiple OSTEORISKMARKER inputs. Individual OSTEORISKMARKERS may also be included or excluded in the panel of OSTEORISKMARKERS used in the calculation of the OSTEORISKMARKER indices so derived above, based on various measures of relative performance and calibration in validation, and employing through repetitive training methods such as forward, reverse, and stepwise selection, as well as with genetic algorithm approaches, with or without the use of constraints on the complexity of the resulting OSTEORISKMARKER indices.

The above measurements of diagnostic accuracy for OSTEORISKMARKERS are only a few of the possible measurements of the clinical performance of the invention. It should be noted that the appropriateness of one measurement of clinical accuracy or another will vary based upon the clinical application, the population tested, and the clinical consequences of any potential misclassification of subjects. Other important aspects of the clinical and overall performance of the invention include the selection of OSTEORISKMARKERS so as to reduce overall OSTEORISKMARKER variability (whether due to method (analytical) or biological (pre-analytical variability, for example, as in diurnal variation), or to the integration and analysis of results (post-analytical variability) into indices and cut-off ranges), to assess analyte stability or sample integrity, or to allow the use of differing sample matrices amongst blood, serum, plasma, urine, etc.

Levels of an effective amount of one or more OSTEORISKMARKERS also allows for the course of treatment of osteoporosis or pre-osteoporosis to be monitored. In this method, a biological sample can be provided from a subject undergoing treatment regimens, e.g., hormonal treatment, for osteoporosis or osteopenia. Such treatment regimens can include, but are not limited to, exercise regimens, dietary supplementation of calcium, and treatment with therapeutics or prophylactics used in subjects diagnosed or identified with osteoporosis. If desired, biological samples are obtained from the subject at various time points before, during, or after treatment. Levels of an effective amount of one or more OSTEORISKMARKER(S) can then be determined and compared to a reference value, e.g., a control subject or population whose osteoporosis state is known or an index value or baseline value. The reference sample or index value or baseline value may be taken or derived from one or more subjects who have been exposed to the treatment. Alternatively, the reference sample or index value or baseline value may be taken or derived from one or more subjects who have not been exposed to the treatment. For example, samples may be collected from subjects who have received initial treatment for osteoporosis or osteopenia and subsequent treatment for osteoporosis or osteopenia to monitor the progress of the treatment. A reference value can also comprise a value derived from risk prediction algorithms or computed indices from population studies such as those disclosed herein.

Differences in the genetic makeup of subjects can result in differences in their relative abilities to metabolize various drugs, which may increase bone mineral content. Subjects that have osteoporosis, osteopenia, or pre-osteporosis, or at risk for developing bone fracture, osteoporosis, pre-osteoporosis, or osteopenia can vary in age, body or bone mass index (BMI), and, in female subjects, whether they are pre- or post-menopausal. Accordingly, the OSTEORISKMARKERS disclosed herein allow for a putative therapeutic or prophylactic to be tested from a selected subject in order to determine if the agent is a suitable for treating or preventing osteoporosis, pre-osteoporosis, or osteopenia in the subject.

To identify therapeutics or drugs that are appropriate for a specific subject, a test sample from the subject can be exposed to a therapeutic agent or a drug, and the level of one or more of OSTEORISKMARKERS can be determined. Examples of such therapeutics or drugs frequently used in osteoporosis or osteopenia treatments, and may modulate bone mineral content include, but are not limited to, bisphosphonates such as alendronate, risedronate, etidronate, pamidronate, clodronate, and ibandronate, selective estrogen-receptor modulators (SERMs) such as raloxifene, tamoxifen, and toremifine, anabolic therapies such as teriparatide and strontium ranelate, and recombinant peptide fragments of parathyroid hormone, estrogen/progesterone replacement therapies, monoclonal antibodies, inhibitors of receptor activator of nuclear factor KB ligand (RANKL), inhibitors of cathepsin K, antagonists of integrin Avβ3 (also known in the art as vitronectin), calcitonin, and dietary supplements such as calcium and vitamin D. Such therapeutics or drugs have been prescribed for subjects diagnosed with osteoporosis or osteopenia, and may modulate bone mineral content.

A subject sample can be incubated in the presence of a candidate agent and the pattern of the levels of one or more OSTEORISKMARKER(S) in the test sample is measured and compared to a reference profile, i.e., a pre-osteoporosis reference molecular profile or an non-pre-osteoporosis reference molecular profile or an index value or baseline value. The test agent can be any compound or composition. For example, the test agents are agents frequently used in osteoporosis, pre-osteoporosis, or osteopenia treatment regimens and are described herein.

Accordingly, the present invention provides a method for treating one or more subjects at risk for developing osteoporosis, pre-osteoporosis, osteopenia or bone fracture, comprising: detecting the presence of increased levels of at least two different OSTEORISKMARKERS present in a sample from the one or more subjects; and treating the one or more subjects with one or more bone mineral content-modulating drugs until altered levels of the at least two different OSTEORISKMARKERS return to a baseline value measured in one or more subjects at low risk for developing osteoporosis, pre-osteoporosis, osteopenia, or bone fracture.

Also provided by the present invention is a method for treating one or more subjects having osteoporosis, pre-osteoporosis, or osteopenia comprising: detecting the presence of increased levels of at least two different OSTEORISKMARKERS present in a sample from the one or more subjects; and treating the one or more subjects with one or more bone mineral content-modulating drugs until altered levels of the at least two different OSTEORISKMARKERS return to a baseline value measured in one or more subjects at low risk for developing bone fracture, osteoporosis, pre-osteoporosis, or osteopenia.

Comparison can be performed on test and reference samples measured concurrently or at temporally distinct times. An example of the latter is the use of compiled expression or molecular quantity information, i.e., a sequence database, which assembles information about expression levels or molecular quantities of OSTEORISKMARKERS.

If the reference sample, i.e., a control sample, is from a subject that does not have osteoporosis or osteopenia, or if the reference sample reflects a value that is relative to a person that has a high likelihood of rapid progression to osteoporosis, pre-osteoporosis, or osteopenia, a similarity in the amount of the OSTEORISKMARKER analytes in the test sample and the reference sample indicates that the treatment is efficacious. However, a change in the amount of the OSTEORISKMARKER in the test sample and the reference sample indicates a less favorable clinical outcome or prognosis.

By “efficacious”, it is meant that the treatment leads to a decrease in the amount of one or more OSTEORISKMARKERS, an increase in bone mineral density or bone quality as measured by a bone mineral density test or bone biopsy, or a decrease in the risk of fracture in a subject. Assessment of the risk of fracture and increases or decreases in bone mineral density can be achieved using standard clinical protocols. Efficacy can be determined in association with any known method for diagnosing, identifying, or treating osteoporosis, pre-osteoporosis or osteopenia.

The subject is preferably a mammal. The mammal can be a human, non-human primate, mouse, rat, dog, cat, horse, or cow, but are not limited to these examples. Mammals other than humans can be advantageously used as subjects as animal models of osteoporosis and osteopenia. A subject can be male or female. A subject can be one who has been previously diagnosed or identified as having osteoporosis, pre-osteoporosis or osteopenia, and optionally has already undergone treatment for osteoporosis, pre-osteoporosis or osteopenia. Alternatively, a subject can also be one who has not been previously diagnosed as having osteoporosis, pre-osteoporosis or osteopenia.

A subject can also be one who is suffering from or at risk of developing osteoporosis, pre-osteoporosis or osteopenia. Subjects suffering from or at risk of developing osteoporosis, pre-osteoporosis or osteopenia can be diagnosed or identified by methods known in the art. For example, osteoporosis is frequently diagnosed by measuring the bone mineral content in a bone mineral density test. Bone biopsy may be useful in unusual forms of osteoporosis, such as osteoporosis in young adults. Biopsy provides information about the rate of bone turnover and the presence of secondary forms of osteoporosis, such as myeloma and systemic mastocytosis.

A bone mineral density test measures how many grams of calcium and other bone minerals are packed into a segment of bone. The amount of bone mineral is referred to as “bone mineral content”. The higher the mineral content, the denser the bones are, and the denser the bones are, the stronger they are and are thus less likely to break. Bone mineral density tests are typically performed on bones that are most likely to break due to osteoporosis, such as the lumbar vertebrae, the femur, and the bones of the wrist and forearm. Other peripheral bones can also be measured, such as the bones of the fingers and heel. Bone mineral density is determined by measuring the amount of bone mineral (calcium hydroxyapatite) per unit volume of bone tissue. X-rays or gamma rays are often used to quantify bone mineral density. In quantitative terms, bone mineral density is the amount of calcium hydroxyapatite, or Ca₁₀(PO₄)₆(OH)₂ per unit volume of bone tissue examined.

Imaging modalities used in bone mineral density tests include single photon absorptiometry (SPA), where a single energy photon beam is passed through bone and soft tissue to a detector. The amount of mineral in the path is then be quantified. The amount of soft tissue the beam penetrate need to be small so the distal radius is usually utilized. Dual photon absorptiometry (DPA) uses a photon beam that has two distinct energy peaks. One energy peak will be more absorbed by soft tissue and the other by bone. The soft tissue component then can be mathematically subtracted and the bone mineral density is determined. Dual-energy X-ray absorptiometry (DEXA; DXA) uses an X-ray source instead of an isotope. This technique is superior because the radiation source does not decay and the energy stays constant over time. Scan times are much shorter than with DPA and radiation dose is very low. DEXA can be used as an accurate and precise method to monitor changes in bone density in subjects undergoing treatments. Other methods include quantitative computed tomography (QCT), wherein measurement of bone mineral density can be achieved by standard CT scanners with software packages that allow them to determine bone density in the hip or spine. This technique provides for true three-dimensional imaging and reports bone mineral density as true volume density measurements. The advantage of QCT is its ability to isolate the area of interest from surrounding tissues. Also frequently used is quantitative ultrasound (QUS), which uses high-frequency sound waves to measure bone mineral density and assess bone microarchitecture, a measure of bone quality. QUS requires placement between a transponder and a receiver, and is limited to testing of distal skeletal sites.

The results of a bone mineral density test are reported in two numbers: T-scores and Z-scores. A T-score is the bone density compared with what is normally expected in a healthy young adult subject. The T-score is the number of units that the bone density is above or below a standard. According to the WHO definitions, T-scores above −1 often indicate subjects having normal bone density. T-scores ranging between −1 and −2.5 classify subjects as having osteopenia, wherein bone density is below normal and which may lead to osteoporosis. T-scores below −2.5 classify subjects as having osteoporosis. The Z-score is the number of standard deviations above or below what is normally expected for a person of the subject's age, sex, weight, and ethnic or racial origin. Z-scores less than −1.5 may indicate that factors other than aging is the cause of bone loss.

According to the invention, several techniques can be used to construct OSTEORISKMARKER panels which use some or all of the 191 OSTEORISKMARKERS disclosed herein, which may be combined with concurrent measurement of conventional risk factors and methods of assessment for osteoporosis, osteopenia or pre-osteoporosis. These OSTEORISKMARKER selection techniques may exploit input from one or more sources: from actual OSTEORISKMARKER data derived from their measurement in similar populations, from specific selected OSTEORISKMARKER characteristics (such as molecular class, association with physiological functions, cellular or extracellular localization, and resulting kinetics of expression across disease states and progression), and from molecular pathway and related interaction network analysis of the OSTEORISKMARKERS.

As mentioned above, in one embodiment of the invention, the OSTEORISKMARKER composition and mathematical algorithms used in individual OSTEORISKMARKER panels and indices are developed through the use of classification algorithms which are derived from actual measurements and longitudinal outcomes (such as whether or not the subject subsequently developed osteoporosis or osteopenia from a pre-osteoporosis baseline starting condition) or existing validated risk index algorithms, taken over many subjects in a population similar to that which will subsequently be tested by the OSTEORISKMARKER invention.

Also according to the invention, OSTEORISKMARKERS can be selected into panels that comprise biomarkers specific to a particular disease (based on physiological pathways, molecular pathways or other protein interaction networks), disease site, disease stage, disease kinetics, and can also comprise markers that can be used to exclude and distinguish osteoporosis, pre-osteoporosis and related diseases from each other (“exclusion markers”). Such panels can comprise one or more OSTEORISKMARKERS, but can also comprise one OSTEORISKMARKER, where that one OSTEORISKMARKER can provide information about several pathways, diseases, disease kinetics, or disease stages. Such panels can comprise additional OSTEORISKMARKERS other than the 191 representative OSTEORISKMARKERS disclosed in Table 1.

Table 1 comprises 191 representative OSTEORISKMARKERS of the present invention. One skilled in the art will recognize that the OSTEORISKMARKERS presented herein encompasses all forms and variants, including but not limited to, polymorphisms, isoforms, mutants, derivatives, precursors including nucleic acids, receptors (including soluble and transmembrane receptors), ligands, and post-translationally modified variants, as well as any multi-unit nucleic acid, protein, and glycoprotein structures comprised of any of the OSTEORISKMARKERS as constituent subunits of the fully assembled structure. Furthermore, common degradation products of the OSTEORISKMARKERS shown below are also encompassed. By way of example and without limitation, several forms of collagen (e.g. collagen type I (COL1A1 and COL1A2; the most abundant human collagen), collagen type II (COL2A1 articular cartilage associated), collagen type III (COL3A1, granulation, arterial and fibroblast associated), collagen type IX (COL9A1, COL9A2, COL9A3), collagen type X (COL10A1, hypertrophic and mineralizing collagen), collagen type XIV (COL14A1), amongst the other approximately 28 known types of collagen) are hereby claimed, as are their component genes, variants, mRNA transcripts, monomeric peptide chains (alpha-1 and alpha-2 for collagen type I), procollagens, procollagen carboxyterminal (e.g. PICP) and aminoterminal (e.g. PINP) propetides, tropocollagen, collagen fibrils, collagen fibers, crosslinked fibrillar collagens, their crosslinked carboxyterminal and aminoterminal telopeptides (e.g. CTX and NTX), and the degradation and resorption byproducts such as the hydroxypyridinium crosslinks of collagen (PYD and DPD), are herein expressly claimed, regardless of whether these individual forms are specifically noted in any figure or table herein. One skilled in the art will furthermore recognize that multiple other precursor, degradation and other products of derived from collagen are present, including individual enantiomeric forms, and that the presence and concentration relationships of several of the individual related collagen products are individually useful (e.g. the ratio of the non-isomerized α-L octapeptide of CTX (α-CTX) to the β-L isomerized isoaspartyl perptide of CTX (β-CTX) is known to be elevated in the urine of patients with untreated Paget's disease of bone).

TABLE 1
OSTEORISKMARKERS

| OSTEORISKMARKER | Official Name | Common Name | Symbol |
|---|---|---|---|
| 1 | acid phosphatase 5, tartrate | Acid phosphatase Tartrate- | ACP5 |
| | resistant | resistant, Type 5b | |
| | | (osteoclasts), TRAP, tartrate | |
| | | resistant acid phosphatase 5, | |
| | | TRACP 5b (produced in | |
| | | osteoclasts) and TRACP 5a | |
| | | (produced in other cells) | |
| 2 | advanced glycosylation | RAGE, advanced | AGER |
| | end product-specific | glycosylation end product- | |
| | receptor | specific receptor RAGE3; | |
| | | advanced glycosylation end | |
| | | product-specific receptor | |
| | | variant sRAGE1; advanced | |
| | | glycosylation end product- | |
| | | specific receptor variant | |
| | | sRAGE2; receptor for | |
| | | advanced glycosylation end- | |
| | | products; soluble receptor | |
| 3 | alpha-2-HS-glycoprotein | A2HS, AHS, FETUA, | AHSG |
| | | HSGA, Alpha-2HS- | |
| | | glycoprotein; fetuin-A | |
| 4 | arachidonate 15- | arachidonate 15-lipoxygenase | ALOX15 |
| | lipoxygenase | | |
| 5 | alkaline phosphatase, | alkaline phosphatase, | ALPL |
| | liver/bone/kidney | liver/bone/kidney, AP-TNAP, | |
| | | HOPS, TNAP, TNSALP, | |
| | | alkaline | |
| | | phosphomonoesterase; | |
| | | glycerophosphatase; tissue | |
| | | non-specific alkaline | |
| | | phosphatase; tissue- | |
| | | nonspecific ALP | |
| 6 | anthrax toxin receptor 2 | capillary morphogenesis | ANTXR2 |
| | | gene-2 (CMG-2), CMG-2, | |
| | | CMG2, ISH, JHF, capillary | |
| | | morphogenesis protein 2 | |
| 7 | apolipoprotein E | APO E, AD2, apoprotein, | APOE |
| | | Alzheimer disease 2 | |
| | | (APOE*E4-associated, late | |
| | | onset); apolipoprotein E | |
| | | precursor; apolipoprotein E3 | |
| 8 | androgen receptor | androgen receptor; | AR |
| | (dihydrotestosterone | dihydrotestosterone receptor, | |
| | receptor; testicular | AIS, DHTR, HUMARA, KD, | |
| | feminization; spinal and | NR3C4, SBMA, SMAX1, | |
| | bulbar muscular atrophy; | TFM, androgen receptor; | |
| | Kennedy disease) | dihydrotestosterone receptor | |
| 9 | amphiregulin | AR, CRDGF, SDGF, | AREG |
| | (schwannoma-derived | amphiregulin; colorectum | |
| | growth factor) | cell-derived growth factor; | |
| | | schwannoma-derived growth | |
| | | factor | |
| 10 | ATPase, Ca++ | ATPase, Ca++ transporting, | ATP2B3 |
| | transporting, plasma | plasma membrane 3, | |
| | membrane 3 | PMCA3, plasma membrane | |
| | | calcium ATPase 3; plasma | |
| | | membrane calcium pump | |
| | | isoform 3 | |
| 11 | Best5 protein (Rat) | Rat | Best5 |
| 12 | bone gamma- | Osteocalcin, BGP, PMF1, | BGLAP |
| | carboxyglutamate (gla) | gamma-carboxyglutamic | |
| | protein (osteocalcin) | acid-containing protein; | |
| | | osteocalcin; polyamine- | |
| | | modulated factor 1 | |
| 13 | biglycan | DSPG1, PG-S1, PGI, | BGN |
| | | SLRR1A, bone/cartilage | |
| | | proteoglycan-I; dermatan | |
| | | sulphate proteoglycan I; small | |
| | | leucine-rich protein 1A | |
| 14 | bone morphogenetic | BMP2A | BMP2 |
| | protein 2 | | |
| 15 | bone morphogenetic | VGR, VGR1, Vg-related | BMP6 |
| | protein 6 | sequence; transforming | |
| | | growth factor-beta; vegetal | |
| | | related growth factor (TGFB- | |
| | | related); vegetal-related | |
| | | (TGFB related) cytokine | |
| 16 | calcitonin/calcitonin- | Calcitonin, CALC1, CGRP, | CALCA |
| | related polypeptide, alpha | CGRP-I, CGRP1, CT, KC, | |
| | | calcitonin; katacalcin | |
| 17 | calcitonin receptor | calcitonin receptor, CRT, | CALCR |
| | | CTR, CTR1 | |
| 18 | calreticulin | RO, SSA, cC1qR, Sicca | CALR |
| | | syndrome antigen A | |
| | | (autoantigen Ro; calreticulin); | |
| | | autoantigen Ro | |
| 19 | capping protein (actin | capping protein (actin | CAPG |
| | filament), gelsolin-like | filament), AFCP, MCP, actin- | |
| | | regulatory protein CAP-G; | |
| | | gelsolin-like capping protein; | |
| | | macrophage capping protein | |
| 20 | calcium-sensing receptor | FHH, FIH, GPRC2A, HHC, | CASR |
| | (hypocalciuric | HHC1, NSHPT, PCAR1, | |
| | hypercalcemia 1, severe | calcium sensing receptor; | |
| | neonatal | calcium-sensing receptor; | |
| | hyperparathyroidism) | extracellular calcium-sensing | |
| | | receptor; parathyroid Ca(2+)- | |
| | | sensing receptor 1 | |
| 21 | chemokine (C-C motif) | macrophage activating | CCL18 |
| | ligand 18 (pulmonary and | protein, Gc - AMAC-1, | |
| | activation-regulated) | AMAC1, CKb7, DC-CK1, | |
| | | DCCK1, MIP-4, PARC, | |
| | | SCYA18, CC chemokine | |
| | | ligand 18; alternative | |
| | | macrophage activation- | |
| | | associated CC chemokine 1; | |
| | | chemokine (C-C), dendritic; | |
| | | dendritic cell chemokine 1; | |
| | | macrophage inflammatory | |
| | | protein 4; pulmonary and | |
| | | activation-regulated | |
| | | chemokine; small inducible | |
| | | cytokine A18; small inducible | |
| | | cytokine subfamily A (Cys- | |
| | | Cys), member 18; small | |
| | | inducible cytokine subfamily | |
| | | A (Cys-Cys), member 18, | |
| | | pulmonary and activation- | |
| | | regulated | |
| 22 | chemokine (C-C motif) | CC-CKR-3, CD193, CKR3, | CCR3 |
| | receptor 3 | CMKBR3, CC chemokine | |
| | | receptor 3; b-chemokine | |
| | | receptor; eosinophil CC | |
| | | chemokine receptor 3; | |
| | | eosinophil eotaxin receptor | |
| 23 | CD200 receptor 1 | CD200R, HCRTR2, | CD200R1 |
| | | MOX2R, OX2R, MOX2 | |
| | | receptor; cell surface | |
| | | glycoprotein OX2 receptor; | |
| | | cell surface glycoprotein | |
| | | receptor CD200 | |
| 24 | CD44 molecule (Indian | CD44, CDW44, ECMR-III, | CD44 |
| | blood group) | IN, LHR, MC56, MDU2, | |
| | | MDU3, MIC4, MUTCH-I, | |
| | | Pgp1, CD44 antigen; CD44 | |
| | | antigen (Indian blood group); | |
| | | CD44 antigen (homing | |
| | | function and Indian blood | |
| | | group system); CD44 | |
| | | epithelial domain (CD44E); | |
| | | CDW44 antigen; GP90 | |
| | | lymphocyte homing/adhesion | |
| | | receptor; Hermes antigen; | |
| | | antigen gp90 homing | |
| | | receptor; cell adhesion | |
| | | molecule (CD44); cell surface | |
| | | glycoprotein CD44; | |
| | | extracellular matrix receptor- | |
| | | III; heparan sulfate | |
| | | proteoglycan; hyaluronate | |
| | | receptor; phagocytic | |
| | | glycoprotein I | |
| 25 | cyclin-dependent kinase | cyclin dependent kinase | CDKN1C |
| | inhibitor 1C (p57, Kip2) | inhibitor 1c, BWCR, BWS, | |
| | | KIP2, WBS, p57, Beckwith- | |
| | | Wiedemann syndrome; | |
| | | cyclin-dependent kinase | |
| | | inhibitor 1C | |
| 26 | chitinase 3-like 1 (cartilage | GP39, HC-gp39, HCGP-3P, | CHI3L1 |
| | glycoprotein-39) | YKL40, YYL-40, cartilage | |
| | | glycoprotein-39; chitinase 3- | |
| | | like 1 | |
| 27 | chordin-like 1 | Ventropin, CHL, NRLN1, | CHRDL1 |
| | | VOPT, chordin-like; chordin- | |
| | | like 1 variant; neuralin 1 | |
| 28 | chordin-like 2 | BNF1, CHL2, FKSG37, | CHRDL2 |
| | | breast tumor novel factor 1 | |
| 29 | chloride channel 7 | CLC-7, CLC7, OPTA2 | CLCN7 |
| 30 | cannabinoid receptor 1 | cannabinoid receptor 1, | CNR1 |
| | (brain) | CANN6, CB-R, CB1, CB1A, | |
| | | CB1K5, CNR, central | |
| | | cannabinoid receptor | |
| 31 | cannabinoid receptor 2 | cannabinoid receptor 2 | CNR2 |
| | (macrophage) | (macrophage), CB2, CX5 | |
| 32 | ciliary neurotrophic factor | CNTFR alpha; ciliary | CNTFR |
| | receptor | neurotrophic factor receptor | |
| | | alpha precursor | |
| 33 | collagen, type X, alpha | collagen X, alpha-1 | COL10A1 |
| | 1(Schmid metaphyseal | polypeptide; collagen, type X, | |
| | chondrodysplasia) | alpha 1; collagen, type X, | |
| | | alpha 1 (Schmid metaphyseal | |
| | | chondrodysplasia) | |
| 34 | collagen, type I, alpha 1 | collagen α-1; Collagen I, | COL1A1 |
| | | alpha-1 polypeptide; | |
| | | Collagen alpha 1 chain; alpha | |
| | | 1 type I collagen; collagen | |
| | | alpha 1 chain type I; collagen | |
| | | of skin, tendon and bone, | |
| | | alpha-1 chain; osteogenesis | |
| | | imperfecta type IV; pro- | |
| | | alpha-1 collagen type 1; type | |
| | | I collagen alpha 1 chain; type | |
| | | I collagen pro alpha 1(I) | |
| | | chain propeptide; type II | |
| | | procollagen gene fragment | |
| 35 | collagen, type II, alpha 1 | AOM, COL11A3, SEDC, | COL2A1 |
| | (primary osteoarthritis, | alpha 1 type II collagen; | |
| | spondyloepiphyseal | alpha-1 collagen type II; | |
| | dysplasia, congenital) | arthroophthalmopathy, | |
| | | progressive; cartilage | |
| | | collagen; chondrocalcin, | |
| | | included; collagen II, alpha-1 | |
| | | polypeptide; collagen alpha 1 | |
| | | type II | |
| 36 | carboxypeptidase B2 | thrombin activatable | CPB2 |
| | (plasma, carboxypeptidase | fibrinolysis inhibitor (TAFI), | |
| | U) | CPU, PCPB, TAFI, | |
| | | carboxypeptidase B-like | |
| | | protein; carboxypeptidase U; | |
| | | plasma carboxypeptidase B2; | |
| | | thrombin-activable | |
| | | fibrinolysis inhibitor; | |
| | | thrombin-activatable | |
| | | fibrinolysis inhibitor | |
| 37 | C-reactive protein, | C-Reactive Protein, CRP, | CRP |
| | pentraxin-related | PTX1; DNA Marker: CRP | |
| | | gene +1444C > T variant | |
| 38 | colony stimulating factor 1 | M-CSF, colony stimulating | CSF1 |
| | (macrophage) | factor 1; macrophage colony | |
| | | stimulating factor | |
| 39 | catenin (cadherin- | β-catenin, CTNNB, catenin | CTNNB1 |
| | associated protein), beta 1, | (cadherin-associated protein), | |
| | 88 kDa | beta 1 (88 kD); catenin | |
| | | (cadherin-associated protein), | |
| | | beta 1 (88 kDa | |
| 40 | cathepsin K | CTS02, CTSO, CTSO1, | CTSK |
| | (pycnodysostosis) | CTSO2, PKND, PYCD, | |
| | | cathepsin K; cathepsin O1; | |
| | | cathepsin O2; cathepsin X | |
| 41 | cathepsin L | CATL, MEP, major excreted | CTSL |
| | | protein | |
| 42 | cytochrome P450, family | CPT7, CYP17, P450C17, | CYP17A1 |
| | 17, subfamily A, | S17AH, cytochrome P450, | |
| | polypeptide 1 | family 17; cytochrome P450, | |
| | | subfamily XVII (steroid 17- | |
| | | alpha-hydroxylase), adrenal | |
| | | hyperplasia; cytochrome | |
| | | p450 XVIIA1; steroid 17- | |
| | | alpha-hydroxylase/17,20 | |
| | | lyase; steroid 17-alpha- | |
| | | monooxygenase | |
| 43 | cytochrome P450, family | ARO, ARO1, CPV1, CYAR, | CYP19A1 |
| | 19, subfamily A, | CYP19, P-450AROM, | |
| | polypeptide 1 | aromatase; cytochrome P450, | |
| | | family 19; cytochrome P450, | |
| | | subfamily XIX | |
| | | (aromatization of androgens); | |
| | | estrogen synthetase; | |
| | | flavoprotein-linked | |
| | | monooxygenase; microsomal | |
| | | monooxygenase | |
| 44 | cytochrome P450, family | AHH, AHRR, CP11, CYP1, | CYP1A1 |
| | 1, subfamily A, | P1-450, P450-C, P450DX, | |
| | polypeptide 1 | P450 form 6; aryl | |
| | | hydrocarbon hydroxylase; | |
| | | cytochrome P1-450, dioxin- | |
| | | inducible; cytochrome P450 | |
| | | 1A1 variant; cytochrome | |
| | | P450, subfamily I (aromatic | |
| | | compound-inducible), | |
| | | polypeptide 1; flavoprotein- | |
| | | linked monooxygenase; | |
| | | microsomal monooxygenase; | |
| | | xenobiotic monooxygenase | |
| 45 | cytochrome P450, family | 1,25-@dihydroxyvitamin D3 | CYP24A1 |
| | 24, subfamily A, | 24-hydroxylase; 24-ohase; | |
| | polypeptide 1 | cytochrome P450, family 24; | |
| | | cytochrome P450, subfamily | |
| | | XXIV (vitamin D 24- | |
| | | hydroxylase); exo- | |
| | | mitochondrial protein; | |
| | | vitamin D 24-hydroxylase | |
| 46 | cytochrome P450, family | CYP27C1, CP27, CTX, | CYP27A1 |
| | 27, subfamily A, | CYP27, 5-beta-cholestane-3- | |
| | polypeptide 1 | alpha, 7-alpha, 12-alpha-triol | |
| | | 26-hydroxylase; 5-beta- | |
| | | cholestane-3-alpha, 7-alpha, | |
| | | 12-alpha-triol 27- | |
| | | hydroxylase; cholestanetriol | |
| | | 26-monooxygenase; | |
| | | cytochrome P-450C27/25; | |
| | | cytochrome P450, subfamily | |
| | | XXVIIA (steroid 27- | |
| | | hydroxylase, | |
| | | cerebrotendinous | |
| | | xanthomatosis), polypeptide | |
| | | 1; sterol 27-hydroxylase; | |
| | | vitamin D(3) 25-hydroxylase | |
| 47 | cytochrome P450, family | CP2B, CYP1, CYP1alpha, | CYP27B1 |
| | 27, subfamily B, | CYP27B, P450c1, PDDR, | |
| | polypeptide 1 | VDD1, VDDR, VDDRI, | |
| | | VDR, 25 hydroxyvitamin D3- | |
| | | 1-alpha hydroxylase; 25- | |
| | | OHD-1 alpha-hydroxylase; | |
| | | 25-hydroxyvitamin D-1- | |
| | | alpha-hydroxylase; P450C1- | |
| | | alpha; P450VD1-alpha; VD3 | |
| | | 1A hydroxylase; VDDR I; | |
| | | calcidiol 1-monooxygenase; | |
| | | cytochrome P450, subfamily | |
| | | XXVIIB (25-hydroxyvitamin | |
| | | D-1-alpha-hydroxylase), | |
| | | polypeptide 1; cytochrome | |
| | | P450, subfamily XXVIIB, | |
| | | polypeptide 1 | |
| 48 | dickkopf homolog 1 | DKK-1, SK, dickkopf | DKK1 |
| | (Xenopus laevis) | (Xenopus laevis) homolog 1; | |
| | | dickkopf homolog 1; | |
| | | dickkopf related protein-1; | |
| | | dickkopf-1; dickkopf-1 like | |
| 49 | endothelin 3 | endothelin III: ET3, ET3, | EDN3 |
| | | truncated endothelin 3 | |
| 50 | engrailed homolog 1 | engrailed homolog 1 | EN1 |
| 51 | estrogen receptor 1 | ER, ESR, ESRA, Era, | ESR1 |
| | | NR3A1, (estrogen receptor | |
| | | 1); estrogen receptor 1 | |
| | | (alpha); oestrogen receptor; | |
| | | steroid hormone receptor | |
| 52 | estrogen receptor 2 (ER | ER-BETA, ESR-BETA, | ESR2 |
| | beta) | ESRB, ESTRB, Erb, NR3A2, | |
| | | estrogen receptor beta | |
| 53 | exostoses (multiple) 1 | EXT, ttv, exostosin 1 | EXT1 |
| 54 | exostoses (multiple) 2 | ext2 exostosin 2 - SOTV | EXT2 |
| 55 | fetuin B | fetuin-mineral complex, | FETUB |
| | | 16G2, Gugu, IRL685, fetuin- | |
| | | like protein | |
| 56 | fibroblast growth factor 2 | Fibrin, BFGF, FGFB, | FGF2 |
| | (basic) | HBGH-2, basic fibroblast | |
| | | growth factor; basic fibroblast | |
| | | growth factor bFGF; | |
| | | fibroblast growth factor 2; | |
| | | heparin-binding growth factor | |
| | | 2 precusor; prostatropin | |
| 57 | fibroblast growth factor 23 | Phosphatonin, ADHR, | FGF23 |
| | | HPDR2, HYPF, PHPTC, | |
| | | tumor-derived | |
| | | hypophosphatemia inducing | |
| | | factor | |
| 58 | FOS-like antigen 1 | FRA1, fra-1, FOS-like | FOSL1 |
| | | antigen-1 | |
| 59 | frizzled-related protein | FRE, FRITZ, FRP-3, FRZB- | FRZB |
| | | 1, FRZB-PEN, FRZB1, | |
| | | FZRB, SFRP3, SRFP3, hFIZ, | |
| | | frizzled (Drosophila) | |
| | | homolog-related | |
| 60 | frizzled homolog 10 | Frizzled homolog 10, FZ-10, | FZD10 |
| | (Drosophila) | FzE7, hFz10, frizzled | |
| | | (Drosophila) homolog 10; | |
| | | frizzled 10; frizzled 10 | |
| | | precursor | |
| 61 | group-specific component | DBP, DBP/GC, VDBG, | GC |
| | (vitamin D binding | VDBP, vitamin D binding | |
| | protein) | protein; vitamin D-binding | |
| | | alpha-globulin; vitamin D- | |
| | | binding protein; vitamin D- | |
| | | binding protein/group | |
| | | specific component | |
| 62 | growth differentiation | Myostatin, MSTN | GDF8 |
| | factor 8 | | |
| 63 | growth hormone 1 | growth hormone, GH, GH-N, | GH1 |
| | | GHN, hGH-N, pituitary | |
| | | growth hormone | |
| 64 | G protein-coupled receptor | G Protein Coupled Receptor | GPR109A |
| | 109A | HM74a; HM74a, HM74b, | |
| | | PUMAG, Puma-g, G protein- | |
| | | coupled receptor HM74a | |
| 65 | major histocompatibility | HLA A, Class I HLA-B- | HLA-A |
| | complex, class I, A | 3201; HLA class I; HLA | |
| | | class I antigen; HLA class I | |
| | | heavy chain; HLA class I | |
| | | molecule; MHC class 1 | |
| | | antigen; MHC class I; MHC | |
| | | class I HLA-A; MHC class I | |
| | | HLA-A antigen; MHC class I | |
| | | antigen; MHC class I antigen | |
| | | HLA-A; MHC class I antigen | |
| | | HLA-A heavy chain; MHC | |
| | | class I antigen HLA-A2407; | |
| | | MHC class I antigen heavy | |
| | | chain; MHC class I antigen | |
| | | precusor; MHC leukocyte | |
| | | antigen; alpha 2 domain; | |
| | | alpha 1 domain; antigen | |
| | | presenting molecule; heavy | |
| | | chain of HLA-A antigen; | |
| | | histocompatibility molecule; | |
| | | leucocyte antigen; leucocyte | |
| | | antigen A; leucocyte antigen | |
| | | A alpha chain; leucocyte | |
| | | antigen B; leucocyte antigen | |
| | | class I; leukocyte antigen; | |
| | | leukocyte antigen class I; | |
| | | leukocyte antigen class I-A; | |
| | | leukocyte antigen, HLA-A2 | |
| | | variant; leukocyte antigen- | |
| | | A*0104N; lymphocyte | |
| | | antigen | |
| 66 | haptoglobin | Haptoglobin; hp2-alpha | HP |
| 67 | heat shock 70 kDa protein | BIP, GRP78, MIF2, Heat- | HSPA5 |
| | 5 (glucose-regulated | shock 70 kD protein-5 | |
| | protein, 78 kDa) | (glucose-regulated protein, | |
| | | 78 kD); heat shock 70 kD | |
| | | protein 5 (glucose-regulated | |
| | | protein, 78 kD) | |
| 68 | islet amyloid polypeptide | Amylin, DAP, IAP, Islet | IAPP |
| | | amyloid polypeptide | |
| | | (diabetes-associated peptide; | |
| | | amylin) | |
| 69 | integrin-binding | BNSP, BSP, BSP-II, SP-II, | IBSP |
| | sialoprotein (bone | Integrin-binding sialoprotein | |
| | sialoprotein, bone | (bone sialoprotein II); bone | |
| | sialoprotein II) | sialoprotein II; bone | |
| | | sialoprotein; integrin-binding | |
| | | sialoprotein | |
| 70 | insulin-like growth factor 1 | IGF-1; somatomedin C; | IGF1 |
| | (somatomedin C) | insulin-like growth factor-1 | |
| 71 | insulin-like growth factor 2 | IGF-II polymorphisms | IGF2 |
| | (somatomedin A) | (somatomedin A); C11orf43, | |
| | | INSIGF, pp9974, insulin-like | |
| | | growth factor 2; insulin-like | |
| | | growth factor II; insulin-like | |
| | | growth factor type 2; putative | |
| | | insulin-like growth factor II | |
| | | associated protein | |
| 72 | insulin-like growth factor | insulin-like growth factor | IGFBP1 |
| | binding protein 1 | binding protein-1 (IGFBP-1); | |
| | | AFBP, IBP1, IGF-BP25, | |
| | | PP12, hIGFBP-1, IGF- | |
| | | binding protein 1; alpha- | |
| | | pregnancy-associated | |
| | | endometrial globulin; | |
| | | amniotic fluid binding | |
| | | protein; binding protein-25; | |
| | | binding protein-26; binding | |
| | | protein-28; growth hormone | |
| | | independent-binding protein; | |
| | | placental protein 12 | |
| 73 | interleukin 10 | IL-10, CSIF, IL-10, IL10A, | IL10 |
| | | TGIF, cytokine synthesis | |
| | | inhibitory factor | |
| 74 | interleukin 1, alpha | IL 1; IL-1A, IL1, IL1- | IL1A |
| | | ALPHA, IL1F1, IL1A | |
| | | (IL1F1); hematopoietin-1; | |
| | | preinterleukin 1 alpha; pro- | |
| | | interleukin-1-alpha | |
| 75 | interleukin 1, beta | interleukin-1 beta (IL-1 beta); | IL1B |
| | | IL-1, IL1-BETA, IL1F2, | |
| | | catabolin; preinterleukin 1 | |
| | | beta; pro-interleukin-1-beta- | |
| | | IL-1B(+3954)T (associated | |
| | | with higher CRP levels) | |
| 76 | interleukin 1 receptor | interleukin-1 receptor | IL1RN |
| | antagonist | antagonist (IL-1Ra); ICIL- | |
| | | 1RA, IL-1ra3, IL1F3, IL1RA, | |
| | | IRAP, IL1RN (IL1F3); | |
| | | intracellular IL-1 receptor | |
| | | antagonist type II; | |
| | | intracellular interleukin-1 | |
| | | receptor antagonist (icIL- | |
| | | 1ra); type II interleukin-1 | |
| | | receptor antagonist - DNA | |
| | | Marker - DNA Marker: IL- | |
| | | 1RN(VNTR)*2 (associated | |
| | | with lower CRP levels) | |
| 77 | interleukin 2 | interleukin-2 (IL-2); IL-2, | IL2 |
| | | TCGF, lymphokine, T cell | |
| | | growth factor; aldesleukin; | |
| | | interleukin-2; involved in | |
| | | regulation of T-cell clonal | |
| | | expansion | |
| 78 | interleukin 2 receptor, beta | IL-2R, CD122, P70-75, | IL2RB |
| | | CD122 antigen; high affinity | |
| | | IL-2 receptor beta subunit; | |
| | | interleukin 2 receptor beta | |
| 79 | interleukin 4 | BSF1, IL-4, B-cell | IL4 |
| | | stimulatory factor 1; | |
| | | lymphocyte stimulatory | |
| | | factor 1 | |
| 80 | interleukin 6 (interferon, | Interleukin-6 (IL-6), BSF2, | IL6 |
| | beta 2) | HGF, HSF, IFNB2, IL-6 | |
| 81 | interleukin 6 receptor | interleukin-6 receptor, soluble | IL6R |
| | | (sIL-6R); CD126, IL-6R-1, | |
| | | IL-6R-alpha, IL6RA, CD126 | |
| | | antigen; interleukin 6 receptor | |
| | | alpha subunit | |
| 82 | interleukin 8 | Interleukin-8 (IL-8), 3-10C, | IL8 |
| | | AMCF-I, CXCL8, GCP-1, | |
| | | GCP1, IL-8, K60, LECT, | |
| | | LUCT, LYNAP, MDNCF, | |
| | | MONAP, NAF, NAP-1, | |
| | | NAP1, SCYB8, TSG-1, b- | |
| | | ENAP, CXC chemokine | |
| | | ligand 8; LUCT/interleukin- | |
| | | 8; T cell chemotactic factor; | |
| | | beta-thromboglobulin-like | |
| | | protein; chemokine (C—X—C | |
| | | motif) ligand 8; emoctakin; | |
| | | granulocyte chemotactic | |
| | | protein 1; lymphocyte- | |
| | | derived neutrophil-activating | |
| | | factor; monocyte derived | |
| | | neutrophil-activating protein; | |
| | | monocyte-derived neutrophil | |
| | | chemotactic factor; | |
| | | neutrophil-activating factor; | |
| | | neutrophil-activating peptide | |
| | | 1; neutrophil-activating | |
| | | protein 1; protein 3-10C; | |
| | | small inducible cytokine | |
| | | subfamily B, member 8 | |
| 83 | inhibin, alpha | inhibin, alpha; A-inhibin | INHA |
| | | subunit precursor; inhibin | |
| | | alpha subunit | |
| 84 | inhibin, beta B (activin AB | Inhibin, beta-2; activin AB | INHBB |
| | beta polypeptide) | beta polypeptide precursor; | |
| | | inhibin beta B subunit | |
| 85 | integrin, beta 3 (platelet | glycoprotein Iib/IIIa; CD61, | ITGB3 |
| | glycoprotein IIIa, antigen | GP3A, GPIIIa, integrin beta | |
| | CD61) | chain, beta 3; platelet | |
| | | glycoprotein IIIa precursor- | |
| | | DNA Marker; platelet | |
| | | glycoprotein IIIa Leu33Pro | |
| | | allele/Pl(A1/A2) | |
| | | polymorphism of GPIIIa/ | |
| | | Pl(A2) (Leu33Pro) | |
| | | polymorphism of beta(3) | |
| | | integrins/polymorphism | |
| | | responsible for the Pl(A2) | |
| | | alloantigen on the beta(3)- | |
| | | component | |
| 86 | KISS1 receptor | G-protein coupled receptor | KISS1R |
| | | 54; AXOR12, GPR54, G | |
| | | protein-coupled receptor 54; | |
| | | metastin receptor | |
| 87 | klotho | klotho | KL |
| 88 | leptin (obesity homolog, | Leptin; OB, OBS, leptin; | LEP |
| | mouse) | leptin (murine obesity | |
| | | homolog); obesity; obesity | |
| | | (murine homolog, leptin) | |
| 89 | leptin receptor | leptin receptor, soluble; | LEPR |
| | | CD295, OBR, OB receptor | |
| 90 | leucine-rich repeat- | G protein-coupled receptor | LGR4 |
| | containing G protein- | 48; GPR48 | |
| | coupled receptor 4 | | |
| 91 | leukemia inhibitory factor | CDF, D-FACTOR, HILDA, | LIF |
| | (cholinergic differentiation | cholinergic differentiation | |
| | factor) | factor | |
| 92 | low density lipoprotein | BMND1, EVR1, HBM, LR3, | LRP5 |
| | receptor-related protein 5 | LRP7, OPPG, OPS, VBCH2, | |
| | | low density lipoprotein | |
| | | receptor-related protein 7; | |
| | | osteoporosis pseudoglioma | |
| | | syndrome | |
| 93 | low density lipoprotein | low density lipoprotein- | LRP6 |
| | receptor-related protein 6 | related protein 6 | |
| 94 | latent transforming growth | transforming growth factor | LTBP3 |
| | factor beta binding protein 3 | (TGF)-beta binding protein 3 | |
| 95 | matrix Gla protein | GIG36, MGLAP, NTI, | MGP |
| | | Gamma-carboxyglutamic | |
| | | acid protein, matrix; Matrix | |
| | | gamma-carboxyglutamic acid | |
| | | protein; Matrix gamma- | |
| | | carboxylglutamate protein | |
| 96 | matrix metallopeptidase 2 | Matrix Metalloproteinases | MMP2 |
| | (gelatinase A, 72 kDa | (MMP), MMP-2, CLG4, | |
| | gelatinase, 72 kDa type IV | CLG4A, MMP-II, MONA, | |
| | collagenase) | TBE-1, 72 kD type IV | |
| | | collagenase; collagenase type | |
| | | IV-A; matrix | |
| | | metalloproteinase 2; matrix | |
| | | metalloproteinase 2 | |
| | | (gelatinase A, 72 kD | |
| | | gelatinase, 72 kD type IV | |
| | | collagenase); matrix | |
| | | metalloproteinase 2 | |
| | | (gelatinase A, 72 kDa | |
| | | gelatinase, 72 kDa type IV | |
| | | collagenase); matrix | |
| | | metalloproteinase-II; | |
| | | neutrophil gelatinase | |
| 97 | MAS-related GPR, | human rta-like g protein- | MRGPRF |
| | member F | coupled receptor; mas related | |
| | | gene F, GPR140, GPR168, | |
| | | RTA, mrgF, G protein- | |
| | | coupled receptor 168; G | |
| | | protein-coupled receptor | |
| | | MrgF; seven transmembrane | |
| | | helix receptor | |
| 98 | 5,10- | methylenetetrahydrofolate | MTHFR |
| | methylenetetrahydrofolate | reductase; | |
| | reductase (NADPH) | methylenetetrahydrofolate | |
| | | reductase intermediate form, | |
| | | red blood cell 5- | |
| | | methyltetrahydrofolate (RBC | |
| | | 5-MTHFR); (MTHFR | |
| | | A1298C) mutation | |
| 99 | myosin, light polypeptide | myosin light chain II, cardiac; | MYL2 |
| | 2, regulatory, cardiac, slow | CMH10, MLC2, myosin light | |
| | | chain 2 | |
| 100 | type 2a sodium-phosphate | type 2a sodium-phosphate | NaKTrans2a |
| | cotransporter | cotransporter | |
| 101 | neurofibromin 1 | neurofibromin 1; NFNS, | NF1 |
| | (neurofibromatosis, von | VRNF, WSS, Neurofibromin | |
| | Recklinghausen disease, | (neurofibromatosis, type I); | |
| | Watson disease | neurofibromin | |
| 102 | natriuretic peptide | B-type Natriuretic Peptide | NPPB |
| | precursor B | (BNP), BNP, brain type | |
| | | natriuretic peptide, pro- | |
| | | BNP?, NPPB | |
| 103 | neuropeptide Y | neuropeptide Y; PYY4 | NPY |
| 104 | neuropeptide Y receptor | G Protein-Coupled Receptor | NPY1R |
| | Y1 | NPY1; NPYR, modulator of | |
| | | neuropeptide Y receptor | |
| 105 | nuclear receptor subfamily | Glucocorticoid receptor; | NR3C1 |
| | 3, group C, member 1 | GCCR, GCR, GR, GRL, | |
| | | glucocorticoid receptor; | |
| | | nuclear receptor subfamily 3, | |
| | | group C, member 1 | |
| 106 | osteoclast-associated | PIGR3, osteoclast associated | OSCAR |
| | receptor | receptor OSCAR-S1; | |
| | | osteoclast associated receptor | |
| | | OSCAR-S2; polymeric | |
| | | immunoglobulin receptor 3 | |
| | | precursor | |
| 107 | osteopetrosis associated | GIPN, GL, HSPC019, GAIP- | OSTM1 |
| | transmembrane protein 1 | interacting protein N | |
| | | terminus; grey-lethal | |
| | | osteopetrosis | |
| 108 | oxoglutarate (alpha- | human P2Y-like GPCR | OXGR1 |
| | ketoglutarate) receptor 1 | protein (G protein-coupled | |
| | | receptor 80; G protein- | |
| | | coupled receptor 99; P2Y-like | |
| | | nucleotide receptor; seven | |
| | | transmembrane helix | |
| | | receptor) | |
| 109 | oxytocin, prepro- | Oxytocin, OT, OT-NPI, | OXT |
| | (neurophysin I) | oxytocin-neurophysin I; | |
| | | oxytocin-neurophysin I, | |
| | | preproprotein | |
| 110 | RF(Arg-Phe)amide family | 26RFa, QRFP, P518 | P518 |
| | 26 amino acid peptide | precursor protein; control of | |
| | | feeding behavior; | |
| | | neuropeptide | |
| 111 | pregnancy-associated | Pregnancy-associated plasma | PAPPA |
| | plasma protein A, | protein a; ASBABP2, | |
| | pappalysin 1 | DIPLA1, IGFBP-4ase, | |
| | | PAPA, PAPP-A, PAPPA1, | |
| | | aspecific BCL2 ARE-binding | |
| | | protein 2; differentially | |
| | | placenta 1 expressed protein; | |
| | | insulin-like growth factor- | |
| | | dependent IGF binding | |
| | | protein-4 protease; pregnacy- | |
| | | associated plasma protein A; | |
| | | pregnancy-associated plasma | |
| | | protein A | |
| 112 | phosphodiesterase 4B, | phosphodiesterase 4B; | PDE4B |
| | cAMP-specific | DPDE4, PDEIVB, cAMP- | |
| | (phosphodiesterase E4 | specific 3′,5′-cyclic | |
| | dunce homolog, | phosphodiesterase 4B; dunce- | |
| | Drosophila) | like phosphodiesterase E4; | |
| | | phosphodiesterase 4B, | |
| | | cAMP-specific; | |
| | | phosphodiesterase 4B, | |
| | | cAMP-specific (dunce | |
| | | (Drosophila)-homolog | |
| | | phosphodiesterase E4) | |
| 113 | phosphodiesterase 4D, | phosphodiesterase 4D; | PDE4D |
| | cAMP-specific | DPDE3, HSPDE4D, | |
| | (phosphodiesterase E3 | PDE4DN2, STRK1, cAMP- | |
| | dunce homolog, | specific phosphodiesterase | |
| | Drosophila) | 4D; cAMP-specific | |
| | | phosphodiesterase PDE4D6; | |
| | | dunce-like phosphodiesterase | |
| | | E3; phosphodiesterase 4D, | |
| | | cAMP-specific (dunce | |
| | | (Drosophila)-homolog | |
| | | phosphodiesterase E3) | |
| 114 | PDZ and LIM domain 4 | RIL, LIM domain protein; | PDLIM4 |
| | | enigma homolog | |
| 115 | peptidase D | X-pro dipeptidase; | PEPD |
| | | PROLIDASE, Xaa-Pro | |
| | | dipeptidase; proline | |
| | | dipeptidase | |
| 116 | phosphate regulating | phosphate regulating | PHEX |
| | endopeptidase homolog, | endopeptidase homolog; | |
| | X-linked | HPDR, HPDR1, HYP, | |
| | (hypophosphatemia, | HYP1, PEX, XLH, X-linked | |
| | vitamin D resistant rickets) | phosphate regulating | |
| | | endopeptidase homolog; | |
| | | phosphate regulating gene | |
| | | with homologies to | |
| | | endopeptidases on the X | |
| | | chromosome; phosphate | |
| | | regulating gene with | |
| | | homologies to endopeptidases | |
| | | on the X chromosome | |
| | | (hypophosphatemia, vitamin | |
| | | D resistant rickets) | |
| 117 | plasminogen activator, | tissue Plasminogen Activator | PLAT |
| | tissue | (tPA), T-PA, TPA, alteplase; | |
| | | plasminogen activator, tissue | |
| | | type; reteplase; t-plasminogen | |
| | | activator; tissue plasminogen | |
| | | activator (t-PA) | |
| 118 | proopiomelanocortin | Proopiomelanocortin; beta- | POMC |
| | (adrenocorticotropin/beta- | LPH; beta-MSH; alpha-MSH; | |
| | lipotropin/alpha- | gamma-LPH; gamma-MSH; | |
| | melanocyte stimulating | corticotropin; beta-endorphin; | |
| | hormone/beta-melanocyte | met-enkephalin; lipotropin | |
| | stimulating hormone/beta- | beta; lipotropin gamma; | |
| | endorphin) | melanotropin beta; N- | |
| | | terminal peptide; | |
| | | melanotropin alpha; | |
| | | melanotropin gamma; pro- | |
| | | ACTH-endorphin; | |
| | | adrenocorticotropin; pro- | |
| | | opiomelanocortin; | |
| | | corticotropin-lipotrophin; | |
| | | adrenocorticotropic hormone; | |
| | | alpha-melanocyte-stimulating | |
| | | hormone; corticotropin-like | |
| | | intermediary peptide | |
| 119 | periostin, osteoblast | Periostin-Like Factor; OSF-2, | POSTN |
| | specific factor | PDLPOSTN, PN, periostin, | |
| | | osteoblast specific factor 2 | |
| | | (fasciclin I-like); periodontal | |
| | | ligament-specific periostin | |
| 120 | peroxisome proliferative | Peroxisome proliferator- | PPARG |
| | activated receptor, gamma | activated receptor (PPAR), | |
| | | HUMPPARG, NR1C3, | |
| | | PPARG1, PPARG2, PPAR | |
| | | gamma; peroxisome | |
| | | proliferative activated | |
| | | receptor gamma; peroxisome | |
| | | proliferator activated-receptor | |
| | | gamma; peroxisome | |
| | | proliferator-activated receptor | |
| | | gamma 1; ppar gamma2 | |
| 121 | peptidylprolyl isomerase D | CYP 27C1, CYP-40, CYPD, | PPID |
| | (cyclophilin D) (is this the | 40 kDa peptidyl-prolyl cis- | |
| | right isoform?) | trans isomerase D; PPIase; | |
| | | cyclophilin 40; cyclophilin D; | |
| | | cyclophilin-related protein; | |
| | | peptidylprolyl isomerase D; | |
| | | rotamase | |
| 122 | peroxiredoxin 2 | NKEFB, PRP, PRXII, | PRDX2 |
| | | TDPX1, TSA, natural killer- | |
| | | enhancing factor B; thiol- | |
| | | specific antioxidant 1; | |
| | | thioredoxin peroxidase 1; | |
| | | thioredoxin-dependent | |
| | | peroxide reductase 1; torin | |
| 123 | prostaglandin- | Cyclo-oxygenase-2 (COX-2); | PTGS2 |
| | endoperoxide synthase 2 | COX-2, COX2, PGG/HS, | |
| | (prostaglandin G/H | PGHS-2, PHS-2, hCox-2, | |
| | synthase and | cyclooxygenase 2b; | |
| | cyclooxygenase) | prostaglandin G/H synthase | |
| | | and cyclooxygenase; | |
| | | prostaglandin-endoperoxide | |
| | | synthase 2 | |
| 124 | parathyroid hormone | PTH, parathormone; | PTH |
| | | parathyrin | |
| 125 | parathyroid hormone-like | parathyroid hormone related | PTHLH |
| | hormone | protein: PTH-related protein; | |
| | | humoral hypercalcemia of | |
| | | malignancy; osteostatin; | |
| | | parathyroid hormone-like | |
| | | protein; parathyroid | |
| | | hormone-like related protein; | |
| | | parathyroid hormone-related | |
| | | protein; parathyroid-like | |
| | | protein | |
| 126 | parathyroid hormone | parathyroid hormone receptor | PTHR1 |
| | receptor 1 | 1; PTHR, PTH receptor; | |
| | | PTH/PTHr receptor; | |
| | | PTH/PTHrP receptor; | |
| | | PTH/PTHrP type I receptor; | |
| | | parathyroid | |
| | | hormone/parathyroid | |
| | | hormone-related peptide | |
| | | receptor; parathyroid | |
| | | hormone/parathyroid | |
| | | hormone-related protein | |
| | | receptor; seven | |
| | | transmembrane helix receptor | |
| 127 | glutaminyl-peptide | GCT, QC, glutaminyl | QPCT |
| | cyclotransferase | cyclase; glutaminyl-peptide | |
| | (glutaminyl cyclase) | cyclotransferase | |
| 128 | retinal short chain | short-chain | RDHE2 |
| | dehydrogenase reductase | dehydrogenases/reductases | |
| | isoform 1 | (SDRs); RDH#2, RDH-E2, | |
| | | epidermal retinal | |
| | | dehydrogenase 2 | |
| 129 | regucalcin (senescence | RC, SMP30, regucalcin; | RGN |
| | marker protein-30) | senescence marker protein-30 | |
| 130 | runt-related transcription | AML3, CBFA1, CCD, | RUNX2 |
| | factor 2 | CCD1, OSF2, PEA2aA, | |
| | | PEBP2A1, PEBP2A2, | |
| | | PEBP2aA, PEBP2aA1, CBF- | |
| | | alpha 1; SL3-3 enhancer | |
| | | factor 1 alpha A subunit; | |
| | | SL3/AKV core-binding factor | |
| | | alpha A subunit; acute | |
| | | myeloid leukemia 3 protein; | |
| | | core-binding factor, runt | |
| | | domain, alpha subunit 1; | |
| | | osteoblast-specific | |
| | | transcription factor 2; | |
| | | polyomavirus enhancer | |
| | | binding protein 2 alpha A | |
| | | subunit | |
| 131 | S100 calcium binding | CABP1, CABP9K, CALB3, | S100G |
| | protein G | calbindin 3; calbindin 3, | |
| | | (vitamin D-dependent | |
| | | calcium binding protein); | |
| | | calbindin 3, (vitamin D- | |
| | | dependent calcium-binding | |
| | | protein); calbindin D9K | |
| 132 | serpin peptidase inhibitor, | plasminogen activator | SERPINE1 |
| | clade E (nexin, | inhibitor-1; PAI, PAI-1, | |
| | plasminogen activator | PAI1, PLANH1, plasminogen | |
| | inhibitor type 1), member 1 | activator inhibitor, type I; | |
| | | plasminogen activator | |
| | | inhibitor-1; serine (or | |
| | | cysteine) proteinase inhibitor, | |
| | | clade E (nexin, plasminogen | |
| | | activator inhibitor type 1), | |
| | | member 1 | |
| 133 | secreted frizzled related | secreted apoptosis-related | SFRP1 |
| | protein 1 | protein 2, FRP, FRP-1, FRP1, | |
| | | FrzA, SARP2, secreted | |
| | | apoptosis-related protein 2 | |
| 134 | sex hormone-binding | sex hormone-binding | SHBG |
| | globulin | globulin (SHBG), ABP, Sex | |
| | | hormone-binding globulin | |
| | | (androgen binding protein) | |
| 135 | SWI/SNF related, matrix | matrix associated, actin | SMARCC2 |
| | associated, actin dependent | dependent regulator of | |
| | regulator of chromatin, | chromatin | |
| | subfamily c, member 2 | | |
| 136 | sclerosteosis | VBCH, sclerostin | SOST |
| 137 | SRY (sex determining | SRY (sex determining region | SOX9 |
| | region Y)-box 9 | Y)-box 9 | |
| | (campomelic dysplasia, | | |
| | autosomal sex-reversal) | | |
| 138 | Sp7 transcription factor | OSX, osterix | SP7 |
| 139 | secreted protein, acidic, | ON, Osteonectin (secreted | SPARC |
| | cysteine-rich (osteonectin) | protein, acidic, cysteine-rich); | |
| | | cysteine-rich protein; | |
| | | osteonectin | |
| 140 | secreted phosphoprotein 1 | osteopontin: secreted | SPP1 |
| | (osteopontin, bone | phosphoprotein 1; secreted | |
| | sialoprotein I, early T- | phosphoprotein-1 | |
| | lymphocyte activation 1) | (osteopontin, bone | |
| | | sialoprotein) | |
| 141 | T-cell, immune regulator | ATP6N1C, ATP6V0A3, | TCIRG1 |
| | 1, ATPase, H+ | Atp6i, OC-116 kDa, OC116, | |
| | transporting, lysosomal V0 | OPTB1, Stv1, TIRC7, Vph1, | |
| | subunit A3 | a3, ATPase, H+ transporting, | |
| | | 116 kD; T cell immune | |
| | | response cDNA7 protein; T- | |
| | | cell, immune regulator 1; T- | |
| | | cell, immune regulator 1, | |
| | | ATPase, H+ transporting, | |
| | | lysosomal V0 protein A3; T- | |
| | | cell, immune regulator 1, | |
| | | ATPase, H+ transporting, | |
| | | lysosomal V0 protein a | |
| | | isoform 3; V-ATPase 116- | |
| | | kDa isoform a3; osteoclastic | |
| | | proton pump 116 kDa | |
| | | subunit; specific 116-kDa | |
| | | vacuolar proton pump | |
| | | subunit; vacuolar proton | |
| | | translocating ATPase 116 kDa | |
| | | subunit A isoform 3 | |
| 142 | transforming growth | TGF-beta: TGF-beta 1 | TGFB1 |
| | factor, beta 1 (Camurati- | protein; diaphyseal dysplasia | |
| | Engelmann disease) | 1, progressive; transforming | |
| | | growth factor beta 1; | |
| | | transforming growth factor, | |
| | | beta 1; transforming growth | |
| | | factor-beta 1, CED, DPD1, | |
| | | TGFB | |
| 143 | transforming growth | TGF beta 2; TGF-beta2 | TGFB2 |
| | factor, beta 2 | | |
| 144 | tumor necrosis factor (TNF | TNF-alpha (tumour necrosis | TNF |
| | superfamily, member 2) | factor-alpha); DIF, TNF- | |
| | | alpha, TNFA, TNFSF2, | |
| | | APC1 protein; TNF | |
| | | superfamily, member 2; TNF, | |
| | | macrophage-derived; TNF, | |
| | | monocyte-derived; cachectin; | |
| | | tumor necrosis factor alpha | |
| 145 | tumor necrosis factor | CD265, EOF, FEO, ODFR, | TNFRSF11A |
| | receptor superfamily, | OFE, PDB2, RANK, | |
| | member 11a, NFKB | TRANCER, osteoclast | |
| | activator | differentiation factor | |
| | | receptor; receptor activator of | |
| | | nuclear factor-kappa B; tumor | |
| | | necrosis factor receptor | |
| | | superfamily, member 11a; | |
| | | tumor necrosis factor receptor | |
| | | superfamily, member 11a, | |
| | | activator of NFKB | |
| 146 | tumor necrosis factor | OPG (osteoprotegerin), | TNFRSF11B |
| | receptor superfamily, | OCIF, OPG, TR1, | |
| | member 11b | osteoclastogenesis inhibitory | |
| | (osteoprotegerin) | factor; osteoprotegerin | |
| 147 | tumor necrosis factor | soluble necrosis factor | TNFRSF1B |
| | receptor superfamily, | receptor; CD120b, TBPII, | |
| | member 1B | TNF-R-II, TNF-R75, | |
| | | TNFBR, TNFR2, TNFR80, | |
| | | p75, p75TNFR, p75 TNF | |
| | | receptor; tumor necrosis | |
| | | factor beta receptor; tumor | |
| | | necrosis factor binding | |
| | | protein 2; tumor necrosis | |
| | | factor receptor 2 | |
| 148 | tumor necrosis factor | RANKL; CD254, ODF, | TNFSF11 |
| | (ligand) superfamily, | OPGL, RANKL, TRANCE, | |
| | member 11 | hRANKL2, sOdf, TNF- | |
| | | related activation-induced | |
| | | cytokine; osteoclast | |
| | | differentiation factor; | |
| | | osteoprotegerin ligand; | |
| | | receptor activator of nuclear | |
| | | factor kappa B ligand; tumor | |
| | | necrosis factor ligand | |
| | | superfamily, member 11 | |
| 149 | tenascin W | tenw, zgc: 110729 | tnw |
| 150 | TNF receptor-associated | RNF85 | TRAF6 |
| | factor 6 | | |
| 151 | thioredoxin interacting | thioredoxin binding protein 2; | TXNIP |
| | protein | upregulated by 1,25- | |
| | | dihydroxyvitamin D-3 | |
| 152 | TYRO protein tyrosine | DNAX-activating protein 12; | TYROBP |
| | kinase binding protein | DAP12, KARAP, PLOSL, | |
| | | DNAX-activation protein 12; | |
| | | killer activating receptor | |
| | | associated protein | |
| 153 | ubiquitin-conjugating | E2(17)KB2, PUBC1, UBC4, | UBE2D2 |
| | enzyme E2D 2 (UBC4/5 | UBC4/5, UBCH5B, ubiquitin | |
| | homolog, yeast) | carrier protein; ubiquitin- | |
| | | conjugating enzyme E2 D2 | |
| | | transcript variant 1; ubiquitin- | |
| | | conjugating enzyme E2-17 kDa | |
| | | 2; ubiquitin-conjugating | |
| | | enzyme E2D 2; ubiquitin- | |
| | | conjugating enzyme E2D 2 | |
| | | (homologous to yeast | |
| | | UBC4/5) | |
| 154 | vitamin D (1,25- | vitamin D receptor 1; NR1I1; | VDR |
| | dihydroxyvitamin D3) | vitamin D (1,25- | |
| | receptor | dihydroxyvitamin D3) | |
| | | receptor | |
| 155 | vascular endothelial | VEGF; VEGFA, VPF, | VEGF |
| | growth factor | vascular endothelial growth | |
| | | factor A; vascular | |
| | | permeability factor | |
| 156 | wingless-type MMTV | Wnt16 | WNT16 |
| | integration site family, | | |
| | member 16 | | |
| 157 | Werner syndrome | RECQ3, RECQL2, RECQL3, | WRN |
| | | Werner Syndrome helicase; | |
| | | Werner syndrome protein | |
| 158 | IgA antigliadin antibodies | IgA antigliadin antibodies | AGA |
| | (AGA) | (AGA) | |
| 159 | calcium | ionized calcium | CALCIUM |
| 160 | CD8 T cells lacking CD28 | CD8 T cells lacking CD28 | CD8T |
| | expression | expression | |
| 161 | dehydroepiandrosterone | dehydroepiandrosterone | DHEAS |
| | sulfate (DHEAS) | sulfate (DHEAS), | |
| 162 | deoxypyridinoline | deoxypyridinoline (Dpyr)- | DPYR |
| | | urine; DpD | |
| 163 | serum IgA endomysial | serum IgA endomysial | EMAIgA |
| | antibody (EMA) | antibody (EMA) | |
| 164 | estradiol | Estradiol; 17b- | ESTRA |
| | | estradiol; 1,3,5[10]- | |
| | | estratriene-3,17b-diol; 3,17b- | |
| | | Dihydroxy-1,3,5[10]- | |
| | | estratriene; Estra-1,3,5(10)- | |
| | | triene-3,17-diol; Beta- | |
| | | estradiol | |
| 165 | 17-beta-estradiol | 17-beta-estradiol inducible | EstraCIF |
| | inducible caspase-6 | caspase-6 inhibitory factor. | |
| | inhibitory factor | | |
| 166 | estrogen | estrogen | ESTROGEN |
| 167 | collagen 1 alpha 1 | HELP | HELP |
| | helicoidal peptide | | |
| 168 | hydroxylysine-glycosides | HYLG; GGHL, GHL | GGHL |
| 169 | Hydroxyproline | Hydroxyproline, total and | OHP |
| | | dialyzable; OHP, Hyp | |
| 170 | homocysteine | Homocysteine (total) | HOMOCYST |
| 171 | carboxy-terminal- | collagen I degradation | ICTP |
| | telopeptide of type I | byproduct (ICTP), carboxy- | |
| | collagen (ICTP) | terminal-telopeptide of type I | |
| | | collagen (ICTP); CTX-I; | |
| | | CTXI; CTX-MMP | |
| 172 | INSP078 | insp078 | INSP078 |
| 173 | INTP009 | intp009 | INTP009 |
| 174 | M component | M component (monoclonal | MCOMP |
| | | bands) | |
| 175 | nitric oxide | nitric oxide | NO |
| 176 | atrial natriuretic peptide | ATRIAL NATRIURETIC | NPPACR |
| | clearance receptor variant | PEPTIDE CLEARANCE | |
| | | RECEPTOR VARIANT | |
| 177 | N-terminal crosslinking | N-terminal crosslinking | NTX1 |
| | telopeptide of type 1 | telopeptide of type 1 collagen | |
| | collagen | | |
| 178 | osteometrin | osteometrin | OMETRN |
| 179 | osteoblastic stem cell | osteoblastic stem cell factor | OSCF |
| | factor | | |
| 180 | pancreas-derived factor | pancreas-derived factor seq id | PDF1 |
| | SEQ ID NO: 1 | no: 1 | |
| 181 | prostaglandin E2 | PGE₂, prostaglandin E2 | PGE2 |
| 182 | C terminal propeptide of | C terminal propeptide of | PICP |
| | Type 1 procollagen (PICP) | Type 1 procollagen (PICP), | |
| | | CICP, collagen I synthesis | |
| | | byproduct (PICP) | |
| 183 | collagen III synthesis | collagen III synthesis | PIIINP |
| | byproduct (PIIINP) | byproduct (PIIINP) | |
| 184 | amino-terminal propeptide | Amino-terminal propeptide of | PINP |
| | of type I procollagen | type I procollagen (PINP), | |
| | (PINP) | collagen I synthesis | |
| | | byproduct (PINP) | |
| 185 | polyamines (putrescine, | polyamines (putrescine, | POLYAMINE |
| | spermidine, spermine) | spermidine, spermine) | |
| 186 | pyridinoline | pyridinoline | PYRID |
| 187 | vitamin D3 | vitamin D3 | VitD3 |
| 188 | vitamin K | vitamin K | VitK |
| 189 | vitamin K homologues | vitamin K homologues | VitKhomo |
| | | including phylloquinone | |
| | | (PK), menaquinone-4 (MK- | |
| | | 4), and menaquinone-7 (MK- | |
| | | 7), K2 | |
| 190 | 17906 gene from | 17906 gene from Millenium | |
| | Millenium | | |
| 191 | BAA83099.1, | baa83099.1, aad46161.1, | |
| | AAD46161.1, | aad38507.2 and acc4 | |
| | AAD38507.2 and ACC4 | | |

Included as an aspect of the invention are several methods of constructing panels from sub-sets of the complete set of OSTEORISKMARKERS listed above. One skilled in the art will note that the above listed OSTEORISKMARKERS come from a diverse set of molecular pathways and physiological functions, and may also be clustered into groupings by virtue of their direct and indirect interactions and correlation with each other, including those summarized by their relative position on a canonical molecular pathway.

FIG. 1A-1AA are graphic illustrations of the many canonical molecular pathways listed within the Kyoto University Encyclopedia of Genes and Genomes (KEGG) which feature three or more OSTEORISKMARKERS, identified by their common HUGO gene name abbreviation or alias (or other group abbreviation when multiple similar biomarkers are shown), in each disclosed canonical pathway. FIG. 2 is a listing of KEGG pathways with one or two OSTEORISKMARKERS identified as contained within them. Panels of OSTEORISKMARKERS may be constructed by selecting one or more of the OSTEORISKMARKERS indicated across one or more KEGG pathways so as to select a desired measurement of the molecular activity within the pathway, and across several relevant pathways. Several KEGG pathways may thus be simultaneously assessed, providing broader perspective of the molecular physiology of various aspects of bone metabolism in a subject.

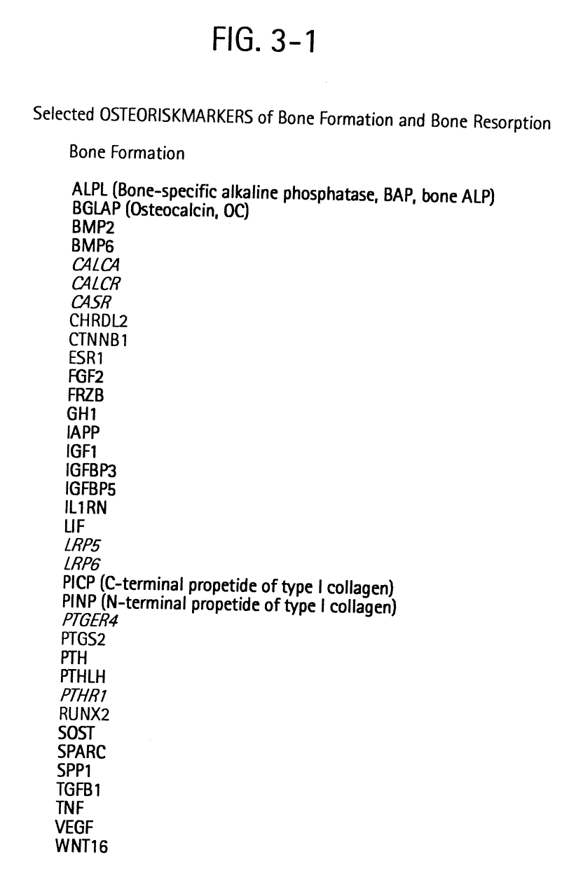

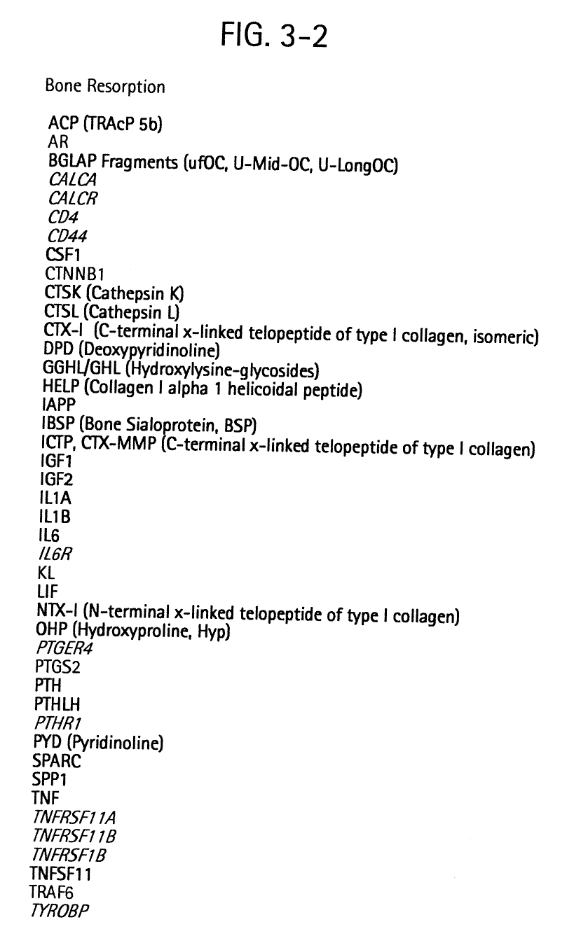

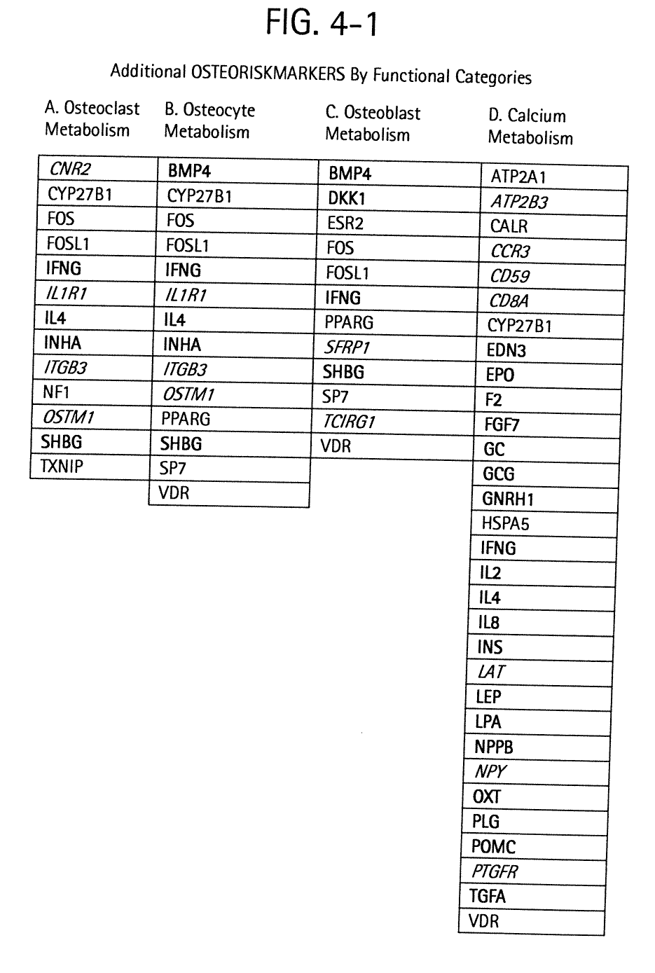

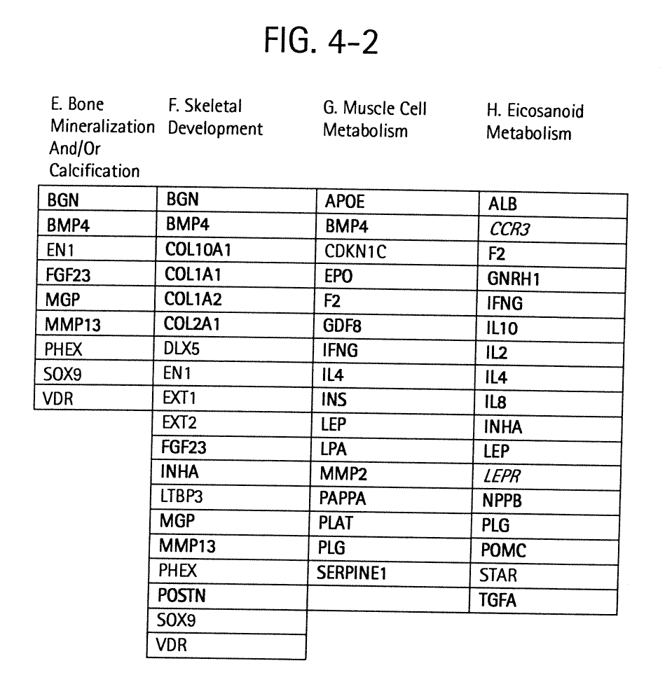

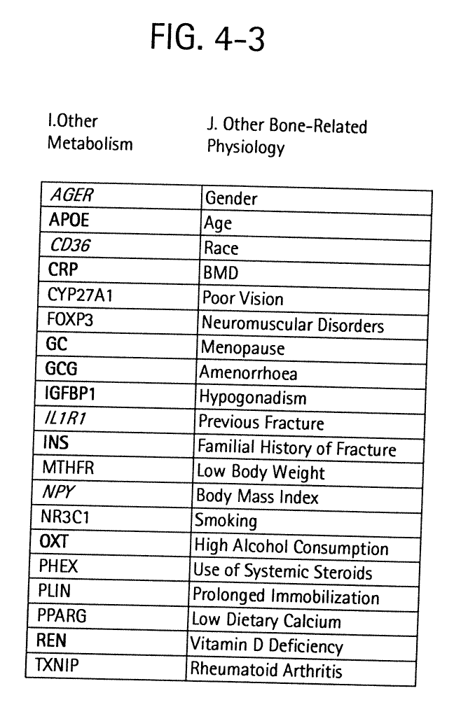

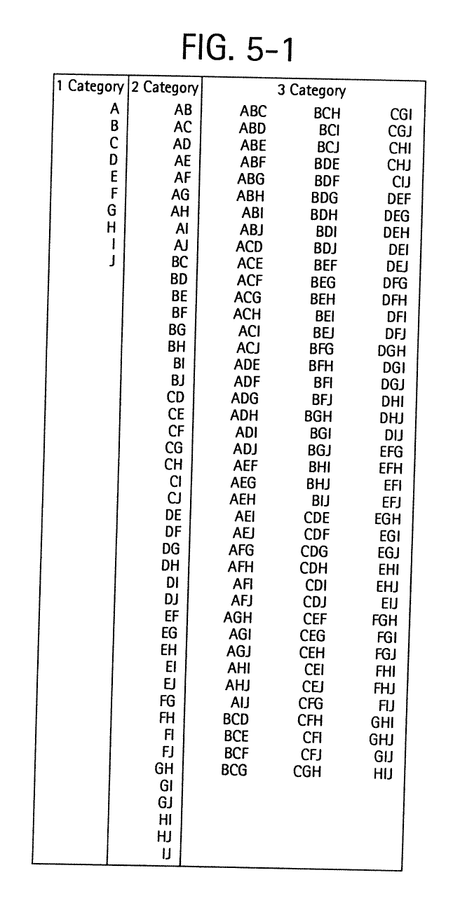

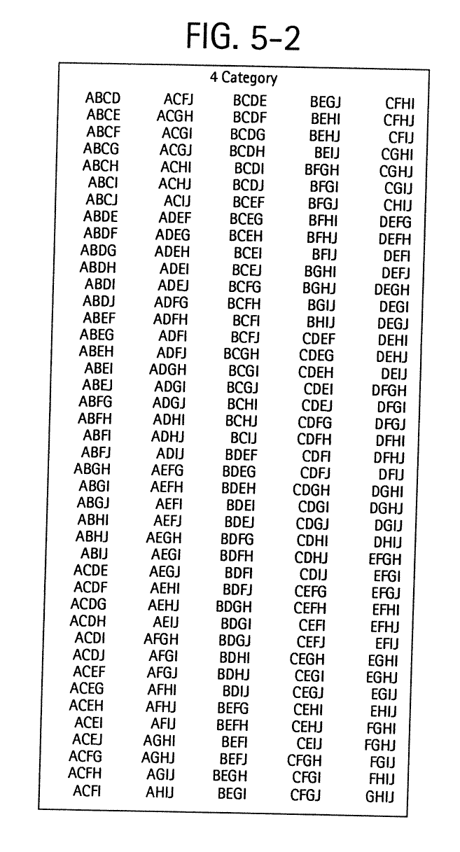

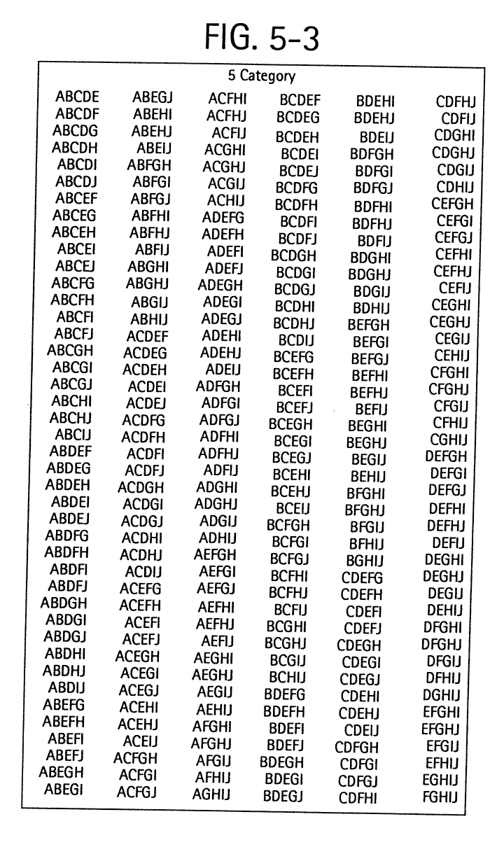

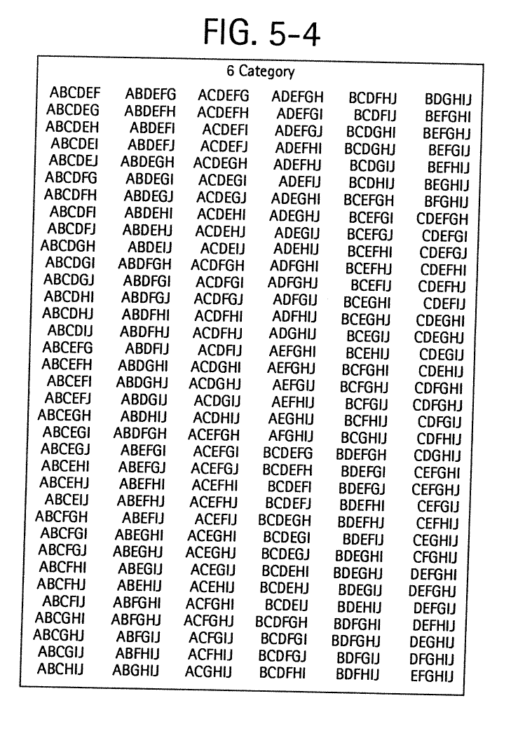

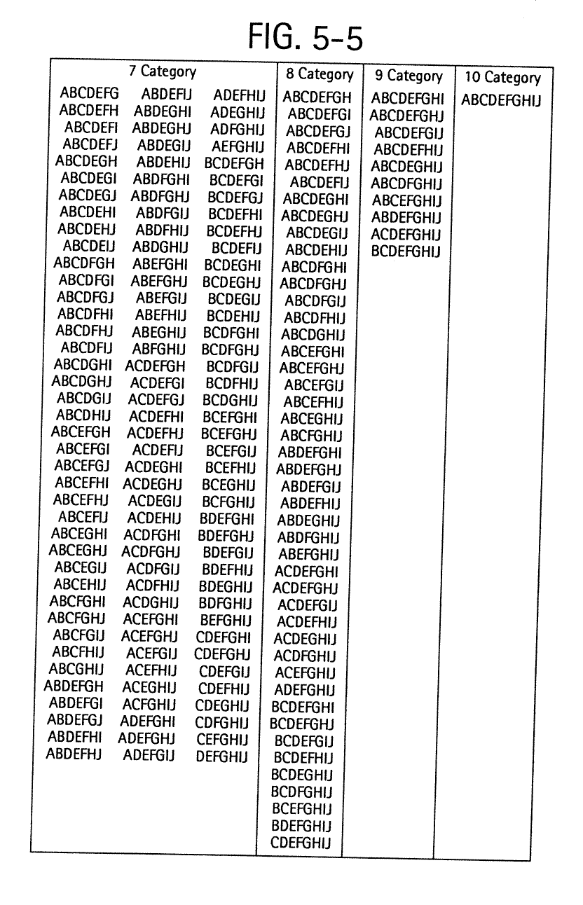

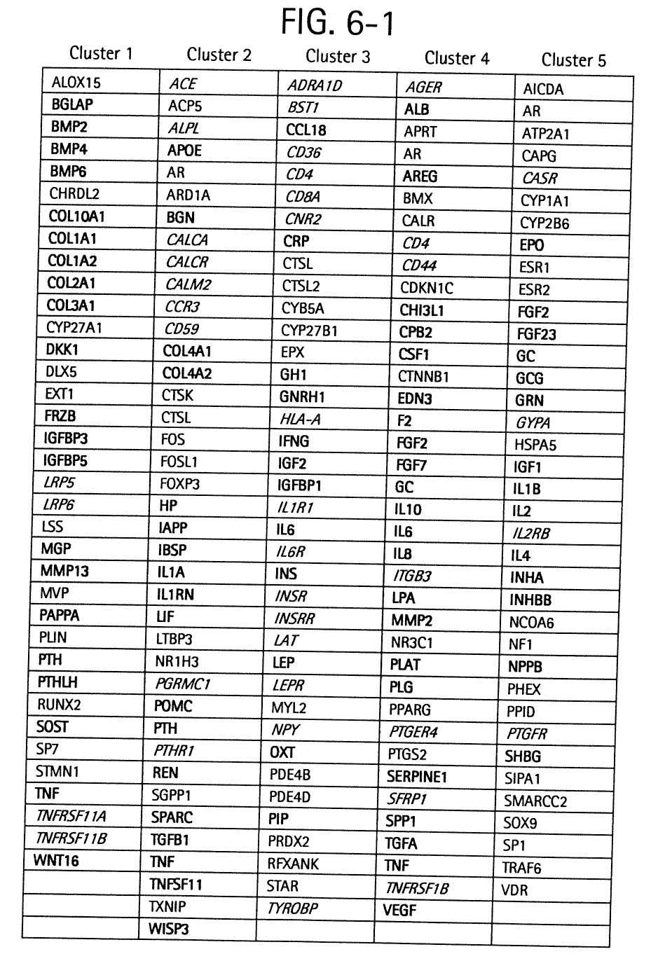

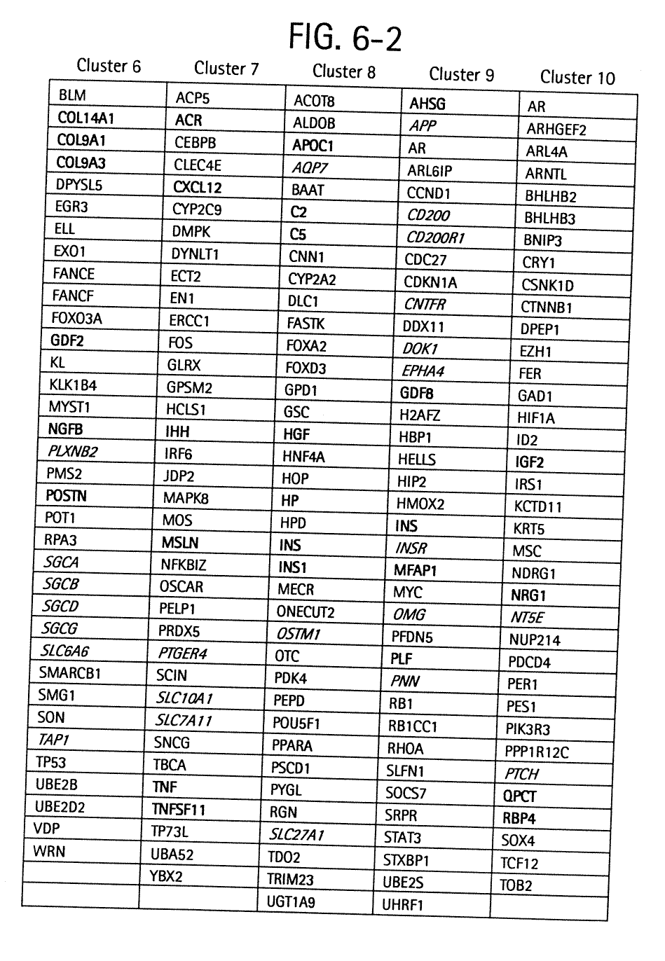

OSTEORISKMARKERS may also be grouped according to the physiological functions in which they are implicated or with which they are associated. A common division and characterization of the physiological functions within the bone multicellular unit or BMU is between that of bone resorption (typically related to the activity of osteoclasts) and that of bone formation (typically related to the activity of osteoblasts). A reduction in bone density, such as that seen in osteoporosis or pre-osteoporosis, results when these two physiological activities are not in balance. FIG. 3 is a table listing individual OSTEORISKMARKERS divided into two categories based on their association with the physiological functions of bone formation (left column) and of bone resorption (right column). OSTEORISKMARKERS which are commonly found localized in the extracellular space or plasma membranes of cells are also highlighted in bold or italics, respectively, in this and the following Figures. Of particular note is that many of the disclosed OSTEORISKMARKERS shown in FIG. 3 are associated with bone formation and resorption, or come from common precursors, as is true of the large number of collagen related OSTEORISKMARKERS (where the specific OSTEORISKMARKER may be a pre-cursor or degradation product of collagen). Specific panels of OSTEORISKMARKERS may be constructed based on selecting one or more OSTEORISKMARKERS from each of either one or both categories shown (formation and resorption).

In addition to the general OSTEORISKMARKERS that can be categorized according to FIG. 3, additional OSTEORISKMARKERS can be listed according to physiological functions. FIG. 4 is a table listing additional individual OSTEORISKMARKERS categorized by their association with the following ten physiological functions: osteoclast metabolism (category A), osteocyte metabolism (category B), osteoblast metabolism (category C), calcium metabolism (category D), bone ossification or mineralization (category E), skeletal development (category F), muscle cell metabolism (including the proliferation and movement of muscle cells, including vascular and vascular smooth muscle cells; category G), eicosanoid metabolism (category H), other metabolism (category I), and other bone-related physiology (category J). As in the earlier categorization, many individual OSTEORISKMARKERS are represented in more than one physiological function and category.

One or more OSTEORISKMARKER(S) from each of one or more physiological function associated categories from FIG. 4 may be combined together into panels of biomarkers according to the invention. FIG. 5 is a table listing various combinations useful in constructing panels of the additional OSTEORISKMARKERS from FIG. 4. Each set of one to ten letters indicate a class of OSTEORISKMARKER panel, and indicates the use of one or more markers each from one or more of the previously mentioned categories. Representative examples of OSTEORISKMARKER panels according to this method of the invention are also hereby explicitly disclosed in the tables of FIG. 5, where a given letter abbreviation shown in the panel indicates that one or more OSTEORISKMARKERS are chosen from the OSTEORISKMARKERS listed in that appropriate physiological function's category in the preceding FIG. 4 when constructing such a panel.

In further embodiments of the invention, these additional OSTEORISKMARKER combination panels shown in FIG. 4 may themselves be further combined with one or more OSTEORISKMARKER(S) selected from either one or both of the general categories of bone formation and of bone resorption, respectively, previously identified in FIG. 3, yielding up to twelve physiological function categories represented in a given panel according the invention.

OSTEORISKMARKERS may also be categorized into groups based on their closeness, either in a canonical molecular pathway, or as proven experimentally to interact or correlate with one another. FIG. 6 is a table listing eleven clusters of OSTEORISKMARKERS grouped by their relative position, interactions, correlations and network proximity as defined by protein-protein interactions and through participation in one or more canonical pathways, presented in the figure together with their near neighbors and interaction partners within pathways. As in the earlier categorizations, many individual OSTEORISKMARKERS are represented in more than one cluster. OSTEORISKMARKER panels may also be constructed by means of selection of one or more OSTEORISKMARKERS each from one or more of the eleven clusters listed in FIG. 6.

OSTEORISKMARKERS may be further selected by virtue of their cell localization. OSTEORISKMARKERS which are commonly found localized in the extracellular space or plasma membranes of cells are also highlighted in bold or italics, respectively.

One skilled in the art will realize that panels can also be made of combinations of these techniques, where individual OSTEORISKMARKERS are chosen from a molecular pathway, a physiological function categorization, or from a cluster shown in the previous Figures. Additionally, each of the OSTEORISKMARKER panels previously discussed may itself be combined with any one or more individual OSTEORISKMARKER(S) listed in Table 1, or their functional or statistical equivalent (as herein defined), where said OSTEORISKMARKER is not categorized elsewhere in the Figures.

The above discussion for convenience focuses on a subset of the OSTEORISKMARKERS; other OSTEORISKMARKERS and even biomarkers which are not listed in the above table but related to these physiological functions and molecular pathways may prove to be useful given the signal and information provided from these studies. To the extent that other participants within the total list of OSTEORISKMARKERS are also relevant functional or molecular participants in osteoporosis, osteopenia and pre-osteoporosis, they may be functional equivalents to the biomarkers thus far disclosed and therefore themselves be OSTEORISKMARKERS, provided they additionally share certain defined characteristics of a good biomarker, which would include both this biological process involvement and also analytically important characteristics such as the bioavailability of said markers at a useful signal to noise ration, and in a useful sample matrix such as blood serum. Such requirements typically limit the usefulness of many members of a biological KEGG pathway, as this is unlikely to be generally the case, and frequently occurs only in pathway members that constitute secretory substances, and thus are found to be extracellular, those accessible on the plasma membranes of cells, which may be released or accessible by extracellular means, as well as those that are released into the serum upon cell death, due to apoptosis or for other reasons such as bone unit remodeling or other cell turnover or cell necrotic processes, whether or not said is related to the disease progression of pre-osteoporosis and osteoporosis. Furthermore, the statistical utility of such additional OSTEORISKMARKERS is substantially dependent on the cross-correlation between markers and new markers will often be required to operate within a panel in order to elaborate the meaning of the underlying biology. A biomarker is considered statistically equivalent when levels of the new biomarker are well correlated with a previously disclosed OSTEORISKMARKER, through the progression of the pre-disease and disease, and across the appropriate range of the risk. However, the remaining and future biomarkers that meet this high standard for OSTEORISKMARKERS are likely to be quite valuable. Our invention encompasses such functional and statistical equivalents to the aforelisted OSTEORISKMARKERS.

As is shown in FIGS. 1, 2, and 6, many OSTEORISKMARKERS are closely correlated and clustered in molecular pathway groups, physiological functions, or in clusters that thus rise or fall in their concentration with each other (or in opposite directions to each other). While this may offer multiple opportunities for new and useful OSTEORISKMARKERS within known and previously disclosed biological pathways, our invention hereby anticipates and claims such useful biomarkers that are functional or statistical equivalents to those listed, and such correlations and the potential identities of other biological pathway members are disclosed in the aforementioned figures.

The OSTEORISKMARKERS herein disclosed are also useful in the differential diagnosis of various bone diseases and their causes, or to indicate an endogenous or exogenous cause for osteoporosis, osteopenia or pre-osteoporosis. Individuals who are diagnosed with osteoporosis often do so as a byproduct of another condition or medication use. In fact, there are a wide variety of diseases along with certain medications and toxic agents that can cause or contribute to the development of osteoporosis. Individuals who get the disease due to these “outside” causes are said to have “secondary” osteoporosis. They typically experience greater levels of bone loss than would be expected for a normal individual of the same age, gender, and race.

Several genetic diseases have been linked to secondary osteoporosis. Idiopathic hyper-calciuria and cystic fibrosis are the most common. Patients with cystic fibrosis have markedly decreased bone density and increased fracture rates due to a variety of factors, including calcium and vitamin D malabsorption, reduced sex steroid production and delayed puberty, and increased inflammatory cytokines. Some patients with idiopathic hypercalciuria have a renal defect in the ability of the kidney to conserve calcium. Several studies have documented low bone density in these individuals.

Estrogen or testosterone deficiency during adolescence (due to Turner's, Kallman's, or Klinefelter's syndrome, anorexia nervosa, athletic amenorrhea, cancer, or any chronic illness that interferes with the onset of puberty) leads to low peak bone mass. Estrogen deficiency that develops after peak bone mass is achieved but before normal menopause (due to premature ovarian failure for example) is associated with rapid bone loss. Low sex steroid levels may also be responsible for reduced bone density in patients with androgen insensitivity or acromegaly. By contrast, excess thyroid hormone (thyrotoxicosis), whether spontaneous or caused by overtreatment with thyroid hormone, may be associated with substantial bone loss; while bone turnover is increased in these patients, bone resorption is increased more than bone formation. Likewise, excess production of glucocorticoids caused by tumors of the pituitary or adrenal glands (Cushing's syndrome) can lead to rapidly progressive and severe osteoporosis, as can treatment with glucocorticoids. Primary hyperparathyroidism is a relatively common condition in older individuals, especially postmenopausal women, that is caused by excessive secretion of parathyroid hormone. Most often, the cause is a benign tumor (adenoma) in one or more parathyroid glands; very rarely (less than 0.5 percent of the time) the cause is parathyroid cancer.

Diseases that reduce intestinal absorption of calcium and phosphorus, or impair the availability of vitamin D, can also cause bone disease. Moderate malabsorption results in osteoporosis, but severe malabsorption may cause osteomalacia. Celiac disease, due to inflammation of the small intestine by ingestion of gluten, is an important and commonly overlooked cause of secondary osteoporosis. Likewise, osteoporosis and fractures have been found in patients following surgery to remove part of the stomach (gastrectomy), especially in women. Bone loss is seen after gastric bypass surgery even in morbidly obese women who do not have low bone mass initially. Increased osteoporosis and fractures are also seen in patients with Crohn's disease and ulcerative colitis. Glucocorticoids, commonly used to treat both disorders, probably contribute to the bone loss. Similarly, diseases that impair liver function (primary biliary cirrhosis, chronic active hepatitis, cirrhosis due to hepatitis B and C, and alcoholic cirrhosis) may result in disturbances in vitamin D metabolism and may also cause bone loss by other mechanisms. Primary biliary cirrhosis is associated with particularly severe osteoporosis. Fractures are more frequent in patients with alcoholic cirrhosis than any other types of liver disease, although this may be related to the increased risk of falling among heavy drinkers. Human immunodeficiency virus (HIV) infected patients also have a higher prevalence of osteopenia or osteoporosis. This may involve multiple endocrine, nutritional, and metabolic factors and may also be affected by the antiviral therapy that HIV patients receive.

Autoimmune and allergic disorders are associated with bone loss and increased fracture risk. This is due not only to the effect of immobilization and the damage to bone by the products of inflammation from the disorders themselves, but also from the glucocorticoids that are used to treat these conditions. Rheumatic diseases like lupus and rheumatoid arthritis have both been associated with lower bone mass and an increased risk of fractures.

Many neurologic disorders are associated with impaired bone health and an increased risk of fracture. This may be due in part to the effects of these disorders on mobility and balance or to the effects of drugs used in treating these disorders on bone and mineral metabolism. For example, patients with stroke, spinal cord injury, or neurologic disorders show rapid bone loss in the affected areas. There are many disabling conditions that can lead to bone loss, such as cerebral palsy, as well as diseases affecting nerve and muscle, such as poliomyelitis and multiple sclerosis. Children and adolescents with these disorders are unlikely to achieve optimal peak bone mass, due both to an increase in bone resorption and a decrease in bone formation. In some cases very rapid bone loss can produce a large enough increase in blood calcium levels to produce symptoms. Fractures are common in these individuals not only because of bone loss, but also because of muscular weakness and neurologic impairment that increases the likelihood of falls. Bone loss can be slowed—but not completely prevented—by antiresorptive therapy. Epilepsy is another neurologic disorder that increases the risk of bone disease, primarily because of the adverse effects of anti-epileptic drugs. Many of the drugs used in epilepsy can impair vitamin D metabolism, probably by acting on the liver enzyme which converts vitamin D to 25 hydroxy vitamin D. In addition, there may be a direct effect of these agents on bone cells. Due to the negative bone-health effects of drugs, most epilepsy patients are at risk of developing osteoporosis. In those who have low vitamin D intakes, intestinal malabsorption, or low sun exposure, the additional effect of anti-epileptic drugs can lead to osteomalacia.

Psychiatric disorders can also have a negative impact on bone health. While anorexia nervosa is the psychiatric disorder that is most regularly associated with osteoporosis, major depression, a much more common disorder, is also associated with low bone mass and an increased risk of fracture. Many studies show lower BMD in depressed patients. Higher scores for depressive symptoms have also been reported in women with osteoporosis. Yet what these studies do not make clear is whether major depression causes low BMD and increased fracture risk, or whether the depression is a consequence of the diminished quality of life and disability that occurs in many osteoporotic patients. One factor that may cause bone loss in severely depressed individuals is increased production of cortisol, the adrenal stress hormone. Whatever the cause of low BMD and increased fracture risk, measurement of BMD is appropriate in both men and women with major depression. While the response of individuals with major depression to calcium, vitamin D, or antiresorptive therapy has not been specifically documented, it would seem reasonable to provide these preventive measures to patients at high risk.

Aseptic necrosis (also called osteonecrosis or avascular necrosis) is a well-known skeletal disorder that may be a complication of injury, treatment with glucocorticoids, or alcohol abuse. Chronic obstructive pulmonary disease (emphysema and chronic bronchitis) is also now recognized as being associated with osteoporosis and fractures even in the absence of glucocorticoid therapy. Immobilization is clearly associated with rapid bone loss; patients with spinal cord lesions are at particularly high risk for fragility fractures. However, even modest reductions in physical activity can lead to bone loss. Hematological disorders, particularly malignancies, are commonly associated with osteoporosis and fractures as well.

Osteoporosis can also be a side effect of particular medical therapies. Glucocorticoid-Induced Osteoporosis (GIO) is a common form of osteoporosis produced by drug treatment. With the increased use of prednisone and other drugs that act like cortisol for the treatment of many inflammatory and autoimmune diseases, this form of bone loss has become a major clinical concern. The concern is greatest for those diseases in which the inflammation itself and/or the immobilization caused by the illness also caused increased bone loss and fracture risk. Glucocorticoids, which are used to treat a wide variety of inflammatory conditions (e.g., rheumatoid arthritis, asthma, emphysema, chronic lung disease), can cause profound reductions in bone formation and may, to a lesser extent, increase bone resorption, leading to loss of trabecular bone at the spine and hip, especially in postmenopausal women and older men. The most rapid bone loss occurs early in the course of treatment, and even small doses (equivalent to 2.5-7.5 mg prednisone per day) are associated with an increase in fractures. The risk of fractures increases rapidly in patients treated with glucocortocoids, even before much bone has been lost. This rapid increase in fracture risk is attributed to damage to the bone cells, which results in less healthy bone tissue.

Cyclosporine A and tacrolimus are widely used in conjunction with glucocorticoids to prevent rejection after organ transplantation, and high doses of these drugs are associated with a particularly severe form of osteoporosis. Bone disease has also been reported with several frequently prescribed anticonvulsants, including diphenylhydantoin, phenobarbital, sodium valproate, and carbamazepine. Patients who are most at risk of developing this type of bone disease include those on long-term therapy, high medication doses, multiple anticonvulsants, and/or simultaneous therapy with medications that raise liver enzyme levels. Low vitamin D intake, restricted sun exposure, and the presence of other chronic illnesses increase the risk, particularly among elderly and institutionalized individuals. In contrast, high intakes of vitamin A (retinal) may increase fracture risk. Methotrexate, a folate antagonist used to treat malignancies and (in lower doses) inflammatory diseases such as rheumatoid arthritis, may also cause bone loss, although research findings are not consistent. In addition, gonadotropin-releasing hormone (GnRH) agonists, which are used to treat endometriosis in women and prostate cancer in men, reduce both estrogen and testosterone levels, which may cause significant bone loss and fragility fractures.

Rickets (which affects children) and osteomalacia (which affects adults) are conditions that can result from a delay in depositing calcium phosphate mineral in growing bones, thus leading to skeletal deformities, especially bowed legs. In adults, the equivalent disease is called osteomalacia. Since longitudinal growth has stopped in adults, deficient bone mineralization does not cause skeletal deformity but can lead to fractures, particularly of weight-bearing bones such as the pelvis, hip, and feet. Even when there is no fracture, many patients with rickets and osteomalacia suffer from bone pain and can experience severe muscle weakness. Rickets and osteomalacia are typically caused by any of a variety of environmental abnormalities. While rare, the disorder can also be inherited as a result of mutations in the gene producing the enzyme that converts 25-hydroxy vitamin D to the active form, 1,25-dihydroxy vitamin D, or in the gene responsible for the vitamin D receptor. Osteomalacia can also be caused by disorders that cause marked loss of phosphorus from the body. This can concur as a congenital disorder or can be acquired in patients who have tumors that produce a protein that affects phosphorus transport in the kidney.

There is also a second form of rickets and osteomalacia that is caused by phosphate deficiency. This condition can be inherited (also known as X-linked hypophosphatemic rickets), but it is more commonly the result of other factors. Individuals with diseases affecting the kidney's ability to retain phosphate rapidly are at risk of this condition, as are those with diseases of the renal tubule that affect the site of phosphate reabsorption. While most foods are rich in phosphate, phosphate deficiency may also result from consumption of very large amounts of antacids containing aluminum hydroxide, which prevents the absorption of dietary phosphate. Rickets due to phosphate deficiency can also occur in individuals with acquired or inherited defects in acid secretion by the kidney tubule and those who take certain drugs that interfere with phosphate absorption or the bone mineralization process. There are also patients who develop tumors that secrete a factor that causes loss of phosphate from the body. This condition is called tumor-induced or oncogenic osteomalacia.

Patients with chronic renal disease are not only at risk of developing rickets and osteomalacia, but they are also at risk of a complex bone disease known as renal osteodystrophy. This condition is characterized by a stimulation of bone metabolism caused by an increase in parathyroid hormone and by a delay in bone mineralization that is caused by decreased kidney production of 1,25-dihydroxyvitamin D. In addition, some patients show a failure of bone formation, called adynamic bone disease.

Paget's disease of bone is a progressive, often crippling disorder of bone remodeling that commonly involves the spine, pelvis, legs, or skull (although any bone can be affected). If diagnosed early, its impact can be minimized. Individuals with this condition experience an increase in bone loss at the affected site due to excess numbers of overactive osteoclasts. While bone formation increases to compensate for the loss, the rapid production of new bone leads to a disorganized structure. The resulting bone is expanded in size and associated with increased formation of blood vessels and connective tissue in the bone marrow. Such bone becomes more susceptible to deformity or fracture. Depending on the location, the condition may produce no clinical signs or symptoms, or it may be associated with bone pain, deformity, fracture, or osteoarthritis of the joints adjacent to the abnormal bone. Paget's disease of bone can also cause a variety of neurological complications as a result of compression of nerve tissue by pagetic bone. In very rare cases (probably less than 1 percent of the time) the disease is complicated by the development of an osteosarcoma.

A large number of genetic and developmental disorders affect the skeleton. Among the more common and more important of these is a group of inherited disorders referred to as osteogenesis imperfecta or OI. Patients with this condition have bones that break easily (therefore, the condition is also known as brittle bone disease). There are a number of forms of OI that result from different types of genetic defects or mutations. These defects interfere with the body's production of type I collagen, the underlying protein structure of bone. Most, but not all, forms of OI are inherited. The disease manifests through a variety of clinical signs and symptoms, ranging from severe manifestations that are incompatible with life (that is, causing a stillbirth) to a relatively asymptomatic disease. However, most OI patients have low bone mass (osteopenia) and as a result suffer from recurrent fractures and resulting skeletal deformities. There are four main types of OI, which vary according to the severity and duration of the symptoms. The most common form (Type I) is also the mildest version; and patients may have relatively few fractures. The second mildest form of the disease (which is called Type IV, because it was the fourth type of OI to be discovered) results in mild to moderate bone deformity, and sometimes in dental problems and hearing loss. These patients also sometimes have a blue, purple, or gray discoloration in the whites of their eyes, a condition known as blue sclera. A more severe form of the disease (Type III) results in relatively frequent fractures, and often in short stature, hearing loss, and dental problems. Finally, patients with the most severe form of the disease (Type II) typically suffer numerous fractures and severe bone deformity, generally leading to early death.

A large group of rare diseases (sclerosing bone disorders) can cause an increase in bone mass. Instead of overactive osteoclasts, osteopetrosis results from a variety of genetic defects that impair the ability of osteoclasts to resorb bone. This interferes with the normal development of the skeleton and leads to excessive bone accumulation. Although such bone is very dense, it is also brittle and thus fractures often result. In addition, by compressing various nerves, the excess bone in patients with osteopetrosis may cause neurological symptoms, such as deafness or blindness. These patients may also suffer anemia, as blood-forming cells in the bone marrow are “crowded out” by the excess bone. Similar symptoms can result from over-activity of these bone cells, as in fibrous dysplasia where bone-forming cells produce too much connective tissue.

Bone tumors can originate in the bone (these are known as primary tumors) or, much more commonly, result from the seeding of bone by tumors outside of the skeleton (these are known as metastatic tumors, since they have spread from elsewhere). Both types of tumors can destroy bone, although some metastatic tumors can actually increase bone formation. Primary bone tumors can be either benign (noncancerous) or malignant (cancerous). The most common benign bone tumor is osteochondroma, while the most common malignant ones are osteosarcoma and Ewing's sarcoma. Metastatic tumors are often the result of breast or prostate cancer that has spread to the bone. These may destroy bone (osteolytic lesion) or cause new bone formation (osteoblastic lesion). Breast cancer metastases are usually osteolytic, while most prostate cancer metastases are osteoblastic, though they still destroy bone structure. Many tumor cells produce parathyroid hormone related peptide, which increases bone resorption. This process of tumor-induced bone resorption leads to the release of growth factors stored in bone, which in turn increases tumor growth still further.

Bone destruction also occurs in the vast majority (over 80 percent) of patients with another type of cancer, multiple myeloma, which is a malignancy of the plasma cells that produce antibodies. The myeloma cells secrete cytokines, substances that may stimulate osteoclasts and inhibit osteoblasts. The bone destruction can cause severe bone pain, pathologic fractures, spinal cord compression, and life-threatening increases in blood calcium levels. A benign form of overproduction of antibodies, called monoclonal gammopathy, may also be associated with increased fracture risk.

Bone-resorbing cytokines are also produced in acute and chronic leukemia, Burkitt's lymphoma, and non-Hodgkins's lymphoma; patients with these chronic lymphopro-liferative disorders often have associated osteoporosis. Both osteoporosis and osteosclerosis (thickening of trabecular bone) have been reported in association with systemic mastocytosis, a condition of abnormal mast cell proliferation. In addition, there are other infiltrative processes that affect bone, including infections and marrow fibrosis (myelofibrosis).

Levels of the OSTEORISKMARKERS can be determined at the protein or nucleic acid level using any method known in the art. For example, at the nucleic acid level, Northern and Southern hybridization analysis, as well as ribonuclease protection assays using probes which specifically recognize one or more of these sequences can be used to determine gene expression. Alternatively, levels of OSTEORISKMARKERS can be measured using reverse-transcription-based PCR assays (RT-PCR), i.e., using primers specific for the differentially expressed sequence of genes. Levels of OSTEORISKMARKERS can also be determined at the protein level, i.e., by measuring the levels of peptides encoded by the gene products described herein, or activities thereof. Such methods are well known in the art and include, i.e., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the marker genes according to the activity of each protein analyzed.

The OSTEORISKMARKER proteins, polypeptides, mutations, and polymorphisms thereof can be detected in any suitable manner, but are typically detected by contacting a sample from the subject with an antibody which binds the OSTEORISKMARKER protein, polypeptide, mutation, or polymorphism and then detecting the presence or absence of a reaction product. The antibody may be monoclonal, polyclonal, chimeric, or a fragment of the foregoing, as discussed in detail above, and the step of detecting the reaction product may be carried out with any suitable immunoassay. The sample from the subject is typically a biological fluid as described above, and may be the same sample of biological fluid used to conduct the method described above.

Immunoassays carried out in accordance with the present invention may be homogeneous assays or heterogeneous assays. In a homogeneous assay the immunological reaction usually involves the specific antibody (i.e., anti-OSTEORISKMARKER protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunofluorescence methods, chemiluminescence methods, electrochemiluminescence or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.”

Antibodies can be conjugated to a solid support suitable for a diagnostic assay (i.e., beads such as protein A or protein G agarose, plates, slides or wells formed from materials such as latex or polystyrene) in accordance with known techniques, such as passive binding. Antibodies as described herein may likewise be conjugated to detectable labels or groups such as radiolabels (i.e., ³⁵S, ¹²⁵I, ¹³¹I), enzyme labels (i.e., horseradish peroxidase, alkaline phosphatase), and fluorescent labels (i.e., fluorescein, Alexa, green fluorescent protein) in accordance with known techniques.

Antibodies can also be useful for detecting post-translational modifications of OSTEORISKMARKER proteins, polypeptides, mutations, and polymorphisms, such as tyrosine phosphorylation, threonine phosphorylation, serine phosphorylation, glycosylation (i.e., O-GlcNAc). Such antibodies specifically detect the phosphorylated amino acids in a protein or proteins of interest, and can be used in immunoblotting, immunofluorescence, and ELISA assays described herein. These antibodies are well-known to those skilled in the art, and commercially available. Post-translational modifications can also be determined using metastable ions in reflector matrix-assisted laser desorption ionization-time of flight mass spectrometry (MALDI-TOF) (Wirth, U. et al. (2002) Proteomics 2(10): 1445-51).

For OSTEORISKMARKER proteins, polypeptides, mutations, and polymorphisms known to have enzymatic activity, the activities can be determined in vitro using enzyme assays known in the art. Such assays include, without limitation, kinase assays, phosphatase assays, reductase assays, among many others. Modulation of the kinetics of enzyme activities can be determined by measuring the rate constant KM using known algorithms, such as the Hill plot, Michaelis-Menten equation, linear regression plots such as Lineweaver-Burk analysis, and Scatchard plot.

Using sequence information provided by the database entries for the OSTEORISKMARKER sequences, expression of the OSTEORISKMARKER sequences can be detected (if present) and measured using techniques well known to one of ordinary skill in the art. For example, sequences within the sequence database entries corresponding to OSTEORISKMARKER sequences, or within the sequences disclosed herein, can be used to construct probes for detecting OSTEORISKMARKER RNA sequences in, i.e., Northern blot hybridization analyses or methods which specifically, and, preferably, quantitatively amplify specific nucleic acid sequences. As another example, the sequences can be used to construct primers for specifically amplifying the OSTEORISKMARKER sequences in, i.e., amplification-based detection methods such as reverse-transcription based polymerase chain reaction (RT-PCR). When alterations in gene expression are associated with gene amplification, deletion, polymorphisms, and mutations, sequence comparisons in test and reference populations can be made by comparing relative amounts of the examined DNA sequences in the test and reference cell populations.

Expression of the genes disclosed herein can be measured at the RNA level using any method known in the art. For example, Northern hybridization analysis using probes which specifically recognize one or more of these sequences can be used to determine gene expression. Alternatively, expression can be measured using reverse-transcription-based PCR assays (RT-PCR), i.e., using primers specific for the differentially expressed sequences. RNA can also be quantified using, for example, target amplification methods (TMA), bDNA methods such as signal amplification methods, and the like.

Alternatively, OSTEORISKMARKER protein and nucleic acid metabolites can be measured. The term “metabolite” includes any chemical or biochemical product of a metabolic process, such as any compound produced by the processing, cleavage or consumption of a biological molecule (i.e., a protein, nucleic acid, carbohydrate, or lipid). Metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, NMR and IR detection. (See, WO 04/056456 and WO 04/088309, each of which are hereby incorporated by reference in there entireties) In this regard, other OSTEORISKMARKER analytes can be measured using the above-mentioned detection methods, or other methods known to the skilled artisan. For example, circulating calcium ions (Ca²⁺) can be detected in a sample using fluorescent dyes such as the Fluo series, Fura-2A, Rhod-2, among others.

### Kits

The invention also includes an OSTEORISKMARKER-detection reagent, i.e., nucleic acids that specifically identify one or more OSTEORISKMARKER nucleic acids by having homologous nucleic acid sequences, such as oligonucleotide sequences, complementary to a portion of the OSTEORISKMARKER nucleic acids or antibodies to proteins encoded by the OSTEORISKMARKER nucleic acids packaged together in the form of a kit. The oligonucleotides can be fragments of the OSTEORISKMARKER genes. For example the oligonucleotides can be 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label. Instructions (i.e., written, tape, VCR, CD-ROM, etc.) for carrying out the assay may be included in the kit. The assay may for example be in the form of a Northern hybridization or a sandwich ELISA as known in the art.

For example, OSTEORISKMARKER detection reagents can be immobilized on a solid matrix such as a porous strip to form at least one OSTEORISKMARKER detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of OSTEORISKMARKERS present in the sample. The detection sites may be configured in any suitably detectable shape and are typically in the shape of a bar or dot spanning the width of a test strip.

Alternatively, the kit contains a nucleic acid substrate array comprising one or more nucleic acid sequences. The nucleic acids on the array specifically identify one or more nucleic acid sequences represented by OSTEORISKMARKERS 1-191. In various embodiments, the levels of 1, 2, 3, 4, 5, 6, 7, 8, 9, 10, 15, 20, 25, 40 or 50 or more of the sequences represented by OSTEORISKMARKERS 1-191 can be identified by virtue of binding to the array. The substrate array can be on, i.e., a solid substrate, i.e., a “chip” as described in U.S. Pat. No. 5,744,305. Alternatively, the substrate array can be a solution array, i.e., Luminex, Cyvera, Vitra and Quantum Dots' Mosaic.

Suitable sources for antibodies for the detection of OSTEORISKMARKERS include commercially available sources such as, for example, Abnova, EA, Biotrend, Accurate Chemical, Abcam, US Biologicals, Chemicon, DSHB, Assay Design, Inc., Sigma, Biogenesis, R&D, Linscott, Alpha Diagnostic International, Novus Biologicals, Serotec, Genetex, Genway Biotech, Biodesign, Aviva Systems Biology, Taconic Farms, Biovision, QED Bioscience Inc, BD Biosciences Pharmingen, Affinity Bioreagents, Bender, Calbiochem, Antigenix America, EMD Biosciences, Alpco Diagnostics, Anaspec, Imgenex, Phoenix Peptide, Invitrogen, American Diagnostics, Cell Sciences, Immundiagnostik, eBioscience, and Perkin Elmer. However, the skilled artisan can routinely make antibodies, nucleic acid probes, i.e., oligonucleotides, aptamers, siRNAs, antisense oligonucleotides, against any of the OSTEORISKMARKERS in Table 1.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Abdelrahaman, E., S. Raghavan, et al. (2005). “Racial difference in circulating sex hormone-binding globulin levels in prepubertal boys.” Metabolism 54(1): 91-6.
Abe, D., T. Sakaki, et al. (2005). “Metabolism of 2 alpha-propoxy-1 alpha,25-dihydroxyvitamin D3 and 2 alpha-(3-hydroxypropoxy)-1 alpha,25-dihydroxyvitamin D3 by human CYP27A1 and CYP24A1.” Drug Metab Dispos 33(6): 778-84.
Aggarwal, B. B., S. Shishodia, et al. (2006). “TNF blockade: an inflammatory issue.” Ernst Schering Res Found Workshop (56): 161-86.
Ahlstrom, M., M. Pekkinen, et al. (2005). “Dexamethasone down-regulates cAMP-phosphodiesterase in human osteosarcoma cells.” Biochem Pharmacol 69(2): 267-75.
Ahuja, S. S., S. Zhao, et al. (2003). “CD40 ligand blocks apoptosis induced by tumor necrosis factor alpha, glucocorticoids, and etoposide in osteoblasts and the osteocyte-like cell line murine long bone osteocyte-Y4.” Endocrinology 144(5): 1761-9.
Aitken, C. J., J. M. Hodge, et al. (2004). “Regulation of human osteoclast differentiation by thioredoxin binding protein-2 and redox-sensitive signaling.” J Bone Miner Res 19(12): 2057-64.
Akesson, K., S. M. Kakonen, et al. (2005). “Fracture-induced changes in bone turnover: a potential confounder in the use of biochemical markers in osteoporosis.” J Bone Miner Metab 23(1): 30-5.
Akune, T., S. Ohba, et al. (2004). “PPARgamma insufficiency enhances osteogenesis through osteoblast formation from bone marrow progenitors.” J Clin Invest 113(6): 846-55.
Alatalo, S. L., Z. Peng, et al. (2003). “A novel immunoassay for the determination of tartrate-resistant acid phosphatase 5b from rat serum.” J Bone Miner Res 18(1): 134-9.
Albagha, 0. M., P. N. Tasker, et al. (2002). “Linkage disequilibrium between polymorphisms in the human TNFRSF1B gene and their association with bone mass in perimenopausal women.” Hum Mol Genet. 11(19): 2289-95.
Arey, B. J., R. Seethala, et al. (2005). “A novel calcium-sensing receptor antagonist transiently stimulates parathyroid hormone secretion in vivo.” Endocrinology 146(4): 2015-22.
Armagan, O., T. Uz, et al. (2005). “Serological screening for celiac disease in premenopausal women with idiopathic osteoporosis.” Clin Rheumatol 24(3): 239-43.
Aronson, J. (2004). “Modulation of distraction osteogenesis in the aged rat by fibroblast growth factor.” Clin Orthop Relat Res (425): 264-83.
Bajnok, E., I. Takacs, et al. (2000). “Lack of association between interleukin-1 receptor antagonist protein gene polymorphism and bone mineral density in Hungarian postmenopausal women.” Bone 27(4): 559-62.
Baldock, P. A. and J. A. Eisman (2004). “Genetic determinants of bone mass.” Curr Opin Rheumatol 16(4): 450-6.
Balemans, W. and W. Van Hul (2004). “Identification of the disease-causing gene in sclerosteosis—discovery of a novel bone anabolic target?” J Musculoskelet Neuronal Interact 4(2): 139-42.
Baribault, H., J. Danao, et al. (2006). “The G-Protein-Coupled Receptor GPR103 Regulates Bone Formation.” Mol Cell Biol 26(2): 709-17.
Ben Lagha, N., P. Menuelle, et al. (2002). “Bone formation in the context of growth retardation induced by hIGFBP-1 overexpression in transgenic mice.” Connect Tissue Res 43(2-3): 515-9.
Bodine, P. V. and B. S. Komm (2006). “Wnt signaling and osteoblastogenesis.” Rev Endocr Metab Disord 7(1-2): 33-39.
Bronsky, J. and R. Prusa (2004). “Amylin fasting plasma levels are decreased in patients with osteoporosis.” Osteoporos Int 15(3): 243-7.
Cason, A. L., Y. Ikeguchi, et al. (2003). “X-linked spermine synthase gene (SMS) defect: the first polyamine deficiency syndrome.” Eur J Hum Genet. 11(12): 937-44.
Cetani, F., E. Pardi, et al. (2003). “Calcium-sensing receptor gene polymorphism is not associated with bone mineral density in Italian postmenopausal women.” Eur J Endocrinol 148(6): 603-7.
Chalhoub, N., N. Benachenhou, et al. (2003). “Grey-lethal mutation induces severe malignant autosomal recessive osteopetrosis in mouse and human.” Nat Med 9(4): 399-406.
Chen, F. P., T. Hsu, et al. (2004). “Expression of estrogen receptors alpha and beta in human osteoblasts: identification of exon-2 deletion variant of estrogen receptor beta in postmenopausal women.” Chang Gung Med J 27(2): 107-15.
Chen, X. D., X. Bian, et al. (2005). “Dissection of the sets of genes that control the behavior of biglycan-deficient pre-osteoblasts using oligonucleotide microarrays.” Bone 37(2): 192-203.
Deckelbaum, R. A., A. Majithia, et al. (2006). “The homeoprotein engrailed 1 has pleiotropic functions in calvarial intramembranous bone formation and remodeling.” Development 133(1): 63-74.
Do, S. H., W. I. Jeong, et al. (2006). “Alcohol-induced bone degradation and its early detection in the alcohol-fed castrated rats.” Mol Cell Biochem 282(1-2): 45-52.
Dresner-Pollak, R., N. Gelb, et al. (2004). “Interleukin 10-deficient mice develop osteopenia, decreased bone formation, and mechanical fragility of long bones.” Gastroenterology 127(3): 792-801.
Eastell, R., J. H. Krege, et al. (2006). “Development of an algorithm for using PINP to monitor treatment of patients with teriparatide.” Curr Med Res Opin 22(1): 61-6.
Effros, R. B. (2004). “Replicative senescence of CD8 T cells: effect on human ageing.” Exp Gerontol 39(4): 517-24.
Enattah, N., V. V. Valimaki, et al. (2004). “Molecularly defined lactose malabsorption, peak bone mass and bone turnover rate in young finnish men.” Calcif Tissue Int 75(6): 488-93.
Enjuanes, A., N. Garcia-Giralt, et al. (2005). “A new SNP in a negative regulatory region of the CYP19A1 gene is associated with lumbar spine BMD in postmenopausal women.” Bone.
Erbagci, A. B., M. Araz, et al. (2002). “Serum prolidase activity as a marker of osteoporosis in type 2 diabetes mellitus.” Clin Biochem 35(4): 263-8.
Ershler, W. B. and E. T. Keller (2000). “Age-associated increased interleukin-6 gene expression, late-life diseases, and frailty.” Annu Rev Med 51: 245-70.
Ersoy, F. F., S. P. Passadakis, et al. (2006). “Bone mineral density and its correlation with clinical and laboratory factors in chronic peritoneal dialysis patients.” J Bone Miner Metab 24(1): 79-86.
Ezura, Y., M. Kajita, et al. (2004). “Association of multiple nucleotide variations in the pituitary glutaminyl cyclase gene (QPCT) with low radial BMD in adult women.” J Bone Miner Res 19(8): 1296-301.
Ezura, Y., T. Nakajima, et al. (2003). “Association of molecular variants, haplotypes, and linkage disequilibrium within the human vitamin D-binding protein (DBP) gene with postmenopausal bone mineral density.” J Bone Miner Res 18(9): 1642-9.
Fan, Y., J. Liu, et al. (2005). “Functional proteome of bones in rats with osteoporosis following ovariectomy.” Life Sci 76(25): 2893-901.
Flanagan, A. M., H. M. Massey, et al. (2002). “Macrophage colony-stimulating factor and receptor activator NF-kappaB ligand fail to rescue osteoclast-poor human malignant infantile osteopetrosis in vitro.” Bone 30(1): 85-90.
Georgescu, C., T. Seck, et al. (2004). “Bone matrix insulin-like growth factor (IGF)-I, IGF-II and transforming growth factor (TGF)-beta1 levels in men and postmenopausal women with osteoporosis: lack of association with circulating growth factors and bone mineral density.” Rev Med Chir Soc Med Nat Iasi 108(2): 281-6.
Golbahar, J., M. A. Aminzadeh, et al. (2005). “Association of red blood cell 5-methyltetrahydrofolate folate with bone mineral density in postmenopausal Iranian women.” Osteoporos Int 16(12): 1894-8.
Grewal, T. S., P. G. Genever, et al. (2000). “Best5: a novel interferon-inducible gene expressed during bone formation.” Faseb J 14(3): 523-31.
Gur, A., A. Denli, et al. (2003). “The effects of alendronate and calcitonin on cytokines in postmenopausal osteoporosis: a 6-month randomized and controlled study.” Yonsei Med J 44(1): 99-109.
Hayer, S., G. Steiner, et al. (2005). “CD44 is a determinant of inflammatory bone loss.” J Exp Med 201(6): 903-14.
Heiss, C., L. M. Hoesel, et al. (2004). “Vitamin K in combination with other biochemical markers to diagnose osteoporosis.” Biomarkers 9(6): 479-88.
Herrmann, M., T. Widmann, et al. (2005). “Increased osteoclast activity in the presence of increased homocysteine concentrations.” Clin Chem 51(12): 2348-53.
Idris, A. I., R. J. van 't H of, et al. (2005). “Regulation of bone mass, bone loss and osteoclast activity by cannabinoid receptors.” Nat Med 11(7): 774-9.
Ishida, R., M. Emi, et al. (2003). “Association of a haplotype (196Phe/532Ser) in the interleukin-1-receptor-associated kinase (IRAK1) gene with low radial bone mineral density in two independent populations.” J Bone Miner Res 18(3): 419-23.
Ishida, R., Y. Ezura, et al. (2001). “Linkage disequilibrium and haplotype analysis among four novel single-nucleotide polymorphisms in the human leukemia inhibitory factor (LIF) gene.” J Hum Genet. 46(10): 557-9.
Jadlowiec, J., D. Dongell, et al. (2005). “Pregnancy-associated plasma protein-a is involved in matrix mineralization of human adult mesenchymal stem cells and angiogenesis in the chick chorioallontoic membrane.” Endocrinology 146(9): 3765-72.
Jiao, J., X. W. Meng, et al. (2004). “[Bone mineral density and leptin receptor polymorphism Gln223Arg in Han women in Beijing].” Zhonghua Nei Ke Za Zhi 43(4): 276-9.
Johnson-Pais, T. L., F. R. Singer, et al. (2003). “Identification of a novel tandem duplication in exon 1 of the TNFRSF11A gene in two unrelated patients with familial expansile osteolysis.” J Bone Miner Res 18(2): 376-80.
Kahl, K. G., S. Rudolf, et al. (2005). “Bone mineral density, markers of bone turnover, and cytokines in young women with borderline personality disorder with and without comorbid major depressive disorder.” Am J Psychiatry 162(1): 168-74.
Kajita, M., Y. Ezura, et al. (2003). “Association of the −381T/C promoter variation of the brain natriuretic peptide gene with low bone-mineral density and rapid postmenopausal bone loss.” J Hum Genet. 48(2): 77-81.
Kamel Mohamed, S. G., E. Sugiyama, et al. (2005). “Interleukin-4 inhibits RANKL-induced expression of NFATcl and c-Fos: a possible mechanism for downregulation of osteoclastogenesis.” Biochem Biophys Res Commun 329(3): 839-45.
Keller, B., A. Volkmann, et al. (2006). “Bioinformatic identification and characterization of new members of short-chain dehydrogenase/reductase superfamily.” Mol Cell Endocrinol 248(1-2): 56-60.
Kim, G. S., J. M. Koh, et al. (2005). “Association of the OSCAR promoter polymorphism with BMD in postmenopausal women.” J Bone Miner Res 20(8): 1342-8.
Kim, S. H., S. Jun, et al. (2005). “Identification of parathyroid hormone-regulated proteins in mouse bone marrow cells by proteomics.” Biochem Biophys Res Commun 330(2): 423-9.
Koga, T., Y. Matsui, et al. (2005). “NFAT and Osterix cooperatively regulate bone formation.” Nat Med 11(8): 880-5.
Koh, J. M., Y. H. Khang, et al. (2005). “Higher circulating hsCRP levels are associated with lower bone mineral density in healthy pre- and postmenopausal women: evidence for a link between systemic inflammation and osteoporosis.” Osteoporos Int 16(10): 1263-71.
Kokubu, C., U. Heinzmann, et al. (2004). “Skeletal defects in ringelschwanz mutant mice reveal that Lrp6 is required for proper somitogenesis and osteogenesis.” Development 131(21): 5469-80.
Kronenberg, H. M. (2002). “NPT2a—the key to phosphate homeostasis.” N Engl J Med 347(13): 1022-4.
Lee, G. S., K. C. Choi, et al. (2003). “Expression of human Calbindin-D(9k) correlated with age, vitamin D receptor and blood calcium level in the gastrointestinal tissues.” Clin Biochem 36(4): 255-61.
Lemos, M. C., P. Kotanko, et al. (2005). “A novel EXT1 splice site mutation in a kindred with hereditary multiple exostosis and osteoporosis.” J Clin Endocrinol Metab 90(9): 5386-92.
Letizia, C., A. Taranta, et al. (2004). “Type II benign osteopetrosis (Albers-Schonberg disease) caused by a novel mutation in CLCN7 presenting with unusual clinical manifestations.” Calcif Tissue Int 74(1): 42-6.
Liu, X. H., Y. J. Liu, et al. (2003). “No evidence for linkage and/or association of human alpha2-HS glycoprotein gene with bone mineral density variation in Chinese nuclear families.” Calcif Tissue Int 73(3): 244-50.
Liu, Y. Z., V. Dvomyk, et al. (2005). “A novel pathophysiological mechanism for osteoporosis suggested by an in vivo gene expression study of circulating monocytes.” J Biol Chem 280(32): 29011-6.
Liu, Y. Z., Y. J. Liu, et al. (2003). “Molecular studies of identification of genes for osteoporosis: the 2002 update.” J Endocrinol 177(2): 147-96.
Loughlin, J., B. Dowling, et al. (2004). “Functional variants within the secreted frizzled-related protein 3 gene are associated with hip osteoarthritis in females.” Proc Natl Acad Sci USA 101(26): 9757-62.
Martignetti, J. N.Y., NY, United States of America, 0. B. Dowling, NY, United States of America (iNVENTORS), et al. (2005). “US20050221336A1: Mutations in capillary morphogenesis gene-2 (CMG-2) and use thereof.
Matsuo, K., J. M. Owens, et al. (2000). “Fosl1 is a transcriptional target of c-Fos during osteoclast differentiation. “Nat Genet. 24(2): 184-7.
Moreno, M. L., J. B. Crusius, et al. (2005). “The IL-1 gene family and bone involvement in celiac disease.” Immunogenetics 57(8): 618-20.
Morris, H. A. (2005). “Vitamin D: A Hormone for All Seasons—How much is enough? Understanding the New Pressures.” Clin Biochem Rev 26(1): 21-32.
Munz, W., M. E. Hammadeh, et al. (2004). “Serum inhibin A, inhibin B, pro-alphaC, and activin A levels in women with idiopathic premature ovarian failure.” Fertil Steril 82(3): 760-2.
Muratli, H. H., L. Celebi, et al. (2005). “Comparison of plasma endothelin levels between osteoporotic, osteopenic and normal subjects.” BMC Musculoskelet Disord 6: 49.
Napoli, N., D. T. Villareal, et al. (2005). “Effect of CYP1A1 gene polymorphisms on estrogen metabolism and bone density.” J Bone Miner Res 20(2): 232-9.
Needleman, P. and P. T. Manning (1999). “Interactions between the inducible cyclooxygenase (COX-2) and nitric oxide synthase (iNOS) pathways: implications for therapeutic intervention in osteoarthritis.” Osteoarthritis Cartilage 7(4): 367-70.
Negri, A. L. (2005). “The klotho gene: a gene predominantly expressed in the kidney is a fundamental regulator of aging and calcium/phosphorus metabolism.” J Nephrol 18(6): 654-8.
Nguyen, T. V., L. M. Esteban, et al. (2005). “Contribution of the collagen 1 alpha1 and vitamin D receptor genes to the risk of hip fracture in elderly women.” J Clin Endocrinol Metab 90(12): 6575-9.
Norrdin, R. W., W. S. Jee, et al. (1990). “The role of prostaglandins in bone in vivo.” Prostaglandins Leukot Essent Fatty Acids 41(3): 139-49.
Ofek, O., M. Karsak, et al. (2006). “Peripheral cannabinoid receptor, CB2, regulates bone mass.” Proc Natl Acad Sci USA.
Ofluoglu, D., E. Karadag-Saygi, et al. (2006). “Early effect of nasal salmon calcitonin on the bone marker Crosslaps.” Rheumatol Int 26(4): 288-91.
Ogata, N., M. Shiraki, et al. (2001). “A polymorphic variant at the Werner helicase (WRN) gene is associated with bone density, but not spondylosis, in postmenopausal women.” J Bone Miner Metab 19(5): 296-301.
Olivier, E., E. Soury, et al. (2000). “Fetuin-B, a second member of the fetuin family in mammals.” Biochem J 350 Pt 2: 589-97.
Omasu, F., Y. Ezura, et al. (2003). “Association of genetic variation of the RIL gene, encoding a PDZ-LIM domain protein and localized in 5q31.1, with low bone mineral density in adult Japanese women.” J Hum Genet. 48(7): 342-5.
Ozeren, M., S. C. Karahan, et al. (2005). “The effects of short-term raloxifene therapy on fibrinolysis markers: TAFI, tPA, and PAI-1.” Acta Obstet Gynecol Scand 84(10): 987-91.
Paloneva, J., J. Mandelin, et al. (2003). “DAP12/TREM2 deficiency results in impaired osteoclast differentiation and osteoporotic features.” J Exp Med 198(4): 669-75.
Papadopoulou, F., G. E. Krassas, et al. (2004). “Serum leptin values in relation to bone density and growth hormone-insulin like growth factors axis in healthy men.” Arch Androl 50(2): 97-103.
Peng, W. J., J. L. Jiang, et al. (2005). “Asymmetric dimethylarginine is not involved in ovariectomy-induced osteopenia in rats.” Comp Med 55(1): 30-3.
Pescarmona, G. P., P. D'Amelio, et al. (2001). “Haptoglobin genotype as a risk factor for postmenopausal osteoporosis.” J Med Genet. 38(9): 636-8.
Petersson, M., A. Lagumdzija, et al. (2002). “Oxytocin stimulates proliferation of human osteoblast-like cells.” Peptides 23(6): 1121-6.
Philip, B., Z. Lu, et al. (2005). “Regulation of GDF-8 signaling by the p38 MAPK.” Cell Signal 17(3): 365-75.
Pouilles, J. M., F. A. Tremollieres, et al. (2006). “Osteoporosis in otherwise healthy perimenopausal and early postmenopausal women: Physical and biochemical characteristics.” Osteoporos Int 17(2): 193-200.
Pufe, T., K. E. Scholz-Ahrens, et al. (2003). “The role of vascular endothelial growth factor in glucocorticoid-induced bone loss: evaluation in a minipig model.” Bone 33(6): 869-76.
Qin, L., J. Tamasi, et al. (2005). “Amphiregulin is a novel growth factor involved in normal bone development and in the cellular response to parathyroid hormone stimulation.” J Biol Chem 280(5): 3974-81.
Reneland, R. H., S. Mah, et al. (2005). “Association between a variation in the phosphodiesterase 4D gene and bone mineral density.” BMC Med Genet. 6: 9.
Rusinska, A. and D. Chlebna-Sokol (2006). “Insulin-like growth factor-I and mineral metabolism markers in children with idiopathic decrease in bone mass.” Clin Chim Acta 366(1-2): 257-63.
Sahin, G., H. Guler, et al. (2005). “The effect of circulating nitric oxide level on axial bone mineral density in postmenopausal Turkish women with rheumatoid arthritis: a preliminary report.” Rheumatol Int: 1-5.
Salamone, L. M., J. A. Cauley, et al. (2000). “Apolipoprotein E gene polymorphism and bone loss: estrogen status modifies the influence of apolipoprotein E on bone loss.” J Bone Miner Res 15(2): 308-14.
Seibel, M. J. (2005). “Biochemical markers of bone turnover: part I: biochemistry and variability.” Clin Biochem Rev 26(4): 97-122.
Shimizu, M., K. Higuchi, et al. (2002). “A congenic mouse and candidate gene at the Chromosome 13 locus regulating bone density.” Mamm Genome 13(7): 335-40.
Sudo, Y., Y. Ezura, et al. (2005). “Association of single nucleotide polymorphisms in the promoter region of the pro-opiomelanocortin gene (POMC) with low bone mineral density in adult women.” J Hum Genet. 50(5): 235-40.
Tabb, M. M., A. Sun, et al. (2003). “Vitamin K2 regulation of bone homeostasis is mediated by the steroid and xenobiotic receptor SXR.” J Biol Chem 278(45): 43919-27.
Takeda, E., H. Yamamoto, et al. (2004). “Inorganic phosphate homeostasis and the role of dietary phosphorus.” J Cell Mol Med 8(2): 191-200.
Takeshita, S., R. Kikuno, et al. (1993). “Osteoblast-specific factor 2: cloning of a putative bone adhesion protein with homology with the insect protein fasciclin I.” Biochem J 294 (Pt 1): 271-8.
Tian, E., F. Zhan, et al. (2003). “The role of the Wnt-signaling antagonist DKK1 in the development of osteolytic lesions in multiple myeloma.” N Engl J Med 349(26): 2483-94.
Torday, J. S. (2003). “Parathyroid hormone-related protein is a gravisensor in lung and bone cell biology.” Adv Space Res 32(8): 1569-76.
Tsai, F. J., W. C. Chen, et al. (2003). “The ALUI calcitonin receptor gene polymorphism (TT) is associated with low bone mineral density and susceptibility to osteoporosis in postmenopausal women.” Gynecol Obstet Invest 55(2): 82-7.
Uusitalo, H., J. Rantakokko, et al. (2005). “Bone defect repair in immobilization-induced osteopenia: a pQCT, biomechanical, and molecular biologic study in the mouse femur.” Bone 36(1): 142-9.
Van Hul, E., J. Gram, et al. (2002). “Localization of the gene causing autosomal dominant osteopetrosis type I to chromosome 11q12-13.” J Bone Miner Res 17(6): 1111-7.
Vaughan, T., J. A. Pasco, et al. (2002). “Alleles of RUNX2/CBFA1 gene are associated with differences in bone mineral density and risk of fracture.” J Bone Miner Res 17(8): 1527-34.
Wu, H. and J. R. Arron (2003). “TRAF6, a molecular bridge spanning adaptive immunity, innate immunity and osteoimmunology.” Bioessays 25(11): 1096-105.
Yamada, Y., F. Ando, et al. (2005). “Association of polymorphisms in CYP17A1, MTP, and VLDLR with bone mineral density in community-dwelling Japanese women and men.” Genomics 86(1): 76-85.
Yamagishi, S., K. Nakamura, et al. (2005). “Possible participation of advanced glycation end products in the pathogenesis of osteoporosis in diabetic patients.” Med Hypotheses 65(6): 1013-5.
Yu, X., S. Chen, et al. (2005). “Neurofibromin and its inactivation of Ras are prerequisites for osteoblast functioning.” Bone 36(5): 793-802.
Zhao, H., H. Kitaura, et al. (2005). “Critical Role of beta3 Integrin in Experimental Postmenopausal Osteoporosis.” J Bone Miner Res 20(12): 2116-23.
Zhong, N., R. A. Garman, et al. (2005). “Gene expression patterns in bone after 4 days of hind-limb unloading in two inbred strains of mice.” Aviat Space Environ Med 76(6): 530-5.
Ziegler, S., S. Kudlacek, et al. (2005). “Osteoprotegerin plasma concentrations correlate with severity of peripheral artery disease.” Atherosclerosis 182(1): 175-80.
Zmuda, J. M., Y. T. Sheu, et al. (2005). “Genetic epidemiology of osteoporosis: past, present, and future.” Curr Osteoporos Rep 3(3): 111-5.
Zofkova, I., R. Bahbouh, et al. (2000). “The pathophysiological implications of circulating androgens on bone mineral density in a normal female population.” Steroids 65(12): 857-61.

1. A method with a predetermined level of predictability for assessing a risk of development of osteoporosis, pre-osteoporosis, or bone fracture in a subject comprising:
a. measuring the level of an effective amount of one or more OSTEORISKMARKERS selected from the group consisting of OSTEORISKMARKERS 1-191 in a sample from the subject, and
b. measuring a clinically significant alteration in the level of the one or more OSTEORISKMARKERS in the sample, wherein the alteration indicates an increased risk of developing osteoporosis, pre-osteoporosis, or bone fracture in the subject.

2.-13. (canceled)

14. A method with a predetermined level of predictability for diagnosing or identifying a subject having osteoporosis or pre-osteoporosis comprising:
a. measuring the level of an effective amount of one or more OSTEORISKMARKERS selected from the group consisting of OSTEORISKMARKERS 1-191 in a sample from the subject, and
b. comparing the level of the effective amount of the one or more OSTEORISKMARKERS to a reference value.

15.-17. (canceled)

18. A method with a predetermined level of predictability for assessing the progression of osteoporosis or pre-osteoporosis in a subject, comprising:
a. detecting the level of an effective amount of one or more OSTEORISKMARKERS selected from the group consisting of OSTEORISKMARKERS 1-191 in a first sample from the subject at a first period of time;
b. optionally detecting the level of an effective amount of one or more OSTEORISKMARKERS in a second sample from the subject at a second period of time;
c. comparing the level of the effective amount of the one or more OSTEORISKMARKERS detected in step (a) to the amount detected in step (b), or to a reference value.

19.-25. (canceled)

26. A method with a predetermined level of predictability for assessing the progression of diminished bone mass associated with osteoporosis or pre-osteoporosis in a subject comprising:
a. detecting the level of an effective amount of one or more OSTEORISKMARKERS selected from the group consisting of OSTEORISKMARKERS 1-191 in a first sample from the subject at a first period of time;
b. optionally detecting the level of an effective amount of one or more OSTEORISKMARKERS in a second sample from the subject at a second period of time;
c. comparing the level of the effective amount of the one or more OSTEORISKMARKERS detected in step (a) to the amount detected in step (b), or to a reference value.

27.-33. (canceled)

34. A method with a predetermined level of predictability for monitoring the effectiveness of treatment for osteoporosis or pre-osteoporosis in a subject comprising:
a. detecting the level of an effective amount of one or more OSTEORISKMARKERS selected from the group consisting of OSTEORISKMARKERS 1-191 in a first sample from the subject at a first period of time;
b. optionally detecting the level of an effective amount of one or more OSTEORISKMARKERS in a second sample from the subject at a second period of time;
c. comparing the level of the effective amount of the one or more OSTEORISKMARKERS detected in step (a) to the amount detected in step (b), or to a reference value, wherein the effectiveness of treatment is monitored by a change in the level of the effective amount of one or more OSTEORISKMARKERS from the subject.

35.-42. (canceled)

43. A method with a predetermined level of predictability for selecting a treatment regimen for a subject diagnosed with or at risk for osteoporosis or pre-osteoporosis comprising:
a. detecting the level of an effective amount of one or more OSTEORISKMARKERS selected from the group consisting of OSTEORISKMARKERS 1-191 in a first sample from the subject at a first period of time;
b. optionally detecting the level of an effective amount of one or more OSTEORISKMARKERS in a second sample from the subject at a second period of time;
c. comparing the level of the effective amount of the one or more OSTEORISKMARKERS detected in step (a) to a reference value, or optionally, to the amount detected in step (b).

44.-52. (canceled)

53. An osteoporosis or pre-osteoporosis reference molecular profile, comprising a pattern of marker levels of an effective amount of one or more markers selected from the group consisting of OSTEORISKMARKERS 1-191, taken from one or more subjects who do not have osteoporosis or pre-osteoporosis.

54. An osteoporosis or pre-osteoporosis subject molecular profile, comprising a pattern of marker levels of an effective amount of one or more markers selected from the group consisting of OSTEORISKMARKERS 1-191 taken from one or more subjects who have osteoporosis or pre-osteoporosis, are at risk for developing osteoporosis or pre-osteoporosis, or are being treated for osteoporosis or pre-osteoporosis.

55.-59. (canceled)

60. An OSTEORISKMARKER panel comprising one or more OSTEORISKMARKERS that are indicative of one or more physiological functions or canonical molecular pathways associated with osteoporosis or pre-osteoporosis.

61.-69. (canceled)

70. An OSTEORISKMARKER panel comprising one or more OSTEORISKMARKERS selected from at least one cluster of OSTEORISKMARKERS defined by the relative proximity of each OSTEORISKMARKER to other cluster member OSTEORISKMARKERS in and across canonical molecular pathways or by the relative correlation of each OSTEORISKMARKER with other cluster member OSTEORISKMARKERS.

71.-73. (canceled)

74. A method for treating one or more subjects at risk for developing osteoporosis or pre-osteoporosis, comprising:
a. detecting the presence of increased levels of one or more OSTEORISKMARKERS present in a sample from the one or more subjects; and
b. treating the one or more subjects with one or more bone mineral content-modulating drugs until altered levels of the one or more OSTEORISKMARKERS return to a baseline value measured in one or more subjects at low risk for developing osteoporosis or pre-osteoporosis, or a baseline value measured in one or more subjects who show improvements in osteoporosis or pre-osteoporosis risk markers as a result of treatment with one or more bone mineral content-modulating drugs.

75.-79. (canceled)

80. A method of evaluating changes in the risk of bone fracture or diminished bone mass in a subject diagnosed with or at risk for developing pre-osteoporosis, comprising:
a. detecting the level of an effective amount of one or more OSTEORISKMARKERS selected from the group consisting of OSTEORISKMARKERS 1-191 in a first sample from the subject at a first period of time;
b. optionally detecting the level of an effective amount of one or more OSTEORISKMARKERS in a second sample from the subject at a second period of time;
c. comparing the level of the effective amount of the one or more OSTEORISKMARKERS detected in step (a) to a reference value, or optionally, the amount in step (b).

81.-93. (canceled)

94. In a method of diagnosing or identifying a subject at risk for developing osteoporosis or pre-osteoporosis by analyzing osteoporosis or pre-osteoporosis risk factors, the improvement comprising:
a. measuring the level of an effective amount of one or more OSTEORISKMARKERS selected from the group consisting of OSTEORISKMARKERS 1-191 in a sample from the subject, and
b. measuring a clinically significant alteration in the level of the one or more OSTEORISKMARKERS in the sample, wherein the alteration indicates an increased risk of developing osteoporosis or pre-osteoporosis in the subject.

95.-96. (canceled)
